SEQUENCE LISTING (72 sequences)

SEQ ID NO: 1 - protein, 5 aa, Mus musculus
TYAMN

SEQ ID NO: 2 - protein, 19 aa, Mus musculus
RIRSKYNNYATYYADSVKG

SEQ ID NO: 3 - protein, 14 aa, Mus musculus
HGNFGNSYVSWFAY

SEQ ID NO: 4 - protein, 14 aa, Mus musculus
GSSTGAVTTSNYAN

SEQ ID NO: 5 - protein, 7 aa, Mus musculus
GTNKRAP

SEQ ID NO: 6 - protein, 9 aa, Mus musculus
ALWYSNLWV

SEQ ID NO: 7 - protein, 125 aa, Mus musculus
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 8 - protein, 109 aa, Mus musculus
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 116 aa, Artificial Sequence
  83A10 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 10 - protein, 116 aa, Artificial Sequence
  Mab21 VH, Mab22 VH, Mab42 VH
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSS

SEQ ID NO: 11 - protein, 109 aa, Artificial Sequence
  83A10 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 12 - protein, 109 aa, Artificial Sequence
  Mab21 VL, Mab27 VL, Mab33 VL, Mab39 VL
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIK

SEQ ID NO: 13 - protein, 109 aa, Artificial Sequence
  Mab22 VL
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIK

SEQ ID NO: 14 - protein, 109 aa, Artificial Sequence
  Mab42 VL
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIK

SEQ ID NO: 15 - protein, 5 aa, Artificial Sequence
  83A10 CDR1H
SYAMS

SEQ ID NO: 16 - protein, 17 aa, Artificial Sequence
  83A10 CDR2H
AISGSGGSTYYADSVKG

SEQ ID NO: 17 - protein, 7 aa, Artificial Sequence
  83A10 CDR3H, Mab21 CDR3H, Mab22 CDR3H, Mab42 CDR3H, Mab 27 CDR3H, Mab33 CDR3H, Mab39 CDR3H
VLGWFDY

SEQ ID NO: 18 - protein, 12 aa, Artificial Sequence
  83A10 CDR1L
RASQSVSSSYLA

SEQ ID NO: 19 - protein, 7 aa, Artificial Sequence
  83A10 CDR2L
GASSRAT

SEQ ID NO: 20 - protein, 10 aa, Artificial Sequence
  83A10 CDR3L, Mab21 CDR3L, Mab22 CDR3L, Mab42 CDR3L
QQYGYPPDFT

SEQ ID NO: 21 - protein, 5 aa, Artificial Sequence
  Mab21 CDR1H, Mab22 CDR1H, Mab42 CDR1H
DNAMG

SEQ ID NO: 22 - protein, 17 aa, Artificial Sequence
  Mab21 CDR2H, Mab22 CDR2H, Mab42 CDR2H
AISGPGSSTYYADSVKG

SEQ ID NO: 23 - protein, 13 aa, Artificial Sequence
  Mab21 CDR1L
RASQSVSEYYLAW

SEQ ID NO: 24 - protein, 8 aa, Artificial Sequence
  Mab21 CDR2L
EHASTRAT

SEQ ID NO: 25 - protein, 12 aa, Artificial Sequence
  Mab22 CDR1L
RASQSVSSYYLA

SEQ ID NO: 26 - protein, 7 aa, Artificial Sequence
  Mab22 CDR2L
GAGSRAT

SEQ ID NO: 27 - protein, 12 aa, Artificial Sequence
  Mab42 CDR1L
RASQSVSDEYLS

SEQ ID NO: 28 - protein, 7 aa, Artificial Sequence
  Mab42 CDR2L
SASTRAT

SEQ ID NO: 29 - protein, 5 aa, Artificial Sequence
  Mab27 CDR1H
SAPMG

SEQ ID NO: 30 - protein, 16 aa, Artificial Sequence
  Mab27 CDR2H
AISYIGHTYYADSVKG

SEQ ID NO: 31 - protein, 12 aa, Artificial Sequence
  Mab27 CDR1L, Mab33 CDR1L, Mab39 CDR1L
RASQSVSEYYLA

SEQ ID NO: 32 - protein, 7 aa, Artificial Sequence
  Mab27 CDR2L, Mab33 CDR2L, Mab39 CDR2L
HASTRAT

SEQ ID NO: 33 - protein, 10 aa, Artificial Sequence
  Mab27 CDR3L, Mab33 CDR3L, Mab39 CDR3L
QQYGYPPDFT

SEQ ID NO: 34 - protein, 5 aa, Artificial Sequence
  Mab33 CDR1H
TNAMG

SEQ ID NO: 35 - protein, 17 aa, Artificial Sequence
  Mab33 CDR2H
AINRFGGSTYYADSVKG

SEQ ID NO: 36 - protein, 5 aa, Artificial Sequence
  Mab39 CDR1H
QNAMG

SEQ ID NO: 37 - protein, 17 aa, Artificial Sequence
  Mab39 CDR2H
AISPTGFSTYYADSVKG

SEQ ID NO: 38 - protein, 115 aa, Artificial Sequence
  Mab27 VH
EVQLLESGGGLVQPGGSLRLSCAASGFTFSSAPMGWVRQAPGKGLEWVSAISYIGHTYYADSVKGRFTISRDNSKNTLYLQMNSLRAEDTAVYYCAKVLGWFDYWGQGTLVTVSS

SEQ ID NO: 39 - protein, 116 aa, Artificial Sequence
  Mab33 VH
EVQLLESGGGLVQPGGSLRLSCAASGFTFYTNAMGWVRQAPGKGLEWVSAINRFGGSTYYADSVKGRFTISRDNSKNTLYLQMNSLRAEDTAVYYCAKVLGWFDYWGQGTLVTVSS

SEQ ID NO: 40 - protein, 116 aa, Artificial Sequence
  Mab39 VH
EVQLLESGGGLVQPGGSLRLSCAASGFTFTQNAMGWVRQAPGKGLEWVSAISPTGFSTYYADSVKGRFTISRDNSKNTLYLQMNSLRAEDTAVYYCAKVLGWFDYWGQGTLVTVSS

SEQ ID NO: 41 - protein, 103 aa, Artificial Sequence
  83A10 BCMA CH1, Mab21 BCMA CH1, Mab22 BCMA CH1, Mab42 BCMA CH1
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 42 - protein, 107 aa, Artificial Sequence
  83A10 BCMA CL, Mab21 BCMA CL, Mab22 BCMA CL, Mab42 BCMA CL
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 103 aa, Artificial Sequence
  CD3 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 107 aa, Artificial Sequence
  CD3 CL
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 671 aa, Artificial Sequence
  83A10-TCBcv knob HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 446 aa, Artificial Sequence
  83A10-TCBcv hole HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 216 aa, Artificial Sequence
  83A10-TCBcv LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 232 aa, Artificial Sequence
  CD3 LC
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 671 aa, Artificial Sequence
  21-TCBcv knob HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 446 aa, Artificial Sequence
  21-TCBcv hole HC
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 216 aa, Artificial Sequence
  21-TCBcv LC
EIVLTQSPGTLSLSPGERATLSCRASQSVSEYYLAWYQQKPGQAPRLLIEHASTRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 671 aa, Artificial Sequence; identical to SEQ ID NO: 49
  22-TCBcv knob HC

SEQ ID NO: 53 - protein, 446 aa, Artificial Sequence; identical to SEQ ID NO: 50
  22-TCBcv hole HC

SEQ ID NO: 54 - protein, 216 aa, Artificial Sequence
  22-TCBcv LC
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 671 aa, Artificial Sequence; identical to SEQ ID NO: 49
  42-TCBcv knob HC

SEQ ID NO: 56 - protein, 446 aa, Artificial Sequence; identical to SEQ ID NO: 50
  42-TCBcv hole HC

SEQ ID NO: 57 - protein, 216 aa, Artificial Sequence
  42-TCBcv hole HC
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 445 aa, Artificial Sequence
  Mab101 HC-1
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 59 - protein, 670 aa, Artificial Sequence
  Mab101 HC-2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 60 - protein, 216 aa, Artificial Sequence
  Mab101 LC
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 61 - protein, 445 aa, Artificial Sequence
  Mab102 HC-1
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 62 - protein, 670 aa, Artificial Sequence
  Mab102 HC-2
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 63 - protein, 216 aa, Artificial Sequence
  Mab102 LC
EIVLTQSPGTLSLSPGERATLSCRASQSVSSYYLAWYQQKPGQAPRLLISGAGSRATGIPDRFSGSGSGTDFTLTISRLEPEDFAVYYCQQYGYPPDFTFGQGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 64 - protein, 445 aa, Artificial Sequence; identical to SEQ ID NO: 61
  Mab103? HC-1

SEQ ID NO: 65 - protein, 670 aa, Artificial Sequence; identical to SEQ ID NO: 62
  Mab103 HC-2

SEQ ID NO: 66 - protein, 216 aa, Artificial Sequence
  Mab103 LC
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 67 - protein, 13 aa, Artificial Sequence
  83A10 CDR1L (alternative to SEQ ID NO 18)
RASQSVSSSYLAW

SEQ ID NO: 68 - protein, 8 aa, Artificial Sequence
  83A10 CDR2L (alternative to SEQ ID NO 19)
YGASSRAT

SEQ ID NO: 69 - protein, 13 aa, Artificial Sequence
  Mab22 CDR1L (alternative to SEQ ID NO 25)
RASQSVSSYYLAW

SEQ ID NO: 70 - protein, 8 aa, Artificial Sequence
  Mab22 CDR2L (alternative to SEQ ID NO 26)
SGAGSRAT

SEQ ID NO: 71 - protein, 13 aa, Artificial Sequence
  Mab42 CDR1L (alternative to SEQ ID NO 27)
RASQSVSDEYLSW

SEQ ID NO: 72 - protein, 8 aa, Artificial Sequence
  Mab42 CDR2L (alternative to SEQ ID NO 28)
HSASTRAT

(19) United States
(12) Patent Application Publication
(10) Pub. No.: US 2024/0052064 A1
(43) Pub. Date: Feb. 15, 2024

(54) ANTI-BCMA THERAPY IN AUTOIMMUNE DISORDERS

(71) Applicant: BRISTOL-MYERS SQUIBB COMPANY, Princeton, NJ (US)

(72) Inventors: Kofi MENSAH, Medford, MA (US); Robert PLENGE, Wellesley, MA (US); Sophie ROY, Weston, MA (US); Dennis ZALLER, Needham, MA (US); Jennifer DOVEY, Auburndale, MA (US); Steven SAENZ, Boston, MA (US); Jill HENAULT, Acton, MA (US); Camille DOYKAN, North Reading, MA (US); Jenna CALVINO, Brookline, MA (US); Xi JIN, Brookline, MA (US); Joseph PAQUETTE, Seekonk, MA (US)

(21) Appl. No.: 17/798,983

(22) PCT Filed: Feb. 11, 2021
(86) PCT No.: PCT/US21/17665

§ 371 Date: Aug. 14, 2023

Related U.S. Application Data
(60) Provisional application No. 62/975,663, filed on Feb. 12, 2020.

(51) Int. Cl. C07K 16/46; A61P 37/02

(52) U.S. Cl. CPC C07K 16/468; A61P 37/02; C07K 2317/31; C07K 2317/55; C07K 2317/52; C07K 2317/565

(57) ABSTRACT

The present invention relates to the treatment or management of autoimmune disorders, such as autoimmune disorders caused by autoreactive B lineage cells, e.g. anti-neutrophil cytoplasmic antibody (ANCA)-associated vasculitis (AAV).

This application claims priority to U.S. Provisional Application No. 62/975,663, filed on Feb. 12, 2020, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14247-482-228_SEQ_LISTING,” was created on Feb. 11, 2021, and is 106,995 bytes in size.

### FIELD OF THE INVENTION

The present invention relates to the treatment or management of autoimmune disorders, such as autoimmune disorders caused by autoreactive B lineage cells, e.g. anti-neutrophil cytoplasmic antibody (ANCA)-associated vasculitis (AAV).

### BACKGROUND

Autoimmune disorders occur when the immune system of a subject attacks the healthy tissues or organs of the subject's own body. In some cases, these disorders can result from abnormal recognition of antigens on the subject's own tissues (“self-antigens”) by B lineage cells (“autoreactive B lineage cells”), for example memory B cells, plasmablasts and/or plasma cells. In some cases, autoreactive plasmablasts and plasma cells may produce autoreactive antibodies (“autoantibodies”) which recognize the self-antigens and/or attack the healthy tissues or organs expressing the self-antigens. Systemic lupus erythematosus (SLE) has been described as the quintessential autoimmune disorder (Fava, A. and Petri, M. (2019). Systemic lupus erythematosus: Diagnosis and clinical management. Journal of autoimmunity, 96, 1-13).

Anti-neutrophil cytoplasmic antibody (ANCA)-associated vasculitis (AAV) is a serious autoimmune disorder caused by autoreactive B lineage cells that has significant morbidity and mortality. AAV patients cycle between active disease and periods of remission of varying lengths. There is no cure for AAV and 50% of patients die or suffer severe complications during active disease. AAV is characterized by destructive inflammation of small-sized to medium-sized blood vessels mediated by ANCA autoantibodies.

Existing treatments for autoimmune disorders are not always effective in the induction or maintenance of remission and/or may have undesirable side effects. There is therefore a need for further therapies for the treatment or management of autoimmune disorders.

### SUMMARY

The present invention relates to methods of treating or managing a subject having an autoimmune disorder using multispecific (e.g. bispecific) antibodies that bind to BCMA and an antigen that promotes activation of one or more T cells (e.g. CD3).

In one aspect, the present invention provides a method of treating or managing an autoimmune disorder, the method comprising administering to a subject (e.g. a human) in need of such treatment or management a multispecific (e.g. bispecific) antibody, wherein the multispecific antibody binds to BCMA and an antigen that promotes activation of one or more T cells (e.g. CD3).

In another aspect, the present invention provides a multispecific (e.g. bispecific) antibody that binds to BCMA and an antigen that promotes activation of one or more T cells (e.g. CD3) for use in treating or managing an autoimmune disorder in a subject (e.g. a human).

In preferred embodiments, the autoimmune disorder is caused by B lineage cells (e.g. autoreactive B lineage cells). In preferred embodiments, the B lineage cells, e.g. autoreactive B lineage cells, are memory B cells, plasmablasts and/or plasma cells.

In some embodiments, the autoimmune disorder is selected from systemic lupus erythematosus, IgA nephropathy, IgG4 related disease, membranous nephropathy, Myasthenia gravis, Neuromyelitis optica, Pemphigus vulgaris, anti-PAD4-activating rheumatoid arthritis, Sensitized/preformed antibodies in solid organ transplant, Guillain-Barre Syndrome (Acute inflammatory demyelinating polyneuropathy—AIDP), Chronic inflammatory demyelinating polyneuropathy (CIDP), Immune thrombocytopenic purpura, rheumatoid arthritis, and ANCA-associated vasculitis (AAV). In preferred embodiments, the autoimmune disorder is not IgG4-related disease.

Preferably, the autoimmune disorder is ANCA-associated vasculitis (AAV), systemic lupus erythematosus (SLE) and/or rheumatoid arthritis. In preferred embodiments, the autoimmune disorder is ANCA-associated vasculitis (AAV) and/or rheumatoid arthritis.

In some embodiments, the autoimmune disorder is newly diagnosed (e.g. newly diagnosed AAV, SLE or rheumatoid arthritis). In some embodiments, the autoimmune disorder is relapsed or refractory (e.g. relapsed or refractory AAV, SLE or rheumatoid arthritis).

In some embodiments, the AAV comprises diseases that are selected from the group consisting of granulomatosis with polyangiitis (GPA), eosinophilic granulomatosis with polyangiitis (EGPA), microscopic polyangiitis (MPA) and renal-limited ANCA-associated vasculitis. In some embodiments, the AAV is granulomatosis with polyangiitis (Wegener's granulomatosis), eosinophilic granulomatosis with polyangiitis, microscopic polyangiitis or renal-limited ANCA-associated vasculitis.

In some embodiments, the AAV is affecting one or more body parts of the patient selected from nervous system, eyes, nose, heart, kidneys, stomach, intestine, lungs, joints, muscles and skin. In some embodiments, the AAV is generalized with presence of life- or major organ-threatening manifestations, optionally wherein the patient has diffuse alveolar hemorrhage (DAH). In other embodiments, the AAV is localized without organ-threatening manifestations.

In some embodiments, the patient is in need of plasmablast reduction. In some embodiments, the patient is in need of induction of remission. In some embodiments, the patient is in need of maintenance of remission. In some embodiment, the method results in a reduction of plasmablasts in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to no treatment or a reference treatment.

In some embodiments, the patient is in need of induction of remission. In some embodiments, the method is used for the induction of remission, optionally wherein the method results a faster induction of remission in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment. In other embodiments, the patient is in need of maintenance of remission. In some embodiments, the method is used for the maintenance of remission, optionally wherein the method results a longer maintenance of remission in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

In some embodiments, the patient is at risk of developing cytokine release syndrome. In some embodiments, the method results in a lowered incidence of cytokine release syndrome in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

In some embodiments, the patient is at risk of developing infection. In some embodiments, the method results in a lowered incidence of infection in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

In some embodiments, the reference treatment is treatment with steroids (e.g. glucocorticoids), cyclophosphamide, an anti-CD20 monoclonal antibody (e.g. rituximab), methotrexate, azathioprine, mycophenolate, mycophenolate mofetil, avacopan, anti-TNF agents (e.g. infliximab, adalimumab, golimumab, etanercept), anti-IL6R antibodies (e.g. tocilizunab, sarilumab), costimulatory blockade (e.g. abatacept), JAK inhibitors (e.g. tofacitinib, baricitinib) and/or belimumab, preferably wherein the reference treatment is treatment with steroids, cyclophosphamide or rituximab.

In some embodiments, the multispecific (e.g. bispecific) antibody comprises an anti-BCMA antibody, or antigen binding fragment thereof, comprising a CDR1H, CDR2H, CDR3H, CDR1L, CDR2L, and CDR3L region combination selected from the group of:
a) CDR1H region of SEQ ID NO:21, CDR2H region of SEQ ID NO:22, CDR3H region of SEQ ID NO:17, CDR1L region of SEQ ID NO:23, CDR2L region of SEQ ID NO:24, and CDR3L region of SEQ ID NO:20;
b) CDR1H region of SEQ ID NO:21, CDR2H region of SEQ ID NO:22, CDR3H region of SEQ ID NO:17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO:17, CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO:17, CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO:17, CDR1L region of SEQ ID NO:31, CDR2L region of SEQ ID NO:32, and CDR3L region of SEQ ID NO:33; and
g) CDR1H region of SEQ ID NO:15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20.

In particularly preferred embodiments, the anti-BCMA antibody, or antigen binding fragment thereof, comprises a CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20.

In some embodiments, the anti-BCMA antibody, or antigen binding fragment thereof, comprises a VH and a VL selected from the group consisting of:
a) a VH region of SEQ ID NO:10 and a VL region of SEQ ID NO:12,
b) a VH region of SEQ ID NO:10 and a VL region of SEQ ID NO:13,
c) a VH region of SEQ ID NO:10 and a VL region of SEQ ID NO:14,
d) a VH region of SEQ ID NO:38 and a VL region of SEQ ID NO:12,
e) a VH region of SEQ ID NO:39 and a VL region of SEQ ID NO:12,
f) a VH region of SEQ ID NO:40 and a VL region of SEQ ID NO:12, or
g) a VH region of SEQ ID NO:9 and a VL region of SEQ ID NO:11.

In some embodiments, the anti-BCMA antibody, or antigen binding fragment thereof, comprises:
a) a variable region VH comprising an amino acid sequence that is at least 90% identical to, at least 95% identical to, at least 99% identical to, or identical to the amino acid sequence of SEQ ID NO:10 and a variable region VL comprising an amino acid sequence that is at least 90% identical to, at least 95% identical to, at least 99% identical to, or identical to the amino acid sequence of SEQ ID NO:14;
b) a variable region VH comprising an amino acid sequence that is at least 90% identical to, at least 95% identical to, at least 99% identical to, or identical to the amino acid sequence of SEQ ID NO:10 and a variable region VL comprising an amino acid sequence that is at least 90% identical to, at least 95% identical to, at least 99% identical to, or identical to the amino acid sequence of SEQ ID NO:13; or
c) a variable region VH comprising an amino acid sequence that is at least 90% identical to, at least 95% identical to, at least 99% identical to, or identical to the amino acid sequence of SEQ ID NO:9 and a variable region VL comprising an amino acid sequence that is at least 90% identical to, at least 95% identical to, at least 99% identical to, or identical to the amino acid sequence of SEQ ID NO:11.

In particularly preferred embodiments, the anti-BCMA antibody, or antigen binding fragment thereof, comprises a VH region of SEQ ID NO:10 and a VL region of SEQ ID NO: 14.

In some embodiments, the antigen that promotes activation of one or more T cells is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In preferred embodiments, the antigen that promotes activation of one or more T cells is CD3.

In preferred embodiments, the multispecific (e.g. bispecific) antibody comprises an anti-CD3 antibody, or antigen binding fragment thereof, comprising a variable domain VH comprising the heavy chain CDRs of SEQ ID NO: 1, 2 and 3 as respectively heavy chain CDR1H, CDR2H and CDR3H and a variable domain VL comprising the light chain CDRs of SEQ ID NO: 4, 5 and 6 as respectively light chain CDR1L, CDR2L and CDR3L. In some embodiments, the anti-CD3 antibody, or antigen binding fragment thereof, comprises a VH region of SEQ ID NO:7 and a VL region of SEQ ID NO:8.

In some embodiments, the multispecific (e.g. bispecific) antibody comprises an anti-CD3 antibody, or antigen binding fragment thereof, comprising a variable region VH comprising an amino acid sequence that is at least 90% identical to, at least 95% identical to, at least 99% identical to, or identical to the amino acid sequence of SEQ ID NO:7 and a variable region VL comprising an amino acid sequence that is at least 90% identical to, at least 95% identical to, at least 99% identical to, or identical to the amino acid sequence of SEQ ID NO:8.

In particularly preferred embodiments, the multispecific (e.g. bispecific) antibody comprises an anti-BCMA antibody, or antigen binding fragment thereof, comprising a VH region of SEQ ID NO:10 and a VL region of SEQ ID NO: 14, and an anti-CD3 antibody, or antigen binding fragment thereof, comprising a VH region of SEQ ID NO:7 and a VL region of SEQ ID NO:8.

In some embodiments, the multispecific antibody is a bispecific antibody. In preferred embodiments, the multispecific antibody is a bispecific antibody that binds to BCMA and CD3. In some embodiments, the bispecific antibody is bivalent (e.g. the 1+1 format). In some embodiments, the bivalent bispecific antibody has the format: CD3 Fab-BCMA Fab (i.e. when no Fc is present). Alternatively, the bivalent bispecific antibody may have the format: Fc-CD3 Fab-BCMA Fab; Fc-BCMA Fab-CD3 Fab; or BCMA Fab-Fc-CD3 Fab (i.e. when an Fc is present). In preferred embodiments, the bivalent bispecific antibody has the format BCMA Fab-Fc-CD3 Fab. In some embodiments, the bispecific antibody is trivalent (e.g. the 2+1 format). In preferred embodiments, the bispecific antibody is trivalent and comprises two Fab fragments of an anti-BCMA antibody, one Fab fragment of an anti-CD3 antibody, and one Fc portion. In some embodiments, the trivalent bispecific antibody has the format: CD3 Fab-BCMA Fab-BCMA Fab; or BCMA Fab-CD3 Fab-BCMA Fab (i.e. when no Fc is present). Alternatively, the trivalent bispecific antibodies may have the format: BCMA Fab-Fc-CD3 Fab-BCMA Fab; BCMA Fab-Fc-BCMA Fab-CD3 Fab; or CD3 Fab-Fc-BCMA Fab-BCMA Fab (i.e. when an Fc is present). In preferred embodiments, the trivalent bispecific antibody has the format BCMA Fab-Fc-CD3 Fab-BCMA Fab.

In some embodiments, the anti-CD3 Fab comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1.

In some embodiments, the CH1 domain of the anti-BCMA Fab fragment comprises the amino acid modifications K147E/D and K213E/D (numbered according to EU numbering) and a corresponding immunoglobulin light chain comprising a CL domain having amino acid modifications E123K/R/H and Q124K/R/H (numbered according to Kabat).

In alternative embodiments, the CH1 domain of the anti-BCMA Fab fragment comprises the amino acid modifications A141W, L145E, K147T and Q175E (numbered according to EU numbering), or conservative substitutions thereof, and a corresponding immunoglobulin light chain comprising a CL domain having the amino acid modifications F116A, Q124R, L135V and T178R (numbered according to Kabat), or conservative substitutions thereof.

In some embodiments, the multispecific (e.g. bispecific) antibody further comprises an Fc. In some embodiments, the Fc is an IgG1 Fc. In some embodiments, the (e.g. IgG1) Fc comprises a first Fc chain comprising first constant domains CH2 and CH3, and a second Fc chain comprising second constant domains CH2 and CH3, wherein:
a) the first CH3 domain comprises the modifications T366S, L368A and Y407V, or conservative substitutions thereof (numbered according to EU numbering); and
b) the second CH3 domain comprises the modifications T366W, or conservative substitutions thereof (numbered according to EU numbering).

In alternative embodiments, the (e.g. IgG1) Fc comprises a first Fc chain comprising first constant domains CH2 and CH3, and a second Fc chain comprising second constant domains CH2 and CH3, wherein:
a) the first CH3 domain comprises the modifications T350V, L351Y, F405A and Y407V, or conservative substitutions thereof (numbered according to EU numbering); and
b) a second CH3 domain comprising the modifications T350V, T366L, K392L and T394W, or conservative substitutions thereof (numbered according to EU numbering).

In some embodiments, the (e.g. IgG1) Fc comprises:
a) the modifications L234A, L235A and P329G (numbered according to EU numbering); and/or
b) the modifications D356E, and L358M (numbered according to EU numbering).

In further embodiments, the multispecific (e.g. bispecific) antibody according to the invention comprises the following SEQ ID NOs:
i. 83A10-TCBcv: 45, 46, 47 (×2), 48
ii. 21-TCBcv: 49, 50, 51 (×2), 48
iii. 22-TCBcv: 52, 53, 54 (×2), 48
iv. 42-TCBcv: 55, 56, 57 (×2), 48
v. Mab101: 58, 59, 60 (×2), 48
vi. Mab102: 61, 62, 63 (×2), 48
vii. Mab103: 64, 65, 66 (×2), 48

In preferred embodiments, the bispecific antibody according to the invention is 42-TCBcv, Mab101 or Mab102. In particularly preferred embodiments, the bispecific antibody according to the invention is 42-TCBcv.

Aspects and embodiments of the invention are set out in the appended claims. These and other aspects and embodiments of the invention are also described herein.

### DESCRIPTION OF FIGURES

The present invention will now be described in more detail with reference to the attached Figures, in which:

FIG. 1 illustrates different formats of bispecific bivalent antibodies for use in the present invention, which comprise Fab fragments binding to a T cell antigen (CD3 is illustrated) and BCMA in the format Fab BCMA-Fc-Fab CD3. The CD3 Fab may include a VH-VL crossover to reduce light chain mispairing and side-products. Amino acid substitutions (“RK/EE” are illustrated) may be introduced in CL-CH1 to reduce light chain mispairing/side products in production. The CD3 Fab and BCMA Fab may be linked to each other with flexible linkers.

FIG. 2 illustrates different formats of bispecific trivalent antibodies for use in the present invention, which comprise Fab fragments binding to a T cell antigen (CD3 is illustrated) and BCMA in the following formats: Fab BCMA-Fc-Fab CD3-Fab BCMA (AB); Fab BCMA-Fc-Fab BCMA-Fab CD3 (C,D). The CD3 Fab may include a VH-VL crossover to reduce light chain mispairing and side-products. Amino acid substitutions (“RK/EE” are illustrated) may be introduced in CL-CH1 to reduce light chain mispairing/side products in production. The CD3 Fab and BCMA Fab may be linked to each other with flexible linkers.

FIG. 3 illustrates further formats of bispecific bivalent antibodies for use in the present invention, which comprise Fab fragments binding to a T cell antigen (CD3 is illustrated) and BCMA in the following formats: Fc-Fab CD3-Fab BCMA (A,B); Fc-Fab BCMA-Fab CD3 (C,D). The CD3 Fab may include a VH-VL crossover to reduce light chain mispairing and side-products. Amino acid substitutions (“RK/EE” are illustrated) may be introduced in CL-CH1 to reduce light chain mispairing/side products in production. The CD3 Fab and BCMA Fab may be linked to each other with flexible linkers.

FIG. 4 illustrates BMCA expression on plasmablasts (PB) from four normal healthy volunteers (NHV) compared to BCMA-expressing cancer cell lines (JEKO, RPMI-8226 and H929), as assessed by flow cytometry (A). Soluble BCMA levels are shown in serum or plasma samples from NHV (‘Normal’), Multiple Myeloma (‘MM’) or ANCA-Associated Vasculitis (‘AAV’) patients (B), as assessed by ELISA.

FIG. 5 illustrates dose-response curves of T cell-mediated killing (A) and T cell activation (B) when JEKO cells were cultured with CD3+ T cells at a 1:2 target:effector (T:E) and treated with anti-BCMA anti-CD3 bispecific antibodies, i.e. BCMA T cell engagers, (CC-93269, Mab101 or Mab102). T cell-mediated killing of JEKO cells was assessed by annexinV expression; the 20 hour time point is shown. T cell lineage and activation markers were analyzed by flow cytometry at the 24 hour time point; % CD69 expression on CD8+ T cells is shown.

FIG. 6 illustrates dose-response curves of T cell-mediated killing (A) and T cell activation (B) when RPMI-8226 cells were co-cultured with NHV PBMCs at different target:effector (T:E) ratios and various concentrations of CC-93269.

FIG. 7 illustrates dose-response curves for plasmablast killing (A), T cell activation (C) and cytokine production (D) when peripheral blood mononuclear cells (PBMC) from healthy volunteers were treated with various concentrations of BCMA T cell engagers, i.e. BCMA TCE (Mab101, CC-93269 or Mab102) or control 2+1 antibody for 24 hours. Representative FACS plot shows plasmablast identification as CD20(−) CD27(+), gated on CD3(−) CD19(+) cells (B). Plasmablast killing is assessed as percent of total CD19(+) cells (A). % CD69 expression on CD8+ T cells is shown (C).

FIG. 8 illustrates dose-response curves for cytokine production (IFNγ, IL-6, IL-2, IL-10, granzyme B and perforin) when PBMC from healthy volunteers were treated with various concentrations of CC-93269 for 24 hours.

FIG. 9 illustrates B cell lineage when PBMC from healthy volunteers were treated with various concentrations of CC-93269 for 24 hours. Naïve B cells with CD20(+)CD27(−)IgD(+) expression (A), unswitched memory B cells with CD20(+)CD27(+)IgD(+) expression (B) and switched memory B cells with CD20(+)CD27(+)IgD(−) expression (C) are given as a percent of total CD19(+) cells.

FIG. 10 illustrates dose-response curves for plasmablast killing (A) and T cell activation (B) when bone marrow (BM) mononuclear cells were treated with CC-93269 for 24 hours, as compared to PBMC suspended in media or PBMC suspended in bone marrow (BM) supernatant. Plasmablast killing is assessed as percent of total CD19(+) cells (A). % CD69 expression on CD8+ T cells is shown (B).

FIG. 11 illustrates dose-response curves for plasmablast killing (A), T cell activation (C) and cytokine production (D) when PBMC from AAV patients were treated with various concentrations of BCMA TCE (Mab101, CC-93269 or Mab102) or control 2+1 antibody for 24 hours. Representative FACS plot shows plasmablast identification as CD20(−) CD27(+), gated on CD3(−) CD19(+) cells (B). Plasmablast killing is assessed as percent of total CD19(+) cells (A). % CD69 expression on CD8+ T cells is shown (C).

FIG. 12 illustrates T cell activation in PBMCs from an AAV patient, AAV1, when incubated with BCMA TCE (Mab101 [A], CC-93269 [B] or Mab102 [C]) for 24 hours as assessed by staining for T cell lineage (CD4, CD8) and activation markers (CD69, CD25). % CD69 or % CD25 expression levels on CD4+ cells or CD8+ cells are shown.

FIG. 13 illustrates selective plasmablast (PB) depletion in PBMCs from an AAV patient, AAV-5, who had last received rituximab 5 months prior, after incubation with various concentrations of CC-93269 (B, C) or control 2+1 antibody (A).

FIG. 14 illustrates CD19(+) CD20(−) CD27(+) plasmablast targets (A) and CD4(+)/CD8(+) T cells in AAV-2 subject at baseline (A). PBMCs from AAV-2 were incubated with various concentrations of BCMA TCE and following 24 hour incubation, T cell activation was assessed by flow cytometry (C).

FIG. 15 illustrates dose-response curves of T cell activation when JEKO-1 cells at different T:E ratios were incubated with CC-93269 or control 2+1 antibody. JEKO-1 cells were cultured with PBMC at T:E ratios of 1:10 or 1:500 to mimic T:E ratios observed in Multiple Myeloma (MM) or AAV, respectively. Following 24 hour incubation, cells were washed and then CD69 (A) and CD25 (B) expression on CD8(+) T cells was assessed.

FIG. 16 illustrates plasmablast levels (A) and total IgG secretion (B) when PBMCs from healthy volunteers were incubated with various concentrations of BCMA TCE for 24 hours prior to stimulation with ODN2006 (CpG, 10 μg/mL) or cultured with growth factors IL-2 (20 U/ml) BAFF (200 ng/ml) and IL-21 (100 ng/ml) for 4-7 days.

FIG. 17 illustrates dose-response curves for plasmablast killing (A), T cell activation (B) and cytokine production (C) when PBMC from Rheumatoid Arthritis (RA) were treated with various concentrations of CC-93269 or control 2+1 antibody for 24 hours.

FIG. 18 illustrates B cell lineage of PBMC from RA after incubation with various concentrations of CC-93269 for 24 hours. Naïve B cells with CD20(+)CD27(−)IgD(+) expression (A), unswitched memory B cells with CD20(+)CD27(+)IgD(+) expression (B) and switched memory B cells with CD20(+)CD27(+)IgD(−) expression (C) are given as a percent of total CD19(+) cells.

FIG. 19 illustrates selective IRF4+ plasmablast (PB) depletion in PBMCs from cynomolgus macaque, after incubation with various concentrations of CC-93269 or control 2+1 antibody (A), with minimal CD20(+) B cell depletion (B) and minimal elevation in the frequency of activated CD69(+)CD8(+) T cells (C).

FIG. 20 illustrates dose-response curves for plasmablast killing (A) and T cell activation (B) when PBMC from systemic lupus erythematosus (SLE) patients (n=5) were treated with various concentrations of CC-93269 for 24 hours.

FIG. 21 illustrates the effect of exogenous soluble BCMA (sBCMA) on plasmablast killing (A) and T cell activation (B) when PBMC from normal healthy volunteers were treated with CC-93269 for 24 hours.

FIG. 22 illustrates dose-response curves for T cell activation (A) and cytokine production (B) when whole blood samples from normal healthy volunteers (n=4) or AAV patients (n=2) were treated with various concentrations of CC-93269 for 24 hours.

FIG. 23 illustrates representative SEC chromatogram overlays of the 22-TCBcv molecule following storage in the conditions specified in Example 18.3.3.

FIG. 24 illustrates thermal unfolding of bispecific antibodies Mab101, Mab102, 83A10-TCBcv and 22-TCBcv. All bispecific antibodies exhibited similar thermal unfolding onset temperatures (−60° C.). However, the Tmapp values for the largest transition were roughly 5° C. greater for the Mab101 and 83A10-TCBcv molecules comprising common CDR regions.

FIG. 25 illustrates a colloidal stability assessment of bispecific antibodies Mab101, Mab102, 83A10-TCBcv and 22-TCBcv. Assessment was carried out by PEG 6000 precipitation and was performed in pH 6 buffer. The molecules comprising the CDR regions of 83A10 (i.e. Mab101 and 83A10-TCBcv) required nearly twice as much PEG 6000 to induce native state precipitation by excluded volume effects. Solid lines are fitted to Equation 1.

FIG. 26 illustrates a representative single cycle kinetics SPR sensorgram of the bispecific antibody 83A10-TCBcv. The antibody was buffered at pH 6 and stored for two weeks at 2-8° C. (A) or buffered at pH 8 and stored for two weeks at 40° C. (B). The dashed line represents the average of 10 independent measurements of the non-stressed sample prior to storage and the dotted lines indicate 3 standard deviations (SD) of this average. Percent similarity scores were calculated using the number of data points of the stressed samples that fall within the SD window according to Equation 2.

FIG. 27 is a sequence alignment of the bispecific antibodies Mab101, Mab102, 83A10-TCBcv, and 22-TCBcv. The CDR regions are shaded and the percent sequence identity is shown above.

### DETAILED DESCRIPTION

As used herein, the articles “a” and “an” may refer to one or to more than one (e.g. to at least one) of the grammatical object of the article.

“About” may generally mean an acceptable degree of error for the quantity measured given the nature or precision of the measurements. Exemplary degrees of error are within 20 percent (%), typically, within 10%, and more typically, within 5% of a given value or range of values.

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Therapeutic Methods

The invention is based, at least in part, on the treatment or management of a subject having an autoimmune disorder with multispecific (e.g. bispecific) antibodies that bind to an antigen that promotes activation of one or more T cells (e.g. CD3) and BCMA.

In certain embodiments, the “subject” or “patient” is a human. In some embodiments, the “subject” or “patient” is less than 18 years old. In some embodiments, the “subject” or “patient” is 18 years old or older. In some embodiments, the subject is in need of induction of remission. In some embodiments, the subject is in need of maintenance of remission. In some embodiments, the patient is in need of plasmablast reduction.

As used herein, the terms “treat,” “treating” or “treatment,” and the like refer to obtaining a desired pharmacologic and/or physiologic effect. Preferably, the effect is therapeutic, i.e., the effect partially or completely cures a disease and/or adverse symptom attributable to the disease.

Alternatively, the pharmacologic and/or physiologic effect may be prophylactic, i.e., the effect completely or partially prevents a disease or symptom thereof.

As used herein, the terms “manage”, “managing” or “management”, and the like refer to suppressing and/or delaying the progression and/or worsening of a disease and/or adverse symptoms attributable to the disease.

The present invention relates to the treatment or management of an autoimmune disorder with multispecific (e.g. bispecific) antibodies that bind to an antigen that promotes activation of one or more T cells (e.g. CD3) and BCMA.

In some embodiments, the multispecific (e.g. bispecific) antibodies of the invention are capable of selectively binding to the cells causing the autoimmune disorder, for example to BCMA-expressing cells causing the autoimmune disorder.

In preferred embodiments, the autoimmune disorder is caused by B lineage cells (e.g. BCMA-expressing B lineage cells). In some embodiments, the B lineage cells (e.g. BCMA-expressing B lineage cells) are autoreactive B lineage cells. In some embodiments, the B lineage cells (e.g.

BCMA-expressing B lineage cells) drive autoimmunity e.g. by serving as antigen presenting cells or by secretion of proinflammatory cytokines.

As used herein, the term “autoreactive B lineage cells” refers to B lineage cells capable of recognizing antigens on the subject's own tissues (“self-antigens”). The autoreactive B lineage cells may be antibody-secreting cells and/or may be capable of secreting antibodies. In some embodiments, the autoreactive B lineage cells are plasmablasts, plasma cells, memory B cells, or any combination thereof. In preferred embodiments, the autoreactive B lineage cells are plasmablasts, plasma cells and memory B cells. In particularly preferred embodiments, the autoreactive B lineage cells are plasmablasts.

In some embodiments, the multispecific (e.g. bispecific) antibodies of the invention are capable of selectively binding to the autoreactive B lineage cells causing the autoimmune disorder. In preferred embodiments, the autoreactive B lineage cells are BCMA-expressing cells such as memory B cells, plasmablasts and/or plasma cells.

Plasmablasts are precursors of plasma cells. They may be identified by a combination of one or more of the markers selected from CD19(+), CD20(−), CD27(+), CD38(+), BCMA(+), IgD(−), CD24(−), SLAMF7(+), and/or CD138(−). In some embodiments, plasmablasts are identified as cells displaying BCMA(+) and SLAMF7(+), optionally wherein the cells also display one or more of the markers IgD(−), CD38(+), and/or CD138(−). In some embodiments, plasmablasts are identified as cells displaying CD19(+) CD20(−) CD27(+) BCMA(+) SLAMF7(+) IgD(−) CD38(+) CD138(−). In particularly preferred embodiments, plasmablasts are identified as cells displaying the markers CD19(+) CD20(−) CD27(+), optionally wherein the cells also display one or more of the markers BCMA(+) and/or CD38(+).

Plasma cells are antibody-secreting cells. As used herein, the term “plasma cells” may refer to short-lived and/or long-lived plasma cells. They may be identified by a combination of one or more of the markers selected from CD19(+), CD20(−), CD27(+), CD38(+), BCMA(+), IgD(−), CD138(+) CD24(−) and/or SLAMF7(+). In some embodiments, plasma cells are identified as cells displaying the markers BCMA(+) SLAMF7(+) CD138(+), optionally wherein the cells also display one or more of the markers IgD(−) and/or CD38(+). In some embodiments, plasma cells are identified as cells displaying CD19(+) CD20(−) CD27(+) BCMA(+) SLAMF7(+) IgD(−) CD38(+) CD138(+).

In particularly preferred embodiments, plasma cells are identified as cells displaying the markers CD19(+) CD20(−) CD27(+), optionally wherein the cells also display one or more of the markers BCMA(+) CD38(+) and/or CD138(+).

Memory B cells are B cells activated by antigens and T-cell helpers in extrafollicular or GC reactions. They may be identified by a combination of one or more of the markers selected from CD19(+), CD20(+), CD27(+), CD38(−), BCMA(+/−), IgD(−) and/or CD24(+). In some embodiments, memory B cells are identified as cells displaying the markers IgD(−) CD38(−) BCMA(+/−). In some embodiments, memory B cells are identified as cells displaying the markers CD19 (+) CD20 (+) CD27 (+) IgD (−) CD38 (−) BCMA (+/−). In particularly preferred embodiments, memory B cells are identified as cells displaying the markers CD19(+) CD20(+) CD27(+), optionally wherein the cells also display one or more of the markers selected from BCMA(+) and/or CD38(−).

The present inventors have identified that the multispecific (e.g. bispecific) antibodies of the invention may be used in the treatment or management of a patient having an autoimmune disorder, wherein a blood sample from the patient has an amount of soluble BCMA of less than the amount of soluble BCMA in multiple myeloma (MM) patients and/or an amount of soluble BCMA comparable to the amount of soluble BCMA in normal healthy patients. Soluble BCMA in a blood sample (e.g. isolated serum or plasma) from the patient may be measured by ELISA, for example using bead-based immunoassay by Ampersand Biosciences (Lake Clear, NY).

In some embodiments, a blood sample from the patient having an autoimmune disorder has an amount of soluble BCMA of less than the amount of soluble BCMA in patients having a B cell malignancy (e.g. a BCMA-expressing cancer). In some embodiments, a blood sample from the patient having an autoimmune disorder has an amount of soluble BCMA of less than the amount of soluble BCMA in MM patients, preferably about 1.5-fold less, about 2-fold less, about 2.5-fold less, about 3-fold less, about 3.5-fold less, about 4-fold less, about 4.5-fold less, about 5-fold less, about 5.5-fold less, or about 6-fold less than the amount of soluble BCMA in MM patients. In some embodiments, a blood sample from the patient having an autoimmune disorder has a soluble BCMA of less than about 150 ng/ml, less than about 100 ng/ml, less than about 75 ng/ml, less than about 50 ng/ml, less than about 40 ng/ml, less than about 35 ng/ml, or less than about 30 ng/ml as measured by ELISA. In some embodiments, a blood sample from the patient having an autoimmune disorder has an amount of soluble BCMA within about 40 ng/ml, within about 30 ng/ml, within about 20 ng/ml, within about 15 ng/ml, within about 10 ng/ml, or within about 5 ng/ml of the amount of soluble BCMA in a normal healthy person as measured by ELISA. In some embodiments, a blood sample from the patient having an autoimmune disorder has an amount of soluble BCMA of at least 5 ng/ml, at least 7.5 ng/ml, at least 10 ng/ml, at least 12.5 ng/ml, or at least 15 ng/ml as measured by ELISA. In some embodiments, a blood sample from the patient having an autoimmune disorder has an amount of soluble BCMA of between about 5 ng/ml and 50 ng/ml, between about 5 ng/ml and 40 ng/ml, between about 5 ng/ml and 30 ng/ml, between about 10 ng/ml and 50 ng/ml, between about 10 ng/ml and 40 ng/ml, or between about 10 ng/ml and 30 ng/ml, as measured by ELISA. Soluble BCMA in a blood sample (e.g. isolated serum or plasma) from the patient as measured by ELISA may be measured using bead-based immunoassay by Ampersand Biosciences (Lake Clear, NY). The present inventors have identified that the multispecific (e.g. bispecific) antibodies of the invention can be used in the treatment or management of a patient having an autoimmune disorder caused by plasmablasts and/or plasma cells, wherein the patient has a plasmablast BCMA surface receptor density comparable to the plasmablast BCMA surface receptor density of a normal healthy volunteer.

In some embodiments, the patient in need of treatment or management of an autoimmune disorder has a BCMA surface receptor density on plasmablasts of less than about 10,000 molecules, less than about 5000 molecules, less than about 2500 molecules, or less than about 2000 molecules, as measured using a flow cytometry system based on a standard curve generated with anti-BCMA antibody coated beads.

In some embodiments, the patient in need of treatment or management of an autoimmune disorder has a BCMA surface receptor density on plasmablasts of at least about 500 molecules, at least about 700 molecules, at least about 900 molecules or at least about 1000 molecules, as measured using a flow cytometry system based on a standard curve generated with anti-BCMA antibody coated beads.

In some embodiments, the patient in need of treatment or management of an autoimmune disorder has a BCMA surface receptor density on plasmablasts of between about 800 and about 2200 molecules, between about 900 and 2100 molecules, or between about 1000 and 2000 molecules using a flow cytometry system based on a standard curve generated with anti-BCMA antibody coated beads.

In some embodiments, the method for treatment or management of the present invention results in a reduction of plasmablasts in the patient by at least 5%, 10%, 15%, 20%, 25%, 30%, 35%, 40%, 45%, 50%, 55%, 60%, 65%, 70%, 75%, 80%, 85%, 90%, or 95%, or 100% relative to no treatment or a reference treatment. In preferred embodiments, the method for treatment or management of the present invention results in a reduction of plasmablasts in the patient by at least 75%. In particularly preferred embodiments, the method for treatment or management of the present invention results in a reduction of plasmablasts in the patient by at least 90%.

In some embodiments, the method for treatment or management of the present invention results in a reduction of plasma cells in the patient by at least 5%, 10%, 15%, 20%, 25%, 30%, 35%, 40%, 45%, 50%, 55%, 60%, 65%, 70%, 75%, 80%, 85%, 90%, or 95%, or 100% relative to no treatment or a reference treatment. In preferred embodiments the method for treatment or management of the present invention results in a reduction of plasma cells in the patient by at least 75%. In particularly preferred embodiments, the method for treatment or management of the present invention results in a reduction of plasma cells in the patient by at least 90%.

In some embodiments, the method for treatment or management of the present invention results in a reduction of plasma cells and plasmablasts in the patient by at least 5%, 10%, 15%, 20%, 25%, 30%, 35%, 40%, 45%, 50%, 55%, 60%, 65%, 70%, 75%, 80%, 85%, 90%, or 95%, or 100% relative to no treatment or a reference treatment. In preferred embodiments the method for treatment or management of the present invention results in a reduction of plasma cells and plasmablasts in the patient by at least 75%. In particularly preferred embodiments, the method for treatment or management of the present invention results in a reduction of plasma cells and plasmablasts in the patient by at least 90%.

Existing treatments for autoimmune disorders such as AAV, SLE or RA include cyclophosphamide and/or an anti-CD20 monoclonal antibody (e.g. rituximab) with high doses of steroids (e.g. glucocorticoids). As used herein, the term “reference treatment” (e.g. for AAV) preferably refers to treatment with cyclophosphamide, rituximab and/or steroids. Alternatively or in addition, “reference treatment” (e.g. SLE or RA) refers to treatment with anti-TNF agents (e.g. infliximab, adalimumab, golimumab, etanercept), anti-IL6R antibodies (e.g. tocilizumab, sarilumab), costimulatory blockade (e.g. abatacept), JAK inhibitors (e.g. tofacitinib, baricitinib) and/or belimumab. Additional treatments include cyclophosphamide, methotrexate, azathioprine, mycophenolate, mycophenolate mofetil and/or avacopan. However, such existing treatments are not always effective and/or durable. Moreover, they can have adverse side effects.

Without being bound by theory, the present invention is anticipated to selectively deplete the B-lineage cells causing the autoimmune disorder e.g. autoreactive B-lineage cells, e.g. plasmablasts, plasma cells and memory B cells. The method for treatment or management of the present invention may therefore result in a faster induction of remission and/or fewer off-target effects are anticipated relative to a reference treatment which does not selectively deplete the B-lineage cells causing the autoimmune disorder.

In some embodiments, the method of treatment is used for the induction of remission. In some embodiments, the method for treatment or management of the present invention results in a faster induction of remission in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

The method for treatment or management of the present invention may result in a better maintenance of remission relative to a reference treatment. For example, following the depletion of plasmablasts and plasma cells, the multispecific (e.g. bispecific) antibodies of the invention may suppress and/or delay the recovery of plasmablasts and plasma cells (e.g. as measured by FACS and/or IgG secretion) from BCMA negative precursors even after incubation with growth factors for their regeneration. Additionally, following the depletion of plasmablasts and plasma cells, the multispecific (e.g. bispecific) antibodies of the invention may suppress and/or delay the production of antibodies, e.g. autoantibodies causing autoimmune disorders even after stimulation with CpG.

In some embodiments, the multispecific (e.g. bispecific) antibodies of the invention suppress and/or delay the production of IgG antibodies, despite stimulation with IL-2, BAFF, and IL-21 or CpG (ODN2006) to induce plasmablast/plasma cell differentiation. The multispecific (e.g. bispecific) antibodies of the invention may be incubated with isolated peripheral blood mononuclear cells (PBMC) at the EC50 concentration for plasmablast lysis for 24 hours prior to stimulation with CpG (ODN2006 10 μg/mL), IL-2 (20 U/ml), BAFF (200 ng/ml) and IL-21 (100 ng/ml) for 7 days, after which IgG concentrations may be less than about 4000 pg/ml, less than about 3500 pg/ml, less than about 3000 pg/ml, less than about 2500 pg/ml, or less than about 2000 pg/ml, as measured by ELISA. The multispecific (e.g. bispecific) antibodies of the invention may alternatively be incubated with isolated peripheral blood mononuclear cells (PBMC) at the EC90 concentration for plasmablast lysis for 24 hours prior to stimulation with CpG (ODN2006 10 μg/mL)), IL-2 (20 U/ml), BAFF (200 ng/ml) and IL-21 (100 ng/ml) for 7 days, after which IgG concentrations may be less than about 2000 pg/ml, less than about 1800 pg/ml, less than about 1000 pg/ml, or less than about 500 pg/ml as measured by ELISA.

In some embodiments, the method of treatment is used for the induction and maintenance of remission. In some embodiments, the method is used for the maintenance of remission. In some embodiments, the method for treatment or management of the present invention results in a longer maintenance of remission in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

In some embodiments, the autoimmune disorder is relapsed or refractory. As used herein, the term “relapsed” is intended to mean the return of the disorder or the signs and symptoms of the disorder after a period of improvement. As used herein, the term “refractory” is intended to mean that the particular disorder is resistant to, or non-responsive to, therapy with a particular therapeutic agent.

A disorder can be refractory to therapy with a particular therapeutic agent either from the onset of treatment with the particular therapeutic agent (i.e., non-responsive to initial exposure to the therapeutic agent), or as a result of developing resistance to the therapeutic agent, either over the course of a first treatment period with the therapeutic agent or during a subsequent treatment period with the therapeutic agent.

In some embodiments, the autoimmune disorder is relapsed or refractory to cyclophosphamide, anti-CD20 monoclonal antibodies (e.g. rituximab), glucocorticoids (e.g. methylprednisolone, dexamethasone), antifolates (e.g. methotrexate), inhibitors of purine synthesis (e.g. azathioprine, mycophenolate and/or mycophenolate mofetil), C5a inhibitors (e.g. avacopan), anti-CD19 antibodies, BAFF/APRIL antagonists, proteasome inhibitors (e.g. bortezomib), anti-CD22 monoclonal antibodies, anti-TNF agents (e.g. infliximab, adalimumab, golimumab, etanercept), anti-IL6R antibodies (e.g. tocilizunab, sarilumab), costimulatory blockade (e.g. abatacept), JAK inhibitors (e.g. tofacitinib, baricitinib), belimumab. and/or Bruton's tyrosine kinase (BTK) inhibitors.

In alternative embodiments, the autoimmune disorder is newly diagnosed.

Autoimmune disorders amenable to treatment with the multispecific (e.g. bispecific) antibodies of the invention include, without limitation, achalasia, Addison's disease, acute inflammatory demyelinating polyneuropathy—AIDP, adult Still's disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN), Balb disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis, dermatitis herpetiformis, dermatomyositis, Devic's disease (neuromyelitis optica), diabetes mellitus, discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture's syndrome, granulomatosis with polyangiitis, Graves' disease, Guillain-Barre syndrome, Hashimoto's disease, Hashimoto's thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura, IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and II, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized/preformed antibodies in solid organ transplant, Sjögren's syndrome, sperm & testicular autoimmunity, stiff person syndrome (SPS), systemic lupus erythematosus (SLE), subacute bacterial endocarditis (SBE), Susac's syndrome, sympathetic ophthalmia (SO), Takayasu's arteritis, temporal arteritis/Giant cell arteritis, thrombocytopenic purpura, thrombotic thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener's disease. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis).

In some embodiments, the multispecific (e.g. bispecific) antibodies of the invention treat or manage AAV. In some embodiments, the multispecific (e.g. bispecific) antibodies of the invention treat or manage rheumatoid arthritis. In some embodiments, the multispecific (e.g. bispecific) antibodies of the invention treat or manage SLE. In some embodiments, the multispecific (e.g. bispecific) antibodies of the invention treat or manage AAV and rheumatoid arthritis. In some embodiments, the multispecific (e.g. bispecific) antibodies of the invention treat or manage AAV, SLE and rheumatoid arthritis.

In one aspect, the present invention provides a method of treating or managing AAV, the method comprising administering to a subject (e.g. a human) in need of such treatment or management a multispecific (e.g. bispecific) antibody, wherein the multispecific antibody binds to BCMA and an antigen that promotes activation of one or more T cells (e.g. CD3).

In another aspect, the present invention provides a multispecific (e.g. bispecific) antibody that binds to BCMA and an antigen that promotes activation of one or more T cells (e.g. CD3) for use in treating or managing AAV in a subject (e.g. a human).

In one aspect, the present invention provides a method of treating or managing rheumatoid arthritis (e.g. relapsed or refractory rheumatoid arthritis), the method comprising administering to a subject (e.g. a human) in need of such treatment or management a multispecific (e.g. bispecific) antibody, wherein the multispecific antibody binds to BCMA and an antigen that promotes activation of one or more T cells (e.g. CD3).

In another aspect, the present invention provides a multispecific (e.g. bispecific) antibody that binds to BCMA and an antigen that promotes activation of one or more T cells (e.g. CD3) for use in treating or managing rheumatoid arthritis (e.g. relapsed or refractory rheumatoid arthritis) in a subject (e.g. a human).

In one aspect, the present invention provides a method of treating or managing SLE (e.g. relapsed or refractory SLE), the method comprising administering to a subject (e.g. a human) in need of such treatment or management a multispecific (e.g. bispecific) antibody, wherein the multispecific antibody binds to BCMA and an antigen that promotes activation of one or more T cells (e.g. CD3).

In another aspect, the present invention provides a multispecific (e.g. bispecific) antibody that binds to BCMA and an antigen that promotes activation of one or more T cells (e.g. CD3) for use in treating or managing SLE (e.g. relapsed or refractory SLE) in a subject (e.g. a human).

T-Cell Mediated Lysis, T-Cell Activation and Cytokine Production

The multispecific (e.g. bispecific) antibodies of the invention bind to an antigen that promotes activation of one or more T cells (e.g. CD3). As used herein, the term “T cell antigen” refers to an antigen that promotes activation of one or more T cells (e.g. CD3).

In preferred embodiments, the T cell antigen (e.g. CD3) is a human T cell antigen (e.g. human CD3). In preferred embodiments, the T cell antigen is CD3.

Thus, binding of the multispecific (e.g. bispecific) antibodies of the invention to a T cell antigen (e.g. CD3) may allow for recruitment of the T cell to the BCMA-expressing cell to result in lysis of the BCMA-expressing cell (e.g. plasmablast, plasma cell, and/or memory B cell). The multispecific (e.g. bispecific) antibodies of the invention are therefore capable of inducing selective depletion of BCMA-expressing cells (e.g. plasmablast, plasma cell and/or memory B cell) by redirecting cytotoxic T cells to the BCMA-expressing cells.

The present inventors have identified that the multispecific (e.g. bispecific) antibodies of the invention can be administered at a lower dose in the treatment of disorders characterized by a low ratio of target BCMA expressing cells to effector T cells (T:E ratio), e.g. autoimmune disorders, than would be required for the treatment of diseases characterized by a high T:E ratio, e.g. B cell malignancies such as multiple myeloma.

Accordingly, in some embodiments, the multispecific (e.g. bispecific) antibodies of the invention are administered to a subject having an autoimmune disorder, wherein the individual has a ratio of BCMA expressing cells to effector T cells of less than about 1:15, less than about 1:30, less than about 1:50, less than about 1:100, or less than 1:500. The ratio of BCMA expressing cells to effector T cells (T:E ratio) may be measured in an isolated body fluid sample from the subject having an autoimmune disorder, for example in a blood sample, bone marrow aspirate or synovial fluid from the subject having an autoimmune disorder.

The present inventors have identified that the multispecific (e.g. bispecific) antibodies of the invention can achieve a therapeutic effect at a lower dose in the treatment of the autoimmune disorders disclosed herein (e.g. autoimmune disorders caused by BCMA-expressing B lineage cells) than would be required for the treatment of B cell malignancies (e.g. BCMA-expressing cancers such as multiple myeloma). In particular, the multispecific (e.g. bispecific) antibodies of the invention may achieve lysis of the pathogenic cells (e.g. the BCMA-expressing autoreactive B lineage cells) of the autoimmune disorder at a lower dose (e.g. between 10-fold and 100-fold lower) than the dose required for lysis of the pathogenic cells (e.g. the BCMA-expressing malignant cells) of multiple myeloma.

Thus, in one aspect, the present invention provides a method for treatment or management of an autoimmune disorder with multispecific (e.g. bispecific) antibodies that bind to an antigen that promotes activation of one or more T cells (e.g. CD3) and BCMA, wherein the treatment comprises the administration of the multispecific (e.g. bispecific) antibody at a dose of between about 0.01 mg and about 1 mg. In embodiments in which the multispecific (e.g. bispecific) antibody is the CC-93269 antibody disclosed herein, the dose may be between about 0.01 mg and about 1 mg.

In some embodiments of any of the aspects disclosed herein, the treatment comprises at least one dose of the multispecific (e.g. bispecific) antibody, e.g. at least two or at least three doses of the multispecific (e.g. bispecific) antibody. In alternative embodiments, up to three doses of the multispecific (e.g. bispecific) antibody, e.g. up to two or a single dose of the multispecific (e.g. bispecific) antibody is administered. In preferred embodiments, a single dose of the multispecific (e.g. bispecific) antibody is administered.

In particularly preferred embodiments, the treatment comprises a single dose of the multispecific (e.g. bispecific) antibody at a dose of between about 0.01 mg and about 1 mg.

In some embodiments, the multispecific (e.g. bispecific) antibodies of the invention achieve plasmablast lysis when incubated with isolated PBMC (e.g. isolated PBMC from a patient with an autoimmune disorder) for 24 hours wherein the multispecific (e.g. bispecific) antibodies of the invention are at a concentration lower than needed to kill BCMA-expressing cancer cells (e.g.

JEKO-1 cell line or MM cell line). PBMC can be isolated from whole blood samples, for example using Ficoll gradient resuspended in RPMI+10% HI FBS. In some embodiments, the multispecific (e.g. bispecific) antibodies of the invention achieve plasmablast lysis when incubated with whole blood (e.g. a whole blood sample from a patient with an autoimmune disorder) for 24 hours wherein the multispecific (e.g. bispecific) antibodies of the invention are at a concentration lower than needed to kill BCMA-expressing cancer cells (e.g. JEKO-1 cell line or MM cell line). Plasmablast depletion is measured by FACS whereby plasmablasts are identified as CD19(+) CD20(−) CD27(+) cells. In some embodiments, the multispecific (e.g. bispecific) antibodies of the invention may achieve lysis of plasmablasts when incubated with isolated PBMC (e.g. isolated PBMC from a patient with an autoimmune disorder) for 24 hours at 50% effective concentration (EC50) of less than about nM, less than about 0.8 nM, less than about 0.6 nM, less than about 0.4 nM, less than about 0.3 nM or less than about 0.25 nM, less than about 0.2 nM, less than about 0.1 nM, less than about 0.05 nM, less than about 0.02 nM, less than about 0.01 nM, or less than about 0.005 nM. In preferred embodiments, the multispecific (e.g. bispecific) antibodies of the invention achieve lysis of 50% of plasmablasts when incubated with isolated PBMC (e.g. isolated PBMC from a patient with an autoimmune disorder) for 24 hours at a concentration of about 1 nM or less, about 0.8 nM or less, about 0.6 nM or less, about 0.4 nM or less, about 0.3 nM or less, about 0.25 nM or less, about 0.02 nM or less, about 0.01 nM or less, about 0.007 nM or less, or about 0.005 nM or less.

Plasmablast depletion is measured by FACS whereby plasmablasts are identified as CD19(+) CD20(−) CD27(+) cells. In some embodiments, the multispecific (e.g. bispecific) antibodies of the invention may achieve lysis of plasmablasts when incubated with isolated PBMC (e.g. isolated PBMC from a patient with an autoimmune disorder) for 24 hours at a 90% effective concentration (EC90) of less than about 1 nM, less than about 0.8 nM, less than about 0.6 nM, less than about 0.4 nM, less than about 0.3 nM, less than about 0.2 nM, less than about 0.1 nM, less than about 0.08 nM, or less than about 0.06 nM. In preferred embodiments, the multispecific (e.g. bispecific) antibodies of the invention achieve lysis of 90% of plasmablasts when incubated with isolated PBMC (e.g. isolated PBMC from a patient with an autoimmune disorder) for 24 hours at a concentration of less than about 1 nM, less than about 0.8 nM, less than about 0.6 nM, less than about 0.4 nM, less than about 0.3 nM, less than about 0.2 nM, less than about 0.1 nM, less than about 0.08 nM, or less than about 0.06 nM. Plasmablast depletion is measured by FACS whereby plasmablasts are identified as CD19(+) CD20(−) CD27(+) cells.

In some embodiments, the multispecific (e.g. bispecific) antibodies of the invention may achieve lysis of plasmablasts when incubated with isolated PBMC (e.g. isolated PBMC from a patient with an autoimmune disorder) for 24 hours at a 99% effective concentration (EC99) of less than about 1 nM, less than about 0.9 nM, less than about 0.8 nM, less than about 0.7 nM, or less than about 0.6 nM. In some embodiments, the multispecific (e.g. bispecific) antibodies of the invention achieve lysis of 99% of plasmablasts when incubated with isolated PBMC (e.g. isolated PBMC from a patient with an autoimmune disorder) for 24 hours at a concentration of less than about 1 nM, less than about 0.9 nM, less than about 0.8 nM, less than about 0.7 nM, or less than about 0.6 nM. Plasmablast depletion is measured by FACS whereby plasmablasts are identified as CD19(+) CD20(−) CD27(+) cells.

Cytokine release syndrome (CRS) represents one of the most frequent serious adverse effects of T cell-engaging immunotherapies for the treatment of cancers such as multiple myeloma (Shimabukuro-Vornhagen, A. et. al (2018) Cytokine release syndrome. J Immunother Cancer, 6(1) 56). Without being bound by theory, CRS may result from a large and/or rapid secretion of cytokines, for example because of activation and/or proliferation of immune effector cells. Elevated cytokine levels, such as IFNγ, IL-1β, IL-6, IL-2, IL-10, and/or granzyme B, are observed following treatment of multiple myeloma with T cell-engaging immunotherapies.

As autoimmune disorders are associated with high cytokine levels even prior to treatment (see Kunz, M. and Ibrahim, S. (2009) Cytokines and Cytokine Profiles in Human Autoimmune Diseases and Animal Models of Autoimmunity. Mediators of Inflammation, Article ID 979258; and Andreakos et. al (2002), Cytokines and anti-cytokine biologicals in autoimmunity: present and future. Cytokine & Growth Factor Reviews, Issues 4-5, Pages 299-313), T cell-engaging immunotherapies might be considered an unattractive therapy for autoimmune disorders. However, the present inventors have surprisingly identified that the multispecific (e.g. bispecific) antibodies of the invention may be administered at a therapeutically effective dose against the autoimmune disorders of the invention with no significant T cell activation or minimal T cell activation and with no significant cytokine production or minimal cytokine production. Accordingly, the multispecific (e.g. bispecific) antibodies of the invention can treat or manage the autoimmune disorders of the invention, with minimal risk of adverse events associated with T cell activation and/or cytokine production e.g. CRS.

In one aspect, the present invention provides a method for treatment or management of an autoimmune disorder with a multispecific (e.g. bispecific) antibody that binds to an antigen that promotes activation of one or more T cells (e.g. CD3) and BCMA, wherein the treatment comprises the administration of the multispecific (e.g. bispecific) antibody at a therapeutically effective dose with no significant T cell activation or minimal T cell activation.

As used herein, “no significant T cell activation or minimal T cell activation” refers to less than 20%, less than 15%, less than 10%, or less than 5% of T cells activated above the baseline of isolated PBMC or whole blood (e.g. isolated PBMC or whole blood from a patient with an autoimmune disorder), as measured by surface expression of the activation maker CD69.

Preferably, “no significant T cell activation or minimal T cell activation” refers to less than 20% of T cells activated above the baseline of isolated PBMC or whole blood (e.g. isolated PBMC or whole blood from a patient with an autoimmune disorder), as measured by surface expression of the activation maker CD69. Preferably, T-cell activation is reported for CD3+ T cells (e.g. CD3+CD4+ T cells or CD3+CD8+ T cells, or both).

As used herein, the “baseline” of T cells activated (e.g. the “baseline” of CD8(+) T cells expressing CD69) is defined as the percentage of relevant T cells (e.g. CD8(+) T cells) expressing the relevant activation marker (e.g. CD69) in a control sample of the isolated PBMC or whole blood (e.g. the isolated PBMC or whole blood from a patient with an autoimmune disorder). Preferably, the control sample is treated with a control anti-CD3 antibody e.g. a control 2+1 anti-HEL anti-CD3 antibody. Preferably, T-cell activation is reported for CD3+ T cells (e.g. CD3+CD4+ T cells or CD3+CD8+ T cells, or both). In some embodiments, there is no significant T cell activation or minimal T cell activation when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. T-cell activation can be measured by staining for T cell lineage (CD3, CD4 and CD8) and activation markers (CD69, CD25, and CD154). Preferably, T-cell activation is reported for CD3+ T cells (either CD3+CD4+ T cells or CD3+CD8+ T cells, or both).

In preferred embodiments, there is no significant increase in CD8(+) T cells expressing CD69 or minimal increase in CD8(+) T cells expressing CD69 when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. In preferred embodiments, there is less than 10%, less than 5%, less than 4%, less than about 3%, less than 2%, or less than 1% of CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. In some embodiments, there is no increase in frequency of CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours.

In some embodiments, there is less than 10%, less than 5%, less than 4%, less than about 3%, less than 2%, or less than 1% of CD4(+) T cells expressing CD69 and CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. In some embodiments, there is no increase in CD4(+)T cells expressing CD69 and CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours.

In some embodiments, there is no significant T cell activation or minimal T cell activation when the multispecific (e.g. bispecific) antibodies of the invention are incubated with isolated PBMC or whole blood (e.g. isolated PBMC or whole blood from a patient with an autoimmune disorder) at the 90% effective concentration (EC90) for plasmablast lysis for 24 hours. T-cell activation can be measured by staining for T cell lineage (CD3, CD4 and CD8) and activation markers (CD69, CD25, and CD154). Preferably, T-cell activation is reported for CD3+ T cells (either CD3+CD4+ T cells or CD3+CD8+ T cells, or both). In preferred embodiments, there is no significant increase in CD8(+) T cells expressing CD69 or minimal increase in CD8(+) T cells expressing CD69 when the multispecific (e.g. bispecific) antibodies of the invention are incubated with isolated PBMC or whole blood (e.g. isolated PBMC or whole blood from a patient with an autoimmune disorder) at the 90% effective concentration (EC90) for plasmablast lysis for 24 hours. In preferred embodiments, there is less than 20%, less than 15%, less than 10%, less than 5%, less than 4%, less than about 3%, less than 2%, or less than 1% of CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 90% effective concentration (EC90) for plasmablast lysis for 24 hours.

In some embodiments, there is less than 20%, less than 15%, less than 10%, less than 5%, less than 4%, less than about 3%, less than 2%, or less than 1% of CD4(+) T cells expressing CD69 and CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 90% effective concentration (EC90) for plasmablast lysis for 24 hours. In some embodiments, there is no increase in CD4(+) T cells expressing CD69 and CD8(+) T cells expressing CD69 above the baseline when the multispecific (e.g. bispecific) antibodies of the invention are incubated with isolated PBMC or whole blood (e.g. isolated PBMC or whole blood from a patient with an autoimmune disorder) at the 90% effective concentration (EC90) for plasmablast lysis for 24 hours.

In some embodiments, there is less than 40%, less than 30%, less than 20%, less than 15%, less than 10%, or less than 5% of CD8(+) T cells expressing CD69 above the baseline when the multispecific (e.g. bispecific) antibodies of the invention are incubated with isolated PBMC or whole blood (e.g. isolated PBMC or whole blood PBMC from a patient with an autoimmune disorder) at the 99% effective concentration (EC99) for plasmablast lysis for 24 hours. Preferably, T-cell activation is reported for CD3+ T cells (either CD3+CD4+ T cells or CD3+CD8+ T cells, or both).

In some embodiments, the multispecific (e.g. bispecific) antibodies of the invention may achieve lysis of more than 40%, more than 45%, more than 50%, more than 55%, more than 60%, more than 65%, more than 70%, more than 75%, more than 80%, more than 85%, more than 90%, more than 95% or about 100% of plasmablasts when incubated with isolated PBMC or whole blood (e.g. isolated PBMC or whole blood from a patient with an autoimmune disorder) for 24 hours, at a concentration wherein the number of CD8(+) T cells expressing CD69 is increased by less than about 20%, less than about 15%, less than about 10%, less than about 5%, less than about 4%, less than about 3%, less than about 2%, or less than about 1% above the baseline.

In some embodiments, the multispecific (e.g. bispecific) antibodies of the invention may achieve lysis of about 50%, about 90%, or about 99% of plasmablasts when incubated with isolated PBMC or whole blood (e.g. isolated PBMC or whole blood from a patient with an autoimmune disorder) for 24 hours, at a concentration wherein the number of CD8(+) T cells expressing CD69 is less than about 40%, less about than 30%, less than about 20%, less than about 15%, less than about 10%, less than about 5%, less than about 4%, less than about 3%, less than about 2%, or less than about 1% above the baseline. In one aspect, the present invention provides a method for treatment or management of an autoimmune disorder with a multispecific (e.g. bispecific) antibody that binds to an antigen that promotes activation of one or more T cells (e.g. CD3) and BCMA, wherein the treatment comprises the administration of the multispecific (e.g. bispecific) antibody at a therapeutically effective dose with no significant cytokine production or minimal cytokine production.

As used herein, “no significant cytokine production or minimal cytokine production” refers to cytokine levels less than 20 pg/mL, less than 10 pg/mL or less than 5 pg/mL above the baseline of isolated PBMC or whole blood (e.g. isolated PBMC or whole blood from a patient with an autoimmune disorder). Preferably, “no significant cytokine production or minimal cytokine production” refers to cytokine levels less than 5 pg/mL above the baseline cytokine levels in the donor sample. Cytokine levels may be measured using the MSD Pro-inflammatory I assay.

As used herein, the “baseline” cytokine level (e.g. the “baseline” level of IFNγ) is defined as the level of relevant cytokine (e.g. IFNγ) in a control sample of the isolated PBMC or whole blood (e.g. the isolated PBMC or whole blood from a patient with an autoimmune disorder). Preferably, the control sample is treated with a control anti-CD3 antibody e.g. a control 2+1 anti-HEL anti-CD3 antibody. Cytokine levels may be measured using the MSD Pro-inflammatory I assay.

In some embodiments, there is no significant cytokine production or minimal cytokine production when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. Production of the cytokines: IFNγ, IL-6, IL-2, IL-10, IL-1β, granzyme B and/or perforin is measured using the MSD Pro-inflammatory I assay. In some embodiments, the level of pro-inflammatory cytokines (e.g. IFNγ, IL-6, IL-2, IL-10, IL-1β, granzyme B and/or perforin) is less than 50 pg/mL, less than 20 pg/mL, less than 10 pg/mL or less than 5 pg/mL above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours.

In preferred embodiments, there is no significant increase in IFNγ or minimal increase in IFNγ when the multispecific (e.g. bispecific) antibodies of the invention can be incubated with isolated PBMC or whole blood (e.g. isolated PBMC or whole blood from a patient with an autoimmune disorder) at the 50% effective concentration (EC50) for plasmablast lysis for 24 hours. In some embodiments, the level of IFNγ is less than 50 pg/mL, less than 20 pg/mL, less than 10 pg/mL or less than 5 pg/mL above the baseline when the multispecific (e.g. bispecific) antibodies of the invention are incubated with isolated PBMC or whole blood (e.g. isolated PBMC or whole blood from a patient with an autoimmune disorder) at the 50% effective concentration (EC50) for plasmablast lysis for 24 hours. In some embodiments, the multispecific (e.g. bispecific) antibodies of the invention may achieve lysis of more than 40%, more than 45%, more than 50%, more than 55%, more than 60%, more than 65%, more than 70%, more than 75%, more than 80%, more than 85%, more than 90%, more than 95% or about 100% of plasmablasts when incubated with isolated PBMC or whole blood (e.g. isolated PBMC or whole blood from a patient with an autoimmune disorder) for 24 hours, at a concentration wherein IFNγ is increased by less than about 50 pg/ml, less than about 10 pg/ml, or less than about 5 pg/ml above the baseline.

Anti-IL-6 receptor therapy is used to treat CRS given the central role of IL-6 in driving CRS (Shimabukuro-Vornhagen, A. et. al (2018) Cytokine release syndrome. J Immunother Cancer, 6(1) 56). In some embodiments, the level of IL-6 is, less than 10 pg/mL or less than 5 pg/mL above the baseline when the multispecific (e.g. bispecific) antibody of the invention is incubated with isolated PBMC or whole blood (e.g. isolated PBMC or whole blood from a patient with an autoimmune disorder) at the 50% effective concentration (EC50) for plasmablast lysis for 24 hours.

In one aspect, the present invention provides a method for treatment or management of an autoimmune disorder with a multispecific (e.g. bispecific) antibody that binds to an antigen that promotes activation of one or more T cells (e.g. CD3) and BCMA, wherein the treatment comprises the administration of the multispecific (e.g. bispecific) antibody at a therapeutically effective dose with minimal CRS.

As used herein, “minimal CRS” refers to a lowered incidence of CTCAE v. 5.0 Grade 1 cytokine release syndrome (CRS) in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment. Preferably, “minimal CRS” refers to CTCAE v. 5.0 Grade 1 CRS. CTCAE v. 5.0 Grade 1 CRS is defined by the NIH, National Cancer Institute, Division of Cancer Treatment and Diagnosis (DCTD), Cancer Therapy Evaluation Program (CTEP).

In some embodiments, the method for treatment or management of the present invention results in a lowered incidence of cytokine release syndrome in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

In some embodiments, the method for treatment or management of the present invention results in a lowered incidence of infection in the patient by at least 5%, 10%, 15%, 20%, 25%, 30%, 35%, 40%, 45%, 50%, 55%, 60%, 65%, 70%, 75%, 80%, 85%, 90%, at least 95%, or 100% relative to a reference treatment.

The Multispecific Antibody

The multispecific (e.g. bispecific) antibodies of the invention specifically bind to BCMA and to an antigen that promotes activation of one or more T cells (e.g. CD3). The terms “antibody against BCMA and an antigen that promotes activation of one or more T cells (e.g. CD3)”, or “an antibody that binds to BCMA and to an antigen that promotes activation of one or more T cells (e.g. CD3),” refer to a multispecific (e.g. bispecific) antibody that is capable of binding to BCMA and an antigen that promotes activation of one or more T cells (e.g. CD3) with sufficient affinity such that the antibody is useful as a therapeutic agent. This is achieved by making a molecule which comprises a first antibody, or antigen-binding fragment, that binds to BCMA and a second antibody, or antigen-binding fragment, that binds to an antigen that promotes activation of one or more T cells (e.g. CD3). Such multispecific antibodies may be trispecific antibodies or bispecific antibodies. In preferred embodiments, the multispecific antibodies are bispecific antibodies.

The term “BCMA” as used herein relates to human B cell maturation antigen, also known as BCMA; TR17_HUMAN, TNFRSF17 (UniProt Q02223), which is a member of the tumor necrosis receptor superfamily that is preferentially expressed in differentiated plasma cells. The extracellular domain of BCMA consists according to UniProt of amino acids 1-54 (or 5-51). The terms “antibody against BCMA”, “anti-BCMA antibody” or “an antibody that binds to BCMA” as used herein relate to an antibody specifically binding to the extracellular domain of BCMA.

The term “specifically binding to BCMA” refers to an antibody that is capable of binding to the defined target with sufficient affinity such that the antibody is useful as a therapeutic agent in targeting BCMA. In some embodiments, an antibody specifically binding to BCMA does not bind to other antigens, or does not bind to other antigens with sufficient affinity to produce a physiological effect.

In some embodiments, the extent of binding of an anti-BCMA antibody to an unrelated, non-BCMA protein is about 10-fold preferably >100-fold less than the binding of the antibody to BCMA as measured, e.g., by surface plasmon resonance (SPR) e.g. Biacore®, enzyme-linked immunosorbent (ELISA) or flow cytometry (FACS). In one embodiment the antibody that binds to BCMA has a dissociation constant (Kd) of 10⁻⁸ M or less, preferably from 10⁻⁸ M to 10⁻³ M, preferably from 10⁻⁹ M to 10⁻¹³ M.

In one embodiment the anti-BCMA antibody binds to an epitope of BCMA that is conserved among BCMA from different species, preferably among human and cynomolgus, and in addition preferably also to mouse and rat BCMA.

Preferably the anti-BCMA antibody specifically binds to a group of BCMA, consisting of human BCMA and BCMA of non-human mammalian origin, preferably BCMA from cynomolgus, mouse and/or rat. Anti-BCMA antibodies are analyzed by ELISA for binding to human BCMA using plate-bound BCMA. For this assay, an amount of plate-bound BCMA preferably 1.5 μg/mL and concentration(s) ranging from 0.1 pM to 200 nM of anti-BCMA antibody are used.

The multispecific (e.g. bispecific) antibodies of the invention bind to an antigen that promotes activation of one or more T cells, e.g. a T cell antigen. In preferred embodiments, the T cell antigen is a human T cell antigen. The antigen that promotes activation of one or more T cells may be selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In preferred embodiments, the antigen that promotes activation of one or more T cells is CD3, e.g. human CD3. Accordingly, in preferred embodiments, multispecific (e.g. bispecific) antibodies of the invention bind to CD3, e.g. human CD3.

The term “CD3” refers to the human CD3 protein multi-subunit complex. The CD3 protein multi-subunit complex is composed to 6 distinctive polypeptide chains. Thus the term includes a CD3γ chain (SwissProt P09693), a CD3δ chain (SwissProt P04234), two CD3ε chains (SwissProt P07766), and one CD3ζ chain homodimer (SwissProt 20963), and which is associated with the T cell receptor α and β chain. The term encompasses “full-length,” unprocessed CD3, as well as any CD3 variant, isoform and species homolog which is naturally expressed by cells (including T cells) or can be expressed on cells transfected with genes or cDNA encoding those polypeptides.

The term “specifically binding to CD3” refers to an antibody that is capable of binding to the defined target with sufficient affinity such that the antibody is useful as a therapeutic agent in targeting CD3. In some embodiments, an antibody specifically binding to CD3 does not bind to other antigens, or does not bind to other antigens with sufficient affinity to produce a physiological effect.

The multispecific (e.g. bispecific) antibodies of the invention can be analyzed by SPR, e.g. Biacore®, for binding to CD3. In some embodiments, the bispecific antibodies bind to human CD3 with a dissociation constant (K_{D}) of about 10⁻⁷ M or less, a K_{D} of about 10⁻⁸ M or less, a K_{D} of about 10⁻⁹ M or less, a K_{D} of about 10⁻¹⁰ M or less, a K_{D} of about 10⁻¹¹ M or less, or a K_{D} of about 10⁻¹² M or less, as determined by a surface plasmon resonance assay, preferably measured using Biacore 8K at 25° C. In preferred embodiments, the multispecific (e.g. bispecific) antibodies bind to human CD3 with a dissociation constant (K_{D}) of about 10⁻⁸ M or less.

The term “antibody” herein encompasses various antibody structures, including but not limited to monoclonal antibodies, polyclonal antibodies, multispecific (e.g. bispecific) antibodies and antibody fragments so long as they exhibit the desired antigen-binding activity.

A “heavy chain” comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM).

A “light chain” comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

The term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen. Thus, the term refers to a molecule other than an intact antibody that comprises a portion of an intact antibody that binds the antigen to which the intact antibody binds.

Examples of antibody fragments include but are not limited to Fv, Fab, Fab′, Fab′-SH, F(ab′)2; diabodies; linear antibodies; single-chain antibody molecules (e.g. scFv); and multispecific (e.g. bispecific) antibodies formed from antibody fragments.

The terms “Fab fragment” and “Fab” are used interchangeably herein and contain a single light chain (i.e. a constant domain CL and a VL) and a single heavy chain (i.e. the constant domain CH1 and a VH). The heavy chain of a Fab fragment is not capable of forming a disulfide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulfide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein consisting of two single-chain variable fragments (scFvs) on a single peptide chain. One of the scFvs binds to T cells via the CD3 receptor, and the other to a tumor cell antigen.

A “diabody” is a small bivalent and bispecific antibody fragment comprising a heavy (VH) chain variable domain connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

A “DARPin” is a bispecific ankyrin repeat molecule. DARPins are derived from natural ankyrin proteins, which can be found in the human genome and are one of the most abundant types of binding proteins. A DARPin library module is defined by natural ankyrin repeat protein sequences, using 229 ankyrin repeats for the initial design and another 2200 for subsequent refinement. The modules serve as building blocks for the DARPin libraries. The library modules resemble human genome sequences. A DARPin is composed of 4 to 6 modules. Because each module is approx. 3.5 kDa, the size of an average DARPin is 16-21 kDa. Selection of binders is done by ribosome display, which is completely cell-free and is described in He M. and Taussig M J., Biochem Soc Trans. 2007, November; 35(Pt 5):962-5.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, M D (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)).

TABLE 1
CDR definitions

| | Kabat | Chothia | IMGT |
|---|---|---|---|
| VH CDR1 | 31-35 | 26-32 | 27-38 |
| VH CDR2 | 50-65 | 52-56 | 56-65 |
| VH CDR3 | 95-102 | 95-102 | 105-117 |
| VL CDR1 | 24-34 | 24-34 | 27-38 |
| VL CDR2 | 50-56 | 50-56 | 56-65 |
| VL CDR3 | 89-97 | 89-97 | 105-117 |

For heavy chain constant region amino acid positions discussed in the invention, numbering is according to the EU index first described in Edelman, G. M, et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human IgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat set forth in Kabat et al. (supra.).

The antibodies of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies.

The term “humanized antibody” refers to antibodies in which the framework or “complementarity determining regions” (CDRs) have been modified to comprise the CDR of an immunoglobulin of different specificity as compared to that of the parent immunoglobulin. For example, a murine CDR may be grafted into the framework region of a human antibody to prepare the “humanized antibody.” See, e.g., Riechmann, L., et al., Nature 332 (1988) 323-327; and Neuberger, M. S., et al., Nature 314 (1985) 268-270. In some embodiments, “humanized antibodies” are those in which the constant region has been additionally modified or changed from that of the original antibody to generate the properties of the antibodies according to the invention, especially in regard to Clq binding and/or Fc receptor (FcR) binding.

The term “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties of the antibodies according to the invention, especially in regard to Clq binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S. L., et al., Proc. Natl. Acad. Sci. USA 81 (1984) 6851-6855; U.S. Pat. Nos. 5,202,238 and 5,204,244.

The terms “Fc region” and “Fc” are used interchangeably herein and refer to the portion of a native immunoglobulin that is formed by two Fc chains. Each “Fc chain” comprises a constant domain CH2 and a constant domain CH3. Each Fc chain may also comprise a hinge region. A native Fc region is homodimeric. In some embodiments, the Fc region may contain modifications to enforce Fc heterodimerization.

The term “Fc part” refers to the portion of an antibody of the invention, or antigen binding fragment thereof, which corresponds to the Fc region.

There are five major classes of heavy chain constant region, classified as IgA, IgG, IgD, IgE and IgM, each with characteristic effector functions designated by isotype. For example, IgG is separated into four subclasses known as IgG1, IgG2, IgG3, and IgG4. Ig molecules interact with multiple classes of cellular receptors. For example, IgG molecules interact with three classes of Fcγ receptors (FcγR) specific for the IgG class of antibody, namely FcγRI, FcγRII, and FcγRIII. The important sequences for the binding of IgG to the FcγR receptors have been reported to be located in the CH2 and CH3 domains.

The antibodies of the invention or antigen-binding fragments thereof may be any isotype, i.e. IgA, IgD, IgE, IgG and IgM, and synthetic multimers of the four-chain immunoglobulin (Ig) structure. In preferred embodiments, the antibodies or antigen-binding fragments thereof are IgG isotype. The antibodies or antigen-binding fragments can be any IgG subclass, for example IgG1, IgG2, IgG3, or IgG4 isotype. In preferred embodiments, the antibodies or antigen-binding fragments thereof are of an IgG1 isotype.

In some embodiments, the antibodies comprise a heavy chain constant region that is of IgG isotype. In some embodiments, the antibodies comprise a portion of a heavy chain constant region that is of IgG isotype. In some embodiments, the IgG constant region or portion thereof is an IgG1, IgG2, IgG3, or IgG4 constant region. Preferably, the IgG constant region or portion thereof is an IgG1 constant region.

The antibodies of the invention or antigen-binding fragments thereof may comprise a lambda light chain or a kappa light chain.

In preferred embodiments, the antibodies or antigen-binding fragments thereof comprise a light chain that is a kappa light chain. In some embodiments, the antibody or antigen-binding fragment comprises a light chain comprising a light chain constant region (CL) that is a kappa constant region.

In some embodiments, the antibody comprises a light chain comprising a light chain variable region (VL) that is a kappa variable region. Preferably, the kappa light chain comprises a VL that is a kappa VL and a CL that is a kappa CL.

Alternatively, the antibodies or antigen-binding fragments thereof may comprise a light chain that is a lambda light chain. In some embodiments, the antibody or antigen-binding fragment comprises a light chain comprising a light chain constant region (CL) that is a lambda constant region. In some embodiments, the antibody comprises a light chain comprising a light chain variable region (VL) that is a lambda variable region.

Engineered antibodies and antigen-binding fragments thereof include those in which modifications have been made to framework residues within the VH and/or VL. Such modifications may improve the properties of the antibody, for example to decrease the immunogenicity of the antibody and/or improve antibody production and purification.

Antibodies and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The antibodies of the invention and antigen-binding fragments thereof also include derivatives that are modified (e.g., by the covalent attachment of any type of molecule to the antibody) such that covalent attachment does not prevent the antibody from binding to its epitope, or otherwise impair the biological activity of the antibody. Examples of suitable derivatives include, but are not limited to fucosylated antibodies, glycosylated antibodies, acetylated antibodies, PEGylated antibodies, phosphorylated antibodies, and amidated antibodies.

Minor variations in the amino acid sequences of antibodies of the invention are contemplated as being encompassed by the present invention, providing that the variations in the amino acid sequence(s) maintain at least 75%, more preferably at least 80%, at least 90%, at least 95%, and most preferably at least 99% sequence identity to the antibody of the invention or antigen-binding fragment thereof as defined anywhere herein.

Antibodies of the invention may include variants in which amino acid residues from one species are substituted for the corresponding residue in another species, either at the conserved or non-conserved positions. In one embodiment, amino acid residues at non-conserved positions are substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Antibody Format

Formats for multispecific (e.g. bispecific) antibodies are known in the state of the art. For example, bispecific antibody formats are described in Kontermann R E, mAbs 4:2 1-16 (2012); Holliger P., Hudson P J, Nature Biotech. 23 (2005) 1126-1136, Chan A C, Carter P J Nature Reviews Immunology 10, 301-316 (2010) and Cuesta A M et al., Trends Biotech 28 (2011) 355-362.

The multispecific (e.g. bispecific) antibodies of the invention may have any format. Multispecific (e.g. bispecific) antibody formats include, for example, multivalent single chain antibodies, diabodies and triabodies, and antibodies having the constant domain structure of full length antibodies to which further antigen-binding domains (e.g., single chain Fv, a tandem scFv, a VH domain and/or a VL domain, Fab, or (Fab)₂) are linked via one or more peptide-linkers, as well as antibody mimetics such as DARPins. In some embodiments, the multispecific (e.g. bispecific) antibodies of the invention have the format of an scFv such as a bispecific T cell engager (BITE®).

In some embodiments, the antibodies of the invention are single chain antibodies which comprise a first domain which binds to BCMA, a second domain which binds to a T cell antigen (e.g. CD3), and a third domain which comprises two polypeptide monomers, each comprising a hinge, a CH2 domain and a CH3 domain, wherein the two polypeptide monomers are fused to each other via a peptide linker (e.g. (hinge-CH2-CH3-linker-hinge-CH2-CH3).

The “valency” of an antibody denotes the number of binding domains. As such, the terms “bivalent”, “trivalent”, and “multivalent” denote the presence of two binding domains, three binding domains, and multiple binding domains, respectively. The multispecific (e.g. bispecific) antibodies of the invention may have more than one binding domain capable of binding to each target antigen (i.e., the antibody is trivalent or multivalent). In preferred embodiments, the multispecific (e.g. bispecific) antibodies of the invention have more than one binding domain capable of binding to the same epitope of each target antigen. In some embodiments, the multispecific (e.g. bispecific) antibodies of the invention have more than one binding domain capable of binding to different epitopes on each target antigen.

The multispecific (e.g. bispecific) antibodies of the invention may be bivalent, trivalent or tetravalent. In preferred embodiments, the multispecific (e.g. bispecific) antibody is trivalent, preferably wherein the trivalent antibody is bivalent for BCMA. Thus, the bispecific antibody may be trivalent, wherein the trivalent antibody is bivalent for BCMA.

The multispecific (e.g. bispecific) antibodies can be full length from a single species, or can be chimerized or humanized. For an antibody with more than two antigen-binding domains, some binding domains may be identical, as long as the protein has binding domains for two different antigens.

The multispecific (e.g. bispecific) antibodies of the invention can have a bispecific heterodimeric format. In some embodiments, the bispecific antibody comprises two different heavy chains and two different light chains. In other embodiments, the multispecific (e.g. bispecific) antibody comprises two identical light chains and two different heavy chains. In some embodiments, in the multispecific (e.g. bispecific) antibodies of the invention one of the two pairs of heavy chain and light chain (HC/LC) specifically binds to CD3 and the other one specifically binds to BCMA.

In embodiments in which the bispecific antibodies of the invention are bivalent, they may comprise one anti-BCMA antibody and one anti-CD3 antibody (referred to herein as the “1+1” format).

In embodiments in which the BCMA and CD3 antibodies are Fabs, the bivalent bispecific antibodies in the 1+1 format may have the format: CD3 Fab-BCMA Fab (i.e. when no Fc is present). Alternatively, the bispecific antibodies may have the format: Fc-CD3 Fab-BCMA Fab; Fc-BCMA Fab-CD3 Fab; or BCMA Fab-Fc-CD3 Fab (i.e. when an Fc is present). In preferred embodiments, the bivalent bispecific antibodies have the format BCMA Fab-Fc-CD3 Fab.

“CD3 Fab-BCMA Fab” means that the CD3 Fab is bound via its N-terminus to the C-terminus of the BCMA Fab.

“Fc-BCMA Fab-CD3 Fab” means that the BCMA Fab is bound via its C-terminus to the N-terminus of the Fc, and the CD3 Fab is bound via its C-terminus to the N-terminus of the BCMA Fab.

“Fc-CD3 Fab-BCMA Fab” means that the CD3 Fab is bound via its C-terminus to the N-terminus of the Fc, and the BCMA Fab is bound via its C-terminus to the N-terminus of the CD3 Fab.

“BCMA Fab-Fc-CD3 Fab” means that the BCMA and CD3 Fab fragments are bound via their C-terminus to the N-terminus of the Fc.

In embodiments in which the bispecific antibodies of the invention are trivalent, they may comprise two anti-BCMA antibodies and one anti-CD3 antibody (referred to herein as the “2+1” format).

In embodiments in which the BCMA and CD3 antibodies are Fabs, the trivalent bispecific antibodies in the 2+1 format may have the format: CD3 Fab-BCMA Fab-BCMA Fab; or BCMA Fab-CD3 Fab-BCMA Fab (i.e. when no Fc is present). Alternatively, the bispecific antibodies may have the format: BCMA Fab-Fc-CD3 Fab-BCMA Fab; BCMA Fab-Fc-BCMA Fab-CD3 Fab; or CD3 Fab-Fc-BCMA Fab-BCMA Fab (i.e. when an Fc is present). In preferred embodiments, the trivalent bispecific antibodies have the format BCMA Fab-Fc-CD3 Fab-BCMA Fab.

“CD3 Fab-BCMA Fab-BCMA Fab” means that the CD3 Fab is bound via its C-terminus to the N-terminus of the first BCMA Fab, and the first BCMA Fab is bound via its C-terminus to the N-terminus of the second BCMA Fab.

“BCMA Fab-CD3 Fab-BCMA Fab” means that the first BCMA Fab is bound via its C-terminus to the N-terminus of the CD3 Fab, and the CD3 Fab is bound via its C-terminus to the N-terminus of the second BCMA Fab.

“BCMA Fab-Fc-CD3 Fab-BCMA Fab” means that the first BCMA Fab and the CD3 Fab are bound via their C-terminus to the N-terminus of the Fc, and the second BCMA Fab is bound via its C-terminus to the N-terminus of the CD3 Fab.

“BCMA Fab-Fc-BCMA Fab-CD3 Fab” means that the first BCMA Fab and the second BCMA Fab are bound via their C-terminus to the N-terminus of the Fc, and the CD3 Fab is bound via its C-terminus to the N-terminus of the second BCMA Fab.

“CD3 Fab-Fc-BCMA Fab-BCMA Fab” means that the CD3 Fab and the first BCMA Fab are bound via their C-terminus to the N-terminus of the Fc, and the second BCMA Fab is bound via its C-terminus to the N-terminus of the first BCMA Fab.

In some embodiments, the bispecific antibodies of the invention may comprise not more than one BCMA Fab specifically binding to BCMA, and not more than one CD3 Fab specifically binding to CD3 and not more than one Fc part.

In some embodiments, the bispecific antibody comprises not more than one CD3 Fab specifically binding to CD3, not more than two BCMA Fabs specifically binding to BCMA and not more than one Fc part. In some embodiments, not more than one CD3 Fab and not more than one BCMA Fab are linked to the Fc part and linking is performed via C-terminal binding of the Fab(s) to the hinge region of the Fc part. In some embodiments, the second BCMA Fab is linked via its C-terminus either to the N-terminus of the CD3 Fab or to the hinge region of the Fc part and is therefore between the Fc part of the bispecific antibody and the CD3 Fab.

In embodiments comprising two BCMA Fabs, the BCMA Fabs are preferably derived from the same antibody and are preferably identical in the CDR sequences, variable domain sequences VH and VL and/or the constant domain sequences CH1 and CL. Preferably, the amino acid sequences of the two BCMA Fab are identical.

The bispecific antibodies of the invention can also comprise scFvs instead of the Fabs. Thus, in some embodiments, the bispecific antibodies have any one of the above formats, wherein each Fab is replaced with a corresponding scFv.

The components, e.g. the Fab fragments, of the bispecific antibodies of the invention may be chemically linked together by the use of an appropriate linker according to the state of the art. In preferred embodiments, a (Gly4-Ser1)₃ linker is used (Desplancq D K et al., Protein Eng. 1994 August; 7(8):1027-33 and Mack M. et al., PNAS Jul. 18, 1995 vol. 92 no. 15 7021-7025). “Chemically linked” (or “linked”) as used herein means that the components are linked by covalent binding. As the linker is a peptidic linker, such covalent binding is usually performed by biochemical recombinant means. For example, the binding may be performed using a nucleic acid encoding the VL and/or VH domains of the respective Fab fragments, the linker and the Fc part chain if the antibody comprises an Fc.

In the event that a linker is used, this linker may be of a length and sequence sufficient to ensure that each of the first and second domains can, independently from each other, retain their differential binding specificities.

Antibody Sequences

In some embodiments, the multispecific (e.g. bispecific) antibody comprises an anti-BCMA antibody, or antigen binding fragment thereof, comprising a CDR1H, CDR2H, CDR3H, CDR1L, CDR2L, and CDR3L region combination selected from the group of:
a) CDR1H region of SEQ ID NO:21, CDR2H region of SEQ ID NO:22, CDR3H region of SEQ ID NO:17, CDR1L region of SEQ ID NO:23, CDR2L region of SEQ ID NO:24, and CDR3L region of SEQ ID NO:20;
b) CDR1H region of SEQ ID NO:21, CDR2H region of SEQ ID NO:22, CDR3H region of SEQ ID NO:17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO:17, CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO:17, CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO:17, CDR1L region of SEQ ID NO:31, CDR2L region of SEQ ID NO:32, and CDR3L region of SEQ ID NO:33; and
g) CDR1H region of SEQ ID NO:15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20.

In any of the embodiments disclosed herein, a CDR1L region of SEQ ID NO:18 may be replaced with a CDR1L region of SEQ ID NO:67, and a CDR2L region of SEQ ID NO:19 may be replaced with a CDR2L region of SEQ ID NO:68. Accordingly, in some embodiments the multispecific (e.g. bispecific) antibody may comprise an anti-BCMA antibody, or antigen binding fragment thereof, comprising CDR1H region of SEQ ID NO:15, CDR2H region of SEQ ID NO:16, CDR3H region of SEQ ID NO:17, CDR1L region of SEQ ID NO:67, CDR2L region of SEQ ID NO:68, and CDR3L region of SEQ ID NO:20.

In any of the embodiments disclosed herein, a CDR1L region of SEQ ID NO:27 may be replaced with a CDR1L region of SEQ ID NO:71; and a CDR2L region of SEQ ID NO:28 may be replaced with a CDR2L region of SEQ ID NO:72. Accordingly, in some embodiments the multispecific (e.g. bispecific) antibody may comprise an anti-BCMA antibody, or antigen binding fragment thereof, comprising CDR1H region of SEQ ID NO:21, CDR2H region of SEQ ID NO:22, CDR3H region of SEQ ID NO:17, CDR1L region of SEQ ID NO:71, CDR2L region of SEQ ID NO:72, and CDR3L region of SEQ ID NO:20;

In any of the embodiments disclosed herein, a CDR1L region of SEQ ID NO:25 may be replaced with a CDR1L region of SEQ ID NO:69; and a CDR2L region of SEQ ID NO:26 may be replaced with a CDR2L region of SEQ ID NO:70. Accordingly, in some embodiments the multispecific (e.g. bispecific) antibody may comprise an anti-BCMA antibody, or antigen binding fragment thereof, comprising CDR1H region of SEQ ID NO:21, CDR2H region of SEQ ID NO:22, CDR3H region of SEQ ID NO:17, CDR1L region of SEQ ID NO:69, CDR2L region of SEQ ID NO:70, and CDR3L region of SEQ ID NO:20.

In preferred embodiments, the multispecific (e.g. bispecific) antibody comprises an anti-BCMA antibody, or antigen binding fragment thereof, comprises an anti-BCMA antibody, or antigen binding fragment thereof, comprising a CDR1H, CDR2H, CDR3H CDR1L, CDR2L and CDR3L region combination selected from:
a) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and CDR3L region of SEQ ID NO:20;
b) CDR1H region of SEQ ID NO:21, CDR2H region of SEQ ID NO:22, CDR3H region of SEQ ID NO:17, CDR1L region of SEQ ID NO:25, CDR2L region of SEQ ID NO:26, and CDR3L region of SEQ ID NO:20; or
c) CDR1H region of SEQ ID NO:15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20.

In particularly preferred embodiments, the multispecific (e.g. bispecific) antibody comprises an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO:17 and a VL region comprising a CDR1L region of SEQ ID NO:27, a CDR2L region of SEQ ID NO:28 and a CDR3L region of SEQ ID NO:20. In some embodiments, the multispecific (e.g. bispecific) antibody comprises an anti-BCMA antibody, or antigen binding fragment thereof, comprising a VH and a VL selected from the group consisting of:
a) a VH region of SEQ ID NO:10 and a VL region of SEQ ID NO:12,
b) a VH region of SEQ ID NO:10 and a VL region of SEQ ID NO:13,
c) a VH region of SEQ ID NO:10 and a VL region of SEQ ID NO:14,
d) a VH region of SEQ ID NO:38 and a VL region of SEQ ID NO:12,
e) a VH region of SEQ ID NO:39 and a VL region of SEQ ID NO:12,
f) a VH region of SEQ ID NO:40 and a VL region of SEQ ID NO:12, or
g) a VH region of SEQ ID NO:9 and a VL region of SEQ ID NO:11.

In some embodiments, the multispecific (e.g. bispecific) antibody comprises an anti-BCMA antibody, or antigen binding fragment thereof, comprising a VH and a VL selected from the group consisting of:
a) a VH comprising an amino acid sequence that is at least 75% identical, at least 90% identical, at least 95% identical, or identical to the amino acid sequence of SEQ ID NO:10 and a VL comprising an amino acid sequence that is at least 90% identical, at least 95% identical, or identical to the amino acid sequence of SEQ ID NO:14;
b) a VH comprising an amino acid sequence that is at least 75% identical, at least 90% identical, at least 95% identical, or identical to the amino acid sequence of SEQ ID NO:10 and a VL comprising an amino acid sequence that is at least 75% identical, at least 90% identical, at least 95% identical, or identical to the amino acid sequence of SEQ ID NO:13; or
c) a VH comprising an amino acid sequence that is at least 75% identical, at least 90% identical, at least 95% identical to, or identical to the amino acid sequence of SEQ ID NO:9 and a VL comprising an amino acid sequence that is at least 75% identical, at least 90% identical, at least 95% identical, or identical to the amino acid sequence of SEQ ID NO:11.

In some embodiments, the multispecific (e.g. bispecific) antibody comprises an anti-BCMA antibody, or antigen binding fragment thereof, comprising a VH and a VL selected from the group consisting of:
a) a VH region of SEQ ID NO:10 and a VL region of SEQ ID NO:13,
b) a VH region of SEQ ID NO:10 and a VL region of SEQ ID NO:14, or
c) a VH region of SEQ ID NO:9 and a VL region of SEQ ID NO:11.

In particularly preferred embodiments, the anti-BCMA antibody, or antigen binding fragment thereof, comprises a VH region of SEQ ID NO:10 and a VL region of SEQ ID NO: 14.

In some embodiments, the multispecific (e.g. bispecific) antibody comprises an anti-CD3 antibody, or antigen binding fragment thereof.

Examples of anti-CD3 antibodies include OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT371(Ala-Ala) (Teplizumab) and NI-0401 (Foralumab).

The first anti-CD3 antibody generated was OKT3 (muromonab-CD3), a murine antibody binding to the CD3; domain. Subsequent anti-CD3 antibodies include humanized or human antibodies, and engineered antibodies, for example antibodies comprising modified Fc regions.

Anti-CD3 antibodies may recognise an epitope on a single polypeptide chain, for example APA 1/1 or SP34 (Yang S J, The Journal of Immunology (1986) 137; 1097-1100), or a conformational epitope located on two or more subunits of CD3, for example WT31, 7D6, UCHT-1 (see WO2000041474) and Leu-4. Clinical trials have been carried out using several anti-CD3 antibodies, including BC-3 (Anasetti et al., Transplantation 54: 844 (1992) and H2C (WO2008119567A2). Anti-CD3 antibodies in clinical development include HuM291 (visilizumab) (Norman et al., Transplantation. 2000 Dec. 27; 70(12):1707-12.) Hu291 (PDL), ChAglyCD3 (Otelixizumab) (H Waldmann), hOKT371(Ala-Ala) (Teplizumab) (J Bluestone and Johnson and Johnson) and (NI-0401) Foralumab.

Any anti-CD3 antibody or antigen-binding fragment thereof may be suitable for use in the multispecific (e.g. bispecific) antibodies of the present invention. For example, the multispecific (e.g. bispecific) antibodies may comprise an anti-CD3 antibody selected from OKT3, TR66, APA 1/1, SP34, CH2527, WT31, 7D6, UCHT-1, Leu-4, BC-3, H2C, HuM291 (visilizumab), Hu291 (PDL), ChAglyCD3 (Otelixizumab), hOKT371(Ala-Ala) (Teplizumab) and NI-0401 (Foralumab).

In some embodiments, the multispecific (e.g. bispecific) antibody of the invention comprises a humanized SP34 antibody or antigen-binding fragment thereof.

In some preferred embodiments, the anti-CD3 antibody, or antigen binding fragment thereof, may be derived from SP34 and may have similar sequences and the same properties with regard to epitope binding as antibody SP34.

In some embodiments, the multispecific (e.g. bispecific) antibody comprises an anti-CD3 antibody, or antigen binding fragment thereof, comprising a variable domain VH comprising the heavy chain CDRs of SEQ ID NO: 1, 2 and 3 as respectively heavy chain CDR1H, CDR2H and CDR3H and a variable domain VL comprising the light chain CDRs of SEQ ID NO: 4, 5 and 6 as respectively light chain CDR1L, CDR2L and CDR3L.

In some embodiments, the multispecific (e.g. bispecific) antibody comprises an anti-CD3 antibody, or antigen binding fragment thereof, comprising the variable domains of SEQ ID NO:7 (VH) and SEQ ID NO:8 (VL). In some embodiments, the multispecific (e.g. bispecific) antibody comprises an anti-CD3 antibody, or antigen binding fragment thereof, comprising a variable region VH comprising an amino acid sequence that is at least 75% identical, at least 90% identical, at least 95% identical or identical to the amino acid sequence of SEQ ID NO:7 and a variable region VL comprising an amino acid sequence that is at least 75% identical, at least 90% identical, at least 95% identical, or identical to the amino acid sequence of SEQ ID NO:8.

In some embodiments, the multispecific (e.g. bispecific) antibody comprises an anti-BCMA antibody, or antigen binding fragment thereof, comprising a CDR1H, CDR2H, CDR3H, CDR1L, CDR2L, and CDR3L region combination selected from the group of:
a) CDR1H region of SEQ ID NO:21, CDR2H region of SEQ ID NO:22, CDR3H region of SEQ ID NO:17, CDR1L region of SEQ ID NO:23, CDR2L region of SEQ ID NO:24, and CDR3L region of SEQ ID NO:20;
b) CDR1H region of SEQ ID NO:21. CDR2H region of SEQ ID NO:22, CDR3H region of SEQ ID NO:17, CDR1L region of SEQ ID NO:25, CDR2L region of SEQ ID NO:26, and CDR3L region of SEQ ID NO:20;
c) CDR1H region of SEQ ID NO:21, CDR2H region of SEQ ID NO:22, CDR3H region of SEQ ID NO:17 CDR1L region of SEQ ID NO:27, CDR2L region of SEQ ID NO:28, and CDR3L region of SEQ ID NO:20;
d) CDR1H region of SEQ ID NO:29, CDR2H region of SEQ ID NO:30, CDR3H region of SEQ ID NO:17 CDR1L region of SEQ ID NO:31, CDR2L region of SEQ ID NO:32, and CDR3L region of SEQ ID NO:33;
e) CDR1H region of SEQ ID NO:34, CDR2H region of SEQ ID NO:35, CDR3H region of SEQ ID NO:17 CDR1L region of SEQ ID NO:31, CDR2L region of SEQ ID NO:32, and CDR3L region of SEQ ID NO:33;
f) CDR1H region of SEQ ID NO:36, CDR2H region of SEQ ID NO:37, CDR3H region of SEQ ID NO:17 CDR1L region of SEQ ID NO:31, CDR2L region of SEQ ID NO:32, and CDR3L region of SEQ ID NO:33; or
g) CDR1H region of SEQ ID NO:15, CDR2H region of SEQ ID NO:16, CDR3H region of SEQ ID NO:17, CDR1L region of SEQ ID NO:18, CDR2L region of SEQ ID NO:19, and CDR3L region of SEQ ID NO:20, and an anti-CD3 antibody, or antigen binding fragment thereof, comprising a CDR1H region of SEQ ID NO:1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In particularly preferred embodiments, the multispecific (e.g. bispecific) antibody comprises: an anti-BCMA antibody, or antigen binding fragment thereof, comprising a VH region comprising a CDR1H region of SEQ ID NO:21, a CDR2H region of SEQ ID NO:22 and a CDR3H region of SEQ ID NO:17 and a VL region comprising a CDR1L region of SEQ ID NO:27, a CDR2L region of SEQ ID NO:28 and a CDR3L region of SEQ ID NO:20; and an anti-CD3 antibody, or antigen binding fragment thereof, comprising a CDR1H region of SEQ ID NO:1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In some embodiments, the multispecific (e.g. bispecific) antibody comprises an anti-BCMA antibody, or antigen binding fragment thereof, comprising a VH and a VL selected from the group consisting of:
a) a VH region of SEQ ID NO:10 and a VL region of SEQ ID NO:12;
b) a VH region of SEQ ID NO:10 and a VL region of SEQ ID NO:13;
c) a VH region of SEQ ID NO:10 and a VL region of SEQ ID NO:14;
d) a VH region of SEQ ID NO:38 and a VL region of SEQ ID NO:12;
e) a VH region of SEQ ID NO:39 and a VL region of SEQ ID NO:12;
f) a VH region of SEQ ID NO:40 and a VL region of SEQ ID NO:12; or
g) a VH region of SEQ ID NO:9 and a VL region of SEQ ID NO:11, and
   an anti-CD3 antibody, or antigen binding fragment thereof, comprising a VH region of SEQ ID NO:7 and a VL region of SEQ ID NO:8.

In particularly preferred embodiments, the multispecific (e.g. bispecific) antibody comprises an anti-BCMA antibody, or antigen binding fragment thereof, comprising a VH region of SEQ ID NO:10 and a VL region of SEQ ID NO: 14, and an anti-CD3 antibody, or antigen binding fragment thereof, comprising a VH region of SEQ ID NO:7 and a VL region of SEQ ID NO:8.

In some embodiments, the multispecific (e.g. bispecific) antibody comprises an anti-BCMA antibody, or antigen binding fragment thereof, comprising the CDR3H, CDR3L, CDR1H, CDR2H, CDR1L, and CDR2L of one of GSK2857916, AMG-420, AMG-701, JNJ-957, JNJ-64007957, PF-06863135, REGN-5458, or TNB-383B. In some embodiments, the multispecific (e.g. bispecific) antibody comprises an anti-BCMA antibody, or antigen binding fragment thereof, comprising the VH and VL of one of GSK2857916, AMG-420, AMG-701, JNJ-957, JNJ-64007957, PF-06863135, REGN-5458, or TNB-383B.

Fc

The multispecific (e.g. bispecific) antibodies of the invention may have an Fc or may not have an Fc. In preferred embodiments, the multispecific (e.g. bispecific) antibodies of the invention comprise an Fc, preferably a human Fc.

In certain embodiments, the Fc is a variant Fc, e.g., an Fc sequence that has been modified (for example by amino acid substitution, deletion and/or insertion) relative to a parent Fc sequence (for example an unmodified Fc polypeptide that is subsequently modified to generate a variant), to provide desirable structural features and/or biological activity,

Accordingly, the multispecific (e.g. bispecific) antibodies of the invention may comprise an Fc comprising one or more modifications, typically to alter one or more functional properties of the antibody, such as serum half-life, complement fixation, Fc receptor binding, and/or antigen-dependent cellular cytotoxicity. The Fc may be linked to the anti-BCMA and/or anti-CD3 Fab fragments in the antibodies of the invention.

The presence of an Fc has the advantage of extending the elimination half-life of the antibody. The multispecific (e.g. bispecific) antibodies of the invention may have an elimination half-life in mice or cynomolgus monkeys, preferably cynomolgus monkeys, of longer than 12 hours, preferably 3 days or longer. In some embodiments, the multispecific (e.g. bispecific) antibodies of the invention have an elimination half-life of about 1 to 12 days, which allows at least once or twice/week administration.

Reduced Effector Function

Preferably, the multispecific (e.g. bispecific) antibodies of the invention comprise an Fc region (e.g. of IgG1 subclass) that comprises modifications to avoid FcR and Clq binding and minimize ADCC/CDC. This provides the advantage that the bispecific antibody mediates its tumour cell killing efficacy purely by the powerful mechanism of effector cell, e.g. T cell, redirection/activation. Therefore, additional mechanisms of action, such as effects on the complement system and on effector cells expressing FcR, are avoided and the risk of side-effects, such as infusion-related reactions, is decreased.

In preferred embodiments, the multispecific (e.g. bispecific) antibodies of the invention comprise an IgG, particularly IgG1, Fc region comprising the modifications L234A, L235A and P329G (numbered according to EU numbering).

Heterodimerization

The multispecific (e.g. bispecific) antibodies of the invention may be heteromultimeric antibodies. Such heteromultimeric antibodies may comprise modifications in regions involved in interactions between antibody chains to promote correct assembly of the antibodies.

For example, the multispecific (e.g. bispecific) antibodies of the invention may comprise an Fc having one or more modification(s) in the CH2 and CH3 domain to enforce Fc heterodimerization.

Alternatively or in addition, the multispecific (e.g. bispecific) antibodies of the invention may comprise modifications in the CH1 and CL region to promote preferential pairing between the heavy chain and light chain of a Fab fragment.

A number of strategies exist for promoting heterodimerization. These strategies may include the introduction of asymmetric complementary modifications into each of two antibody chains, such that both chains are compatible with each other and thus able to form a heterodimer, but each chain is not able to dimerize with itself. Such modifications may encompass insertions, deletions, conservative and non-conservative substitutions and rearrangements.

Heterodimerization may be promoted by the introduction of charged residues to create favorable electrostatic interactions between a first antibody chain and a second antibody chain. For example, one or more positively charged amino acids amino acid may be introduced into a first antibody chain, and one or more negatively charged amino acids may be introduced into a corresponding positions in a second antibody chain

Alternatively or in addition, heterodimerization may be promoted by the introduction of steric hindrance between contacting residues. For example, one or more residues with a bulky side chain may be introduced into a first antibody chain, and a one or more residues able to accommodate the bulky side chain may be introduced into the second antibody chain.

Alternatively or in addition, heterodimerization may be promoted by the introduction of one or more modification(s) to the hydrophilic and hydrophobic residues at the interface between chains, in order make heterodimer formation more entropically and enthalpically favorable than homodimer formation.

A further strategy for promoting heterodimerization is to rearrange portions of the antibody chains such that each chain remains compatible only with a chain comprising corresponding rearrangements. For example, CrossMAb technology is based on the crossover of antibody domains in order to enable correct chain association. There are three main CrossMAb formats, these are: (i) CrossMAb^{Fab} in which the VH and VL are exchanged and the CH1 and CL are exchanged; (ii) CrossMAb^{VH-VL} in which the VH and VL are exchanged; and (iii) CrossMAb^{CH1-CL} in which the CH1 and CL are exchanged (Klein et al., 2016. MABS, 8(6):1010-1020).

In some embodiments, the multispecific (e.g. bispecific) antibodies of the invention may comprise an exchange of the VH and VL. In some embodiments, the multispecific (e.g. bispecific) antibodies of the invention may comprise an exchange of the CH1 and CL. In some embodiments, the multispecific (e.g. bispecific) antibodies of the invention may comprise an exchange of the VH and VL and an exchange of the CH1 and CL.

In preferred embodiments, the multispecific (e.g. bispecific) antibodies of the invention comprise an exchange of the VH and VL.

Other approaches to promoting heterodimerization include the use of a strand exchange engineered domain (SEED) (Davis et al., 2010. Protein Eng Des Sel, 23 (4); 195-202).

A combination of the above strategies may be used to maximise the efficiency of assembly while minimising the impact on antibody stability.

Fc Heterodimerization

In some embodiments, multispecific (e.g. bispecific) antibodies of the invention may have a heterodimeric Fc, for example they may comprise one heavy chain originating from an anti-BCMA antibody, and one heavy chain originating from an anti-CD3 antibody.

The multispecific (e.g. bispecific) antibodies of the invention may comprise a heterodimeric Fc which comprises one or more modification(s) which promotes the association of the first CH2 and/or CH3 domain with the second CH2 and/or CH3 domain. In preferred embodiments, the one or more modification(s) promote the association of the first CH3 domain with the second CH3 domain, for example by resulting in asymmetric modifications to the CH3 domain. The one or more modification(s) may comprise modifications selected from amino acid insertions, deletions, conservative and non-conservative substitutions and rearrangements, and combinations thereof.

Typically the first CH3 domain and the second CH3 domain are both engineered in a complementary manner so that each CH3 domain (or the heavy chain comprising it) can no longer homodimerize with itself but is forced to heterodimerize with the complementary engineered other CH3 domain (so that the first and second CH3 domain heterodimerize and no homodimers between the two first or the two second CH3 domains are formed).

The multispecific (e.g. bispecific) antibodies of the invention may comprise an Fc having one or more of “knob-into-holes” modification(s), which are described in detail with several examples in e.g. WO 96/027011, Ridgway, J. B., et al., Protein Eng. 9 (1996) 617-621, Merchant, A. M. et al., Nat. Biotechnol. 16 (1998) 677-68, and WO 98/050431.

In this method, the interaction surfaces of the two CH3 domains are altered to increase the heterodimerization of both Fc chains containing these two CH3 domains. One of the two CH3 domains (of the two Fc chains) can be the “knob”, while the other is the “hole”.

Accordingly, the multispecific (e.g. bispecific) antibodies of the invention may comprise two CH3 domains, wherein the first CH3 domain of the first Fc chain and the second CH3 domain of the second Fc chain each meet at an interface which comprises an original interface between the antibody CH3 domains, wherein said interface is altered to promote the formation of the antibody.

In some embodiments:
(i) the CH3 domain of one Fc chain is altered, so that within the original interface of the CH3 domain of the one Fc chain that meets the original interface of the CH3 domain of the other Fc chain, an amino acid residue is replaced with an amino acid residue having a larger side chain volume, thereby generating a protuberance within the interface of the CH3 domain of one Fc chain which is positionable in a cavity within the interface of the CH3 domain of the other Fc chain; and
ii) the CH3 domain of the other Fc chain is altered, so that within the original interface of the CH3 domain of the other Fc chain that meets the original interface of the CH3 domain of the one Fc chain, an amino acid residue is replaced with an amino acid residue having a smaller side chain volume, thereby generating a cavity within the interface of the CH3 domain of the other Fc chain within which a protuberance within the interface of the CH3 domain of the one Fc chain is positionable.

Preferably, said amino acid residue having a larger side chain volume is selected from the group consisting of arginine (R), phenylalanine (F), tyrosine (Y), tryptophan (W).

In some embodiments, the multispecific (e.g. bispecific) antibodies of the invention comprise a first CH3 domain comprising modification(s) at positions T366, L368 and Y407, e.g. T366S, L368A, and Y407V (numbered according to EU numbering).

In some embodiments, the multispecific (e.g. bispecific) antibodies of the invention comprise a second CH3 domain comprising a modification at position T366 (“knob modification”), e.g. T366W (numbered according to EU numbering).

In particularly preferred embodiments, the multispecific (e.g. bispecific) antibodies of the invention comprise a first CH3 domain comprising the modifications T366S, L368A, and Y407V, or conservative substitutions thereof, and a second CH3 domain comprising the modification T366W, or a conservative substitution thereof (numbered according to EU numbering).

In one embodiment, the multispecific (e.g. bispecific) antibodies of the invention comprise a first CH3 domain comprising the modification set forth in Table 2 and a second CH3 domain comprising the modifications set forth in Table 2.

TABLE 2
“Knob-into-holes” modification

| First CH3 domain | | Second CH3 domain | |
|---|---|---|---|
| KABAT | EU NUMBERING | KABAT | EU NUMBERING |
| T389S | T366S | | |
| L391A | L368A | T389W | T366W |
| Y438V | Y407V | | |

The multispecific (e.g. bispecific) antibodies of the invention may comprise one or more of the modification(s) set forth in U.S. Pat. Nos. 9,562,109 and 9,574,010 (incorporated herein by reference).

In some embodiments, the multispecific (e.g. bispecific) antibodies of the invention comprise a first CH3 domain comprising one or more modification(s) at positions T350, L351, F405 and/or Y407 (numbered according to EU numbering), e.g. T350V, L351Y, F405A and/or Y407V. In some embodiments, the multispecific (e.g. bispecific) antibodies of the invention comprise a first CH3 domain comprising modification(s) at positions T350, L351, F405 and Y407 (numbered according to EU numbering), e.g. T350V, L351Y, F405A and Y407V.

In some embodiments, the multispecific (e.g. bispecific) antibodies of the invention comprise a second CH3 domain comprising one or more modification(s) at positions T350, T366, K392 and/or T394 (numbered according to EU numbering), e.g. T350V, T366L, K392L and/or T394W. In some embodiments, the multispecific (e.g. bispecific) antibodies of the invention comprise a second CH3 domain comprising modification(s) at positions T350, T366, K392 and T394 (numbered according to EU numbering), e.g. T350V, T366L, K392L and T394W.

In preferred embodiments, the multispecific (e.g. bispecific) antibodies of the invention comprise a first CH3 domain comprising one or more modification(s) at positions T350, L351, F405 and/or Y407 (e.g. T350V, L351Y, F405A and/or Y407V) and a second CH3 domain comprising one or more modification(s) at positions T350, T366, K392 and/or T394 (e.g. T350V, T366L, K392L and/or T394W) (numbered according to EU numbering).

In particularly preferred embodiments, the multispecific (e.g. bispecific) antibodies of the invention comprise a first CH3 domain comprising modification(s) at positions T350, L351, F405 and Y407 (e.g. T350V, L351Y, F405A and Y407V) and a second CH3 domain comprising modification(s) at positions T350, T366, K392 and T394 (e.g. T350V, T366L, K392L and T394W) (numbered according to EU numbering).

The one or more modification(s) may modify electrostatic charges, hydrophobic/hydrophilic interactions, and/or steric interference between side chains.

In particularly preferred embodiments, the multispecific (e.g. bispecific) antibodies of the invention comprise a first CH3 domain comprising the modifications T350V, L351Y, F405A and Y407V, or conservative substitutions thereof, and a second CH3 domain comprising the modifications T350V, T366L, K392L and T394W, or conservative substitutions thereof (numbered according to EU numbering).

In one embodiment, the multispecific (e.g. bispecific) antibodies of the invention comprise a first CH3 domain comprising the modifications set forth in Table 3 and a second CH3 domain comprising the modifications set forth in Table 3.

TABLE 3
Fc Heterodimerization modifications

| First CH3 domain | | Second CH3 domain | |
|---|---|---|---|
| KABAT | EU NUMBERING | KABAT | EU NUMBERING |
| T371V | T350V | T371V | T350V |
| L372Y | L351Y | T389L | T366L |
| F436A | F405A | K420L | K392L |
| Y438V | Y407V | T422W | T394W |

Other techniques for CH3 modifications to enforce heterodimerization are contemplated as alternatives of the invention and are described e.g. in WO96/27011, WO98/050431, EP1870459, WO2007/110205, WO2007/147901, WO2009/089004, WO2010/129304, WO2011/90754, WO2011/143545, WO2012/058768, WO2013/157954, WO2013/157953, and WO2013/096291.

In some embodiments, the bispecific antibody according to the invention is of IgG2 isotype and the heterodimerization approach described in WO2010/129304 can be used.

Other Fc Modifications

In some embodiments, the bispecific antibodies of the invention may comprise an Fc, wherein both CH3 domains are altered by the introduction of cysteine (C) as the amino acid in the corresponding positions of each CH3 domain such that a disulphide bridge between both CH3 domains can be formed. The cysteines may be introduced at position 349 in one of the CH3 domains and at position 354 in the other CH3 domain (numbered according to EU numbering).

Preferably, the cysteine introduced at position 354 is in the first CH3 domain and the cysteine introduced at position 349 is in the second CH3 domain (numbered according to EU numbering).

The Fc may comprise modifications, such as D356E, L358M, N384S, K392N, V397M, and V422I (numbered according to EU numbering). Preferably, both CH3 domains comprise D356E and L358M (numbered according to EU numbering).

Light and Heavy Chain Heterodimerization

In the multispecific (e.g. bispecific) antibodies of the invention, one or more of the immunoglobulin heavy chains and light chains may comprise one or more modification(s), e.g. amino acid modifications that are capable of promoting preferential pairing of a specific heavy chain with a specific light chain when heavy chains and light chains are co-expressed or co-produced. Such modifications can provide considerably improved production/purification without changing biological properties such as binding to BCMA. In particular, by introduction of one or more modification(s) such as amino acid exchanges, light chain mispairing and the formation of side products in production can be significantly reduced and therefore yield is increased and purification is facilitated.

The one or more modification(s) may promote preferential heterodimer pairing by introducing steric hindrance, substitutions of charged amino acids with opposite charges and/or by hydrophobic or hydrophilic interactions. In preferred embodiments, the one or more modification(s) promote preferential heterodimer pairing by introducing steric hindrance and substitution(s) of charged amino acids with opposite charges.

The amino acid exchanges may be substitutions of charged amino acids with opposite charges (for example in the CH1/CL interface) which reduce light chain mispairing, e.g. Bence-Jones type side products.

In preferred embodiments, the one or more modification(s) assist light and heavy chain heterodimerization are amino acid modifications in the light and heavy chains outside of the CDRs.

The one or more modification(s) may be present in the anti-BCMA antibody or antigen-binding fragment thereof. Alternatively, the one or more modification(s) may be present in the anti-CD3 antibody or antigen-binding fragment thereof. In preferred embodiments, the one or more modification(s) are present in the anti-BCMA antibody or antigen-binding fragment thereof.

In some embodiments, the multispecific (e.g. bispecific) antibodies of the invention comprise an immunoglobulin heavy chain comprising a CH1 domain having amino acid modifications K147E/D and K213E/D (numbered according to EU numbering) and a corresponding immunoglobulin light chain comprising a CL domain having amino acid modifications E123K/R/H and Q124K/R/H (numbered according to Kabat). Preferably, the CH1 domain comprises the amino acid modifications K147E and K213E (numbered according to EU numbering) or conservative substitutions thereof, and the corresponding CL domain comprises the amino acid modifications E123R and Q124K or conservative substitutions thereof (numbered according to Kabat). Such multispecific (e.g. bispecific) antibodies can be produced in high yield and can be easily purified.

In one embodiment, the amino acid modifications described in Table 4 can be in the BCMA antibody or in the CD3 antibody.

In one embodiment, the bispecific antibodies of the invention are bivalent, and comprise one anti-BCMA antibody or antigen-binding fragment thereof and one anti-CD3 antibody or antigen-binding fragment thereof (the “1+1” format), wherein:
(a) the BCMA antibody or antigen-binding fragment thereof (e.g. BCMA Fab) comprises a CH1 domain having amino acid modifications set forth in Table 4 and a corresponding CL domain having the amino acid modifications Table 4; or
(b) the CD3 antibody or antigen-binding fragment thereof (e.g. CD3 Fab) comprises a CH1 domain having amino acid modifications set forth in Table 4 and a corresponding CL domain having the amino acid modifications Table 4.

In one embodiment, the bispecific antibodies of the invention are trivalent and comprise two anti-BCMA antibodies or antigen-binding fragments thereof and one anti-CD3 antibody or antigen-binding fragment thereof (the “2+1” format), wherein:
(a) one or both BCMA antibodies or antigen-binding fragments thereof (e.g. BCMA Fabs) comprises a CH1 domain having amino acid modifications set forth in Table 4 and a corresponding CL domain having the amino acid modifications Table 4; or
(b) the CD3 antibody (e.g. CD3 Fab) comprises a CH1 domain having amino acid modifications set forth in Table 4 and a corresponding CL domain having the amino acid modifications Table 4.

In particular, each BCMA antibody (e.g. BCMA Fab) may comprise a CH1 domain having amino acid modifications set forth in Table 4 and a corresponding CL domain having the amino acid modifications

TABLE 4
Light and heavy chain heterodimerization modifications

| CH1 domain | | CL domain | |
|---|---|---|---|
| KABAT | EU NUMBERING | KABAT | EU NUMBERING |
| K145E | K147E | E123R | E123R |
| K221E | K213E | Q124K | Q124K |

In a preferred embodiment, the multispecific (e.g. bispecific) antibodies of the invention comprise the modifications set forth in Table 4 in combination with the modifications set forth in Table 2. Thus, in one embodiment, the bispecific antibodies of the invention are bivalent, and comprise:
(a) one anti-BCMA antibody or antigen-binding fragment thereof and one anti-CD3 antibody or antigen-binding fragment thereof (the “1+1” format), wherein (i) the BCMA antibody or antigen-binding fragment thereof (e.g. BCMA Fab) comprises a CH1 domain that comprises the amino acid modifications K147E and K213E, and a corresponding CL domain that comprises the amino acid modifications E123R and Q124K (i.e. the modifications set forth in Table 4), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 4); and
(b) a first CH3 domain comprising the modifications T366S, L368A, and Y407V, and a second CH3 domain comprising the modification T366W (i.e. the modifications set forth in Table 2).

In one embodiment, the bispecific antibodies of the invention are trivalent and comprise:
(a) two anti-BCMA antibodies or antigen-binding fragments thereof and one anti-CD3 antibody or antigen-binding fragment thereof (the “2+1” format), wherein (i) one or both BCMA antibodies or antigen-binding fragments thereof (e.g. BCMA Fabs) comprises a CH1 domain that comprises the amino acid modifications K147E and K213E, and a corresponding CL domain that comprises the amino acid modifications E123R and Q124K (i.e. the modifications set forth in Table 4), or (ii) the CD3 antibody or antigen-binding fragment thereof (e.g. CD3 Fab) comprises a CH1 domain that comprises the amino acid modifications K147E and K213E, and a corresponding CL domain that comprises the amino acid modifications E123R and Q124K (i.e. the modifications set forth in Table 4); and
(b) a first CH3 domain comprising the modifications T366S, L368A, and Y407V, and a second CH3 domain comprising the modification T366W (i.e. the modifications set forth in Table 2).

In particular, each BCMA antibody (e.g. BCMA Fab) may comprise a CH1 domain having amino acid modifications set forth in Table 4 and a corresponding CL domain having the amino acid modifications Table 4. In preferred embodiments, the first Fc chain is bound at the N-terminus of the Fc to the C-terminus of the first anti-BCMA antibody, and the second Fc chain is bound at the N-terminus of the Fc to the C-terminus of the anti-CD3 antibody.

In some embodiments, the multispecific (e.g. bispecific), antibodies of the invention comprise an immunoglobulin heavy chain comprising a CH1 domain having amino acid modifications at one or more of position(s) A141, L145, K147, Q175 (numbered according to EU numbering) and a corresponding immunoglobulin light chain comprising a CL domain having amino acid modifications at one or more of position(s) F116, Q124, L135, T178 (numbered according to Kabat). Preferably, the CH1 domain comprises the amino acid modifications A141W, L145E, K147T, Q175E or conservative substitutions thereof (numbered according to EU numbering), and the corresponding CL domain comprises the amino acid modifications F116A, Q124R, L135V, T178R or conservative substitutions thereof (numbered according to Kabat).

In one embodiment, the multispecific (e.g. bispecific) antibodies of the invention comprise a CH1 domain having amino acid modifications set forth in Table 5 and a corresponding immunoglobulin light chain comprising a CL domain having amino acid modifications set forth in Table 5. In embodiments where the multispecific (e.g. bispecific) antibodies of the invention comprise an anti-BCMA antibody, or antigen binding fragment thereof of the invention, and an anti-CD3 antibody, or antigen binding fragment thereof, of the invention, the amino acid modifications described in Table 5 can be in the BCMA antibody or in the CD3 antibody.

In one embodiment, the bispecific antibodies of the invention are bivalent, and comprise one anti-BCMA antibody and one anti-CD3 antibody (the “1+1” format), wherein:
(a) the BCMA antibody (e.g. BCMA Fab) comprises a CH1 domain having amino acid modifications set forth in Table 5 and a corresponding CL domain having the amino acid modifications Table 5; or
(b) the CD3 antibody (e.g. CD3 Fab) comprises a CH1 domain having amino acid modifications set forth in Table 5 and a corresponding CL domain having the amino acid modifications Table 5.

In one embodiment, the bispecific antibodies of the invention are trivalent and comprise two anti-BCMA antibodies and one anti-CD3 antibody (the “2+1” format), wherein:
(a) one or both BCMA antibodies (e.g. BCMA Fabs) comprises a CH1 domain having amino acid modifications set forth in Table 5 and a corresponding CL domain having the amino acid modifications Table 5; or
(b) the CD3 antibody (e.g. CD3 Fab) comprises a CH1 domain having amino acid modifications set forth in Table 5 and a corresponding CL domain having the amino acid modifications Table 5.

In particular preferred embodiments, each BCMA antibody (e.g. BCMA Fab) may comprise a CH1 domain having amino acid modifications set forth in Table 5 and a corresponding CL domain having the amino acid modifications Table 5.

TABLE 5
Light and heavy chain heterodimerization modifications

| CH1 domain | | CL domain | |
|---|---|---|---|
| KABAT | EU NUMBERING | KABAT | EU NUMBERING |
| A139W | A141W | F116A | F116A |
| L143E | L145E | Q124R | Q124R |
| K145T | K147T | L135V | L135V |
| Q179E | Q175E | T178R | T178R |

In a preferred embodiment, the multispecific (e.g. bispecific) antibodies of the invention comprise the amino acid modifications set forth in Table 5 in combination with the amino acid modifications set forth in Table 3. Thus, in one embodiment, the bispecific antibodies of the invention are bivalent, and comprise:
(a) one anti-BCMA antibody and one anti-CD3 antibody (the “1+1” format), wherein (i) the BCMA antibody (e.g. BCMA Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 5), or (ii) the CD3 antibody (e.g. CD3 Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 5); and
(b) a first CH3 domain comprising the modifications T350V, L351Y, F405A and Y407V, and a second CH3 domain comprising the modifications T350V, T366L, K392L and T394W (i.e. the modifications set forth in Table 3).

In preferred embodiments, the first Fc chain is bound at the N-terminus of the Fc to the C-terminus of the anti-BCMA antibody, and the second Fc chain is bound at the N-terminus of the Fc to the C-terminus of the anti-CD3 antibody.

In one embodiment, the bispecific antibodies of the invention are trivalent and comprise:
(a) two anti-BCMA antibodies and one anti-CD3 antibody (the “2+1” format), wherein (i) one or both BCMA antibodies (e.g. BCMA Fabs) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 5), or (ii) the CD3 antibody (e.g. CD3 Fab) comprises a CH1 domain that comprises the amino acid modifications A141W, L145E, K147T and Q175E, and a corresponding CL domain that comprises the amino acid modifications F116A, Q124R, L135V and T178R (i.e. the modifications set forth in Table 5); and
(b) a first CH3 domain comprising the modifications T350V, L351Y, F405A and Y407V, and a second CH3 domain comprising the modifications T350V, T366L, K392L and T394W (i.e. the modifications set forth in Table 3).

In particular, each BCMA antibody (e.g. BCMA Fab) comprises a CH1 domain having amino acid modifications set forth in Table 5 and a corresponding CL domain having the amino acid modifications Table 5. In preferred embodiments, the first Fc chain is bound at the N-terminus of the Fc to the C-terminus of the first anti-BCMA antibody, and the second Fc chain is bound at the N-terminus of the Fc to the C-terminus of the anti-CD3 antibody.

Alternatively, the CH1 domain may comprise an amino acid modification at position Q175 (numbered according to EU numbering) and the corresponding CL domain may comprise amino acid modifications at one or more of position(s) F116, Q124, L135, T178 (numbered according to Kabat). The CH1 domain may comprise the amino acid modification Q175K (numbered according to EU numbering), or a conservative substitution thereof, and the corresponding CL domain may comprise amino acid modifications F116A, Q124R, L135V, T178R (numbered according to Kabat), or conservative substitutions thereof.

In alternative embodiments, the CH1 domain may comprises an amino acid modification at position Q175 (numbered according to EU numbering) and the corresponding CL domain may comprises amino acid modifications at one or more of position(s) Q124, L135, Q160, T180 (numbered according to Kabat). The CH1 domain may comprise the amino acid modification Q175K (numbered according to EU numbering), or a conservative substitution thereof, and the corresponding CL domain may comprise the amino acid modifications Q124E, L135W, Q160E and T180E, or conservative substitutions thereof (numbered according to Kabat).

The multispecific (e.g. bispecific) antibodies of the invention may additionally comprise an amino acid substitution at position 49 of the VL region selected from the group of amino acids tyrosine (Y), glutamic acid (E), serine (S), and histidine (H) and/or an amino acid substitution at position 74 of the VL region that is threonine (T) or alanine (A).

CrossMAb

The multispecific (e.g. bispecific) antibodies of the invention may comprise CrossMAb technology. CrossMAb technology is based on the crossover of antibody domains in order to enable correct chain association. It is used to facilitate multispecific (e.g. bispecific) antibody formation.

There are three main CrossMAb formats, these are: (i) CrossMAb^{Fab} in which the VH and VL are exchanged and the CH1 and CL are exchanged; (ii) CrossMAb^{VH-VL}, in which the VH and VL are exchanged; and (iii) CrossMAb^{CH1-CL} in which the CH1 and CL are exchanged (Klein et al., 2016. MABS, 8(6):1010-1020).

CrossMAb technology is known in the state of the art. Bispecific antibodies wherein the variable domains VL and VH or the constant domains CL and CH1 are replaced by each other are described in WO2009080251 and WO2009080252.

In one or more of the antibodies or antigen-binding fragments within the multispecific (e.g. bispecific) antibodies of the invention, the variable domains VL and VH or the constant domains CL and CH1 may be replaced by each other. In some embodiments, the multispecific (e.g. bispecific) antibodies of the invention may comprise an exchange of the VH and VL and an exchange of the CH1 and CL. Thus, the multispecific (e.g. bispecific) antibodies of the invention may comprise a crossover light chain and a crossover heavy chain. As used herein, a “crossover light chain” is a light chain that may comprise a VH-CL, a VL-CH1 or a VH-CH1. A “crossover heavy chain” as used herein is a heavy chain that may comprise a VL-CH1, a VH-CL or a VL-CL.

In some embodiments, the multispecific (e.g. bispecific) antibodies of the invention comprise:
(a) a light chain and a heavy chain of an antibody specifically binding to CD3; and
(b) a light chain and heavy chain of an antibody specifically binding to BCMA,
wherein the variable domains VL and VH and/or the constant domains CL and CH1 are replaced by each other in (i) the anti-BCMA antibody; and/or (ii) the anti-CD3 antibody.

In some embodiments, the variable domains VL and VH or the constant domains CL and CH1 of the anti-CD3 antibody or antigen binding fragment thereof are replaced by each other. More preferably, the variable domains VL and VH of the anti-CD3 antibody or antigen binding fragment thereof are replaced by each other.

In embodiments in which the bispecific antibodies in the 1+1 format have the format: CD3 Fab-BCMA Fab (i.e. when no Fc is present); Fc-CD3 Fab-BCMA Fab; Fc-BCMA Fab-CD3 Fab; or BCMA Fab-Fc-CD3 Fab, the bispecific antibodies may comprise the CrossMAb format, e.g. CrossMAb^{Fab}, CrossMAb^{VH-VL} or CrossMAb^{CH1-CL}. The BCMA Fab may have the CrossMAb format, e.g. CrossMAb^{Fab}, CrossMAb^{VH-VL} or CrossMAb^{CH1-CL}. Alternatively, the CD3 Fab may have the CrossMAb format, e.g. CrossMAb^{Fab}, CrossMAb^{VH-VL} or CrossMAb^{CH1-CL}. In preferred embodiments, the CD3 Fab of the bispecific antibody comprises the CrossMAb^{VH-VL} format.

It is especially preferred for the bispecific antibodies of the invention having the 2+1 format to comprise CrossMAb technology. Thus, in embodiments in which the trivalent bispecific antibodies in the 2+1 format have the format: CD3 Fab-BCMA Fab-BCMA Fab; BCMA Fab-CD3 Fab-BCMA Fab (i.e. when no Fc is present); BCMA Fab-Fc-CD3 Fab-BCMA Fab; BCMA Fab-Fc-BCMA Fab-CD3 Fab; or CD3 Fab-Fc-BCMA Fab-BCMA Fab, the bispecific antibodies may comprise the CrossMAb format, e.g. CrossMAb^{Fab}, CrossMAb^{VH-VL} or CrossMAb^{CH1-CL}. The BCMA Fab may have the CrossMAb format, e.g. CrossMAb^{Fab}, CrossMAb^{VH-VL} or CrossMAb^{CH1-CL}. Alternatively, the CD3 Fab may have the CrossMAb format, e.g. CrossMAb^{Fab}, CrossMAb^{VH-VL} or CrossMAb^{CH1-CL}. In preferred embodiments, the CD3 Fab of the bispecific antibody comprises the CrossMAb^{VH-VL} format.

In some embodiments, the bispecific antibodies of the invention having the 1+1 format do not comprise CrossMAb technology, i.e. neither the anti-BCMA antibody nor the anti-CD3 antibody have the variable domains VL and VH or the constant domains CL and CH1 replaced by each other.

### Exemplary Embodiments

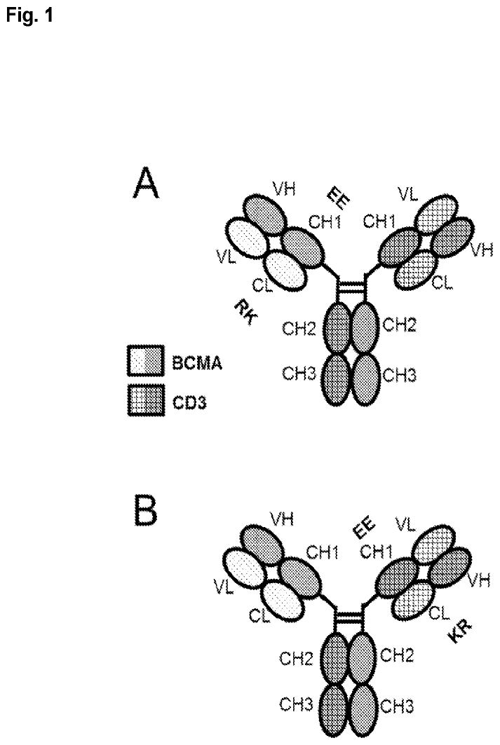

Exemplary embodiments are set out in FIGS. 1-3.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab-Fc-CD3 Fab. The anti-BCMA Fab fragment comprises the amino acid modifications set forth in Table 4 or Table 5. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 1A with the amino acid modifications set forth in Table 4.

In one embodiment, the bispecific antibodies according to the invention are bivalent bispecific antibodies comprising one Fab fragment of an anti-CD3 antibody, one Fab fragment of an anti-BCMA antibody and one Fc part according to the format BCMA Fab-Fc-CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 4 or Table 5. This embodiment is illustrated in FIG. 1B with the amino acid modifications set forth in Table 4.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab.

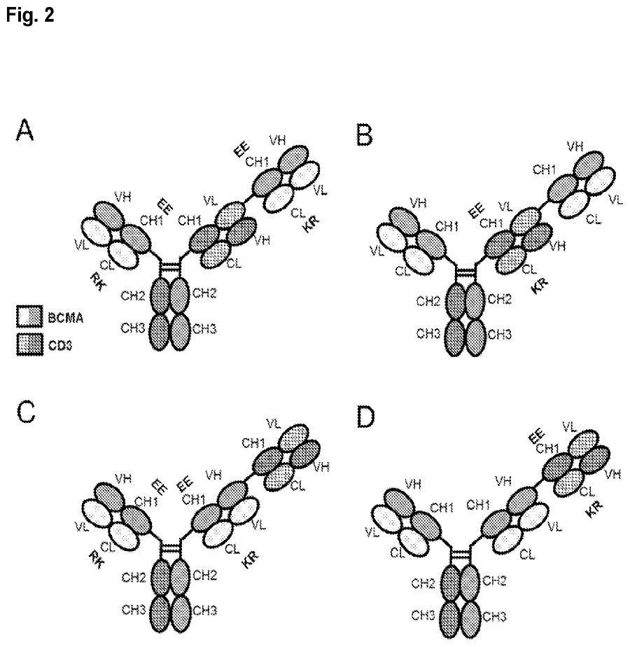

Each anti-BCMA Fab fragment comprises the amino acid modifications set forth in Table 4 or Table 5. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2A with the amino acid modifications set forth in Table 4.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab.

The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 4 or Table 5.

This embodiment is illustrated in FIG. 2B with the amino acid modifications set forth in Table 4.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-BCMA Fab-CD3 Fab.

Each anti-BCMA Fab fragment comprises the amino acid modifications set forth in Table 4 or Table 5. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 2C with the amino acid modifications set forth in Table 4.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-BCMA Fab-CD3 Fab.

The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 4 or Table 5. This embodiment is illustrated in FIG. 2D with the amino acid modifications set forth in Table 4.

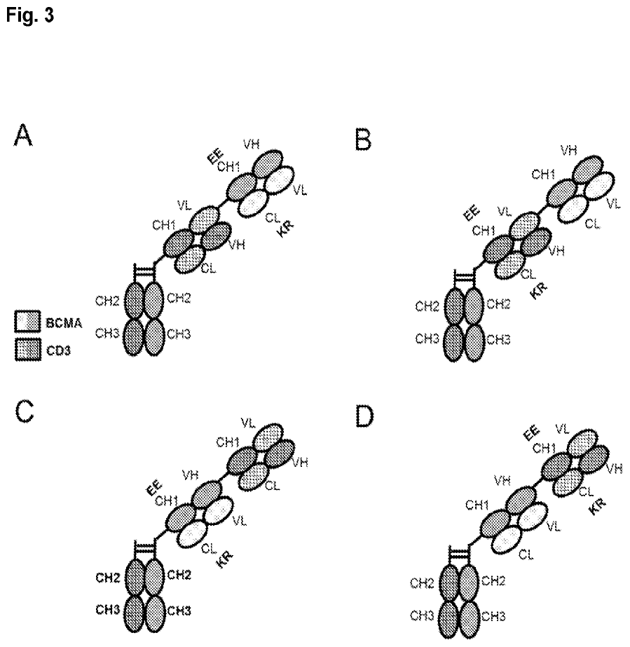

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-CD3 Fab-BCMA Fab. The anti-BCMA Fab fragment comprises the amino acid modifications set forth in Table 4 or Table 5. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 3A with the amino acid modifications set forth in Table 4.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-CD3 Fab-BCMA Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 4 or Table 5. This embodiment is illustrated in FIG. 3B with the amino acid modifications set forth in Table 4.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-BCMA Fab-CD3 Fab. The anti-BCMA Fab fragment comprises the amino acid modifications set forth in Table 4 or Table 5. The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. This embodiment is illustrated in FIG. 3C with the amino acid modifications set forth in Table 4.

In one embodiment, the bispecific antibodies according to the invention are bivalent bispecific antibodies comprising one Fab fragment of an anti-CD3 antibody, one Fab fragment of an anti-BCMA antibody and one Fc part according to the format Fc-BCMA Fab-CD3 Fab. The anti-CD3 Fab fragment comprises (a) a light chain and heavy chain, wherein the light chain is a crossover light chain that comprises a variable domain VH and a constant domain CL, and wherein the heavy chain is a crossover heavy chain that comprises a variable domain VL and a constant domain CH1; and also (b) the amino acid modifications set forth in Table 4 or Table 5. This embodiment is illustrated in FIG. 3D with the amino acid modifications set forth in Table 4.

In one embodiment, the antibodies illustrated in FIG. 2 additionally comprise the modifications set forth in Table 2 or Table 3. For example, the antibodies illustrated in FIG. 2 may comprise the modifications set forth in Table 4 in combination with the modifications set forth in Table 2.

Alternatively, the antibodies illustrated in FIG. 2 may comprise the modifications set forth in Table 5 in combination with the modifications set forth in Table 3.

In one embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab.

The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. Each anti-BCMA Fab fragment comprises a light chain and heavy chain, wherein the heavy chain comprises a CH1 domain which comprises the amino acid modifications K147E and K213E (numbered according to EU numbering) and wherein the light chain comprises a corresponding CL domain which comprises the amino acid modifications E123R and Q124K (numbered according to Kabat) (i.e. the modifications set forth in Table 4). The Fc part comprises a first Fc chain and a second Fc chain, wherein the first Fc chain comprises a first constant domain CH2 and a first constant domain CH3, and the second Fc chain comprises a second constant domain CH2 and a second constant domain CH3. The first Fc chain is bound at the N-terminus of the Fc to the C-terminus of the first anti-BCMA Fab, and the second Fc chain is bound at the N-terminus of the Fc to the C-terminus of the anti-CD3 Fab. The first CH3 domain comprises the modifications T366S, L368A, and Y407V (“hole modifications”) and the second CH3 domain comprises the modification T366W (“knob modification”) (numbered according to EU numbering) (i.e. the modifications set forth in Table 2). Additionally, both Fc chains further comprise the modifications L234A, L235A and P329G, and optionally D356E and L358M (numbered according to EU numbering). Optionally, the first CH3 domain further comprises the amino acid modification S354C, and the second CH3 domain further comprises the amino acid modification Y349C (numbered according to EU numbering) such that a disulphide bridge between both CH3 domains is formed.

In another embodiment, the bispecific antibodies according to the invention are trivalent bispecific antibodies comprising one Fab fragment of an anti-CD3 antibody, two Fab fragments of an anti-BCMA antibody and one Fc part according to the format BCMA Fab-Fc-CD3 Fab-BCMA Fab.

The anti-CD3 Fab fragment comprises a light chain and heavy chain, wherein the light chain is a crossover light chain that comprises a variable domain VH and a constant domain CL, and wherein the heavy chain is a crossover heavy chain that comprises a variable domain VL and a constant domain CH1. Each anti-BCMA Fab fragment comprises a light chain and heavy chain, wherein the heavy chain comprises a CH1 domain which comprises the amino acid modifications A141W, L145E, K147T and Q175E (numbered according to EU numbering) and wherein the light chain comprises a corresponding CL domain which comprises the amino acid modifications F116A, Q124R, L135V and T178R (numbered according to Kabat numbering) (i.e. the modifications set forth in Table 5). The Fc part comprises a first Fc chain and a second Fc chain, wherein the first Fc chain comprises a first constant domain CH2 and a first constant domain CH3, and the second Fc chain comprises a second constant domain CH2 and a second constant domain CH3. The first CH3 domain comprises the modifications T350V, L351Y, F405A and Y407V and the second CH3 domain comprises the modifications T350V, T366L, K392L and T394W (numbered according to EU numbering) (i.e. the modifications set forth in Table 3). Additionally, both Fc chains further comprise the modifications L234A, L235A and P329G, and optionally D356E and L358M (numbered according to EU numbering).

In some embodiments, the anti-BCMA Fab fragment comprises a CDR1H, CDR2H, CDR3H, CDR1L, CDR2L and CDR3L region combination selected from the group of:
a) CDR1H region of SEQ ID NO:21, CDR2H region of SEQ ID NO:22, CDR3H region of SEQ ID NO:17, CDR1L region of SEQ ID NO:23, CDR2L region of SEQ ID NO:24, and a CDR3L region of SEQ ID NO:20,
b) CDR1H region of SEQ ID NO:21, CDR2H region of SEQ ID NO:22, CDR3H region of SEQ ID NO:17, CDR1L region of SEQ ID NO:25, CDR2L region of SEQ ID NO:26, and a CDR3L region of SEQ ID NO:20,
c) CDR1H region of SEQ ID NO:21, CDR2H region of SEQ ID NO:22, CDR3H region of SEQ ID NO:17, CDR1L region of SEQ ID NO:27, CDR2L region of SEQ ID NO:28, and a CDR3L region of SEQ ID NO:20,
d) CDR1H region of SEQ ID NO:29, CDR2H region of SEQ ID NO:30, CDR3H region of SEQ ID NO:17, CDR1L region of SEQ ID NO:31, CDR2L region of SEQ ID NO:32, and a CDR3L region of SEQ ID NO:33,
e) CDR1H region of SEQ ID NO:34, CDR2H region of SEQ ID NO:35, CDR3H region of SEQ ID NO:17, CDR1L region of SEQ ID NO:31, CDR2L region of SEQ ID NO:32, and a CDR3L region of SEQ ID NO:33,
f) CDR1H region of SEQ ID NO:36, CDR2H region of SEQ ID NO:37, CDR3H region of SEQ ID NO:17, CDR1L region of SEQ ID NO:31, CDR2L region of SEQ ID NO:32, and a CDR3L region of SEQ ID NO:33,
g) CDR1H region of SEQ ID NO:15, CDR2H region of SEQ ID NO:16, CDR3H region of SEQ ID NO:17, CDR1L region of SEQ ID NO:18, and CDR2L region of SEQ ID NO:19, and a CDR3L region of SEQ ID NO:20, and
   the anti-CD3 Fab fragment comprises a CDR1H region of SEQ ID NO:1, a CDR2H region of SEQ ID NO:2, a CDR3H region of SEQ ID NO:3, a CDR1L region of SEQ ID NO:4, a CDR2L region of SEQ ID NO:5 and a CDR3L region of SEQ ID NO:6.

In some embodiments, the anti-BCMA Fab fragment comprises a VH and a VL selected from the group consisting of:
a) a VH region of SEQ ID NO:10 and a VL region of SEQ ID NO:12,
b) a VH region of SEQ ID NO:10 and a VL region of SEQ ID NO:13,
c) a VH region of SEQ ID NO:10 and a VL region of SEQ ID NO:14,
d) a VH region of SEQ ID NO:38 and a VL region of SEQ ID NO:12,
e) a VH region of SEQ ID NO:39 and a VL region of SEQ ID NO:12,
f) a VH region of SEQ ID NO:40 and a VL region of SEQ ID NO:12, or
g) a VH region of SEQ ID NO:9 and a VL region of SEQ ID NO:11; and
   the anti-CD3 Fab fragment comprises a VH region of SEQ ID NO:7 and a VL region of SEQ ID NO:8.

In further embodiments, the multispecific (e.g. bispecific antibody) according to the invention comprises the following SEQ ID NOs (as mentioned in Tables 6A, 7B and 7C below):
83A10-TCBcv: 45, 46, 47 (×2), 48 (FIG. 2A)
21-TCBcv: 49, 50, 51 (×2), 48 (FIG. 2A)
22-TCBcv: 52, 53, 54 (×2), 48 (FIG. 2A)
42-TCBcv: 55, 56, 57 (×2), 48 (FIG. 2A)
Mab101: 58, 59, 60 (×2), 48 (FIG. 2A but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated)
Mab102: 61, 62, 63 (×2), 48 (FIG. 2A but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated)
Mab103: 64, 65, 66 (×2), 48 (FIG. 2A but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated).

The term “83A10-TCBcv” as used herein refers to a bispecific antibody specifically binding to BCMA and CD3 as specified by its heavy and light chain combination of SEQ ID NO:45, SEQ ID NO:46, SEQ ID NO:47 (2×), and SEQ ID NO:48, and as shown in FIG. 2A and described in EP14179705.

The terms “21-TCBcv, 22-TCBcv, 42-TCBcv” as used herein refer to the respective bispecific antibodies of Mab21, as specified by its heavy and light chain combination of SEQ ID NO:48, SEQ ID NO:49, SEQ ID NO:50, and SEQ ID NO:51 (2×), Mab22 as specified by its heavy and light chain combinations of SEQ ID NO:48, SEQ ID NO:52, SEQ ID NO:53, and SEQ ID NO:54 (2×), and Mab42 as specified by its heavy and light chain combination of SEQ ID NO:48, SEQ ID NO:55, SEQ ID NO:56, and SEQ ID NO:57-(2×), and as shown in FIG. 2A and described in WO 2017/021450.

The term “Mab101” as used herein refers to a bispecific antibody specifically binding to BCMA and CD3 as specified by its heavy and light chain combination of SEQ ID NO:48, SEQ ID NO:58, SEQ ID NO:60 (2×), and SEQ ID NO:59, and as shown in FIG. 2A (but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated). The term “Mab102” as used herein refers to a bispecific antibody specifically binding to BCMA and CD3 as specified by its heavy and light chain combination of SEQ ID NO:48, SEQ ID NO:61, SEQ ID NO:63 (2×), and SEQ ID NO:62, and as shown in FIG. 2A (but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated). The term “Mab103” as used herein refers to a bispecific antibody specifically binding to BCMA and CD3 as specified by its heavy and light chain combination of SEQ ID NO:48, SEQ ID NO:64, SEQ ID NO:66 (2×), and SEQ ID NO:65, and as shown in FIG. 2A (but with alternative amino acid substitutions in CL-CH1 to reduce light chain mispairing/side products: A141W, L145E, K147T, Q175E (“WETE”) and F, 116A, Q124R, L135V, T178R (“ARVR”) rather than the “RK/EE” substitutions illustrated).

In preferred embodiments, the bispecific antibody according to the invention is 42-TCBcv. The term “CC-93269” as used herein refers to the bispecific antibody 42-TCBcv.

Antibodies with Improved Stability

Provided herein are multispecific (e.g. bispecific) antibodies against BCMA and a T-cell antigen (e.g. CD3) having one or more amino acid modification(s) which provide improved stability (e.g. improved physiochemical properties) compared to antibodies without these modification(s). Also provided are nucleic acid molecules, vectors, host cells and pharmaceutical compositions comprising the same, methods of preparing the same and uses of the same including methods of treatment.

In one aspect of the invention, there is provided a multispecific antibody that binds to BCMA and a T-cell antigen, wherein the multispecific antibody comprises (i) an anti-BCMA antibody or antigen binding fragment thereof; (ii) an anti-T cell antigen antibody or antigen binding fragment thereof; and (iii) an Fc, wherein the anti-BCMA antibody or antigen binding fragment thereof comprises:
a) a VH domain comprising a CDR1H region of SEQ ID NO:21, CDR2H region of SEQ ID NO:22, CDR3H region of SEQ ID NO:17 and a VL domain comprising a CDR1L region of SEQ ID NO:27, CDR2L region of SEQ ID NO:28, and CDR3L region of SEQ ID NO:20;
b) a VH domain comprising a CDR1H region of SEQ ID NO:21, CDR2H region of SEQ ID NO:22, CDR3H region of SEQ ID NO:17 and a VL domain comprising a CDR1L region of SEQ ID NO:25, CDR2L region of SEQ ID NO:26, and CDR3L region of SEQ ID NO:20; or
c) a VH domain comprising a CDR1H region of SEQ ID NO:15, CDR2H region of SEQ ID NO:16, CDR3H region of SEQ ID NO:17 and a VL domain comprising a CDR1L region of SEQ ID NO:18, CDR2L region of SEQ ID NO:19, and CDR3L region of SEQ ID NO:20,
   wherein the multispecific antibody comprises a CH1 domain and a CL domain, wherein the CH1 domain comprises amino acid modifications at positions A141, L145, K147 and Q175 (numbered according to EU numbering) and the CL domain comprises amino acid modifications at positions F, 116, Q124, L135 and T178 (numbered according to Kabat),
   and wherein the Fc comprises a first Fc chain comprising first constant domains CH2 and CH3, and a second Fc chain comprising second constant domains CH2 and CH3, wherein the first CH3 domain comprises amino acid modifications at positions T350, L351, F405 and Y407 (numbered according to EU numbering) and the second CH3 domain comprises amino acid modifications at positions T350, T366, K392 and T394 (numbered according to EU numbering), optionally wherein the T cell antigen is CD3.

In some embodiments, the CH1 domain comprises two or more (e.g. all) of the modifications A141W, L145E, K147T and Q175E, or conservative substitutions thereof (numbered according to EU numbering), and wherein the CL domain comprises two or more (e.g. all) of the modifications F116A, Q124R, L135V and T178R, or conservative substitutions thereof (numbered according to Kabat).

The CH1 domain and the CL domain containing the amino acid modifications may be from the anti-BCMA antibody or antigen binding fragment thereof or the anti-T cell antigen antibody or antigen binding fragment thereof. In preferred embodiments, the anti-BCMA antibody or antigen binding fragment thereof comprises the CH1 domain and the CL domain comprising the amino acid modifications.

In some embodiments, the first CH3 domain comprises one or more of the modifications T350V, L351Y, F405A and Y407V, or conservative substitutions thereof (numbered according to EU numbering); and the second CH3 domain comprises one or more of the modifications T350V, T366L, K392L and T394W, or conservative substitutions thereof (numbered according to EU numbering). In preferred embodiments, the first CH3 domain comprises the modifications T350V, L351Y, F405A and Y407V, or conservative substitutions thereof (numbered according to EU numbering); and the second CH3 domain comprises the modifications T350V, T366L, K392L and T394W, or conservative substitutions thereof (numbered according to EU numbering).

In some embodiments, the multispecific (e.g. bispecific) antibodies of the invention comprise an anti-CD3 antibody, or antigen binding fragment thereof, wherein the VH domain of the anti-CD3 antibody comprises the CDRs of SEQ ID NO: 1, 2 and 3 as respectively CDRH1, CDRH2 and CDRH3 and the VL domain of the anti-CD3 antibody comprises the CDRs of SEQ ID NO: 4, 5 and 6 as respectively light chain CDRL1, CDRL2 and CDRL3.

In preferred embodiments, the multispecific (e.g. bispecific) antibodies of the invention comprise an anti-CD3 antibody or antigen binding fragment thereof comprising a VH of SEQ ID NO: 7 and a VL of SEQ ID NO: 8.

In some embodiments, the multispecific (e.g. bispecific) antibodies of the invention comprise an IgG1 Fc, and optionally wherein the Fc comprises:
a) the modifications L234A, L235A and P329G (numbered according to EU numbering); and/or
b) the modifications D356E, and L358M (numbered according to EU numbering).

In an embodiment, the multispecific antibody of the invention is a bispecific trivalent antibody comprising two Fab fragments of an anti-BCMA antibody and one Fab fragment of an anti-CD3 antibody, optionally wherein the antibody is in the format BCMA Fab-Fc-CD3 Fab-BCMA Fab.

In an aspect of the invention, there is provided a multispecific antibody that binds to BCMA and CD3, wherein the multispecific antibody is in the format of a trivalent bispecific antibody, wherein the multispecific antibody comprises one Fab fragment of an anti-CD3 antibody, two Fab fragments of an anti-BCMA antibody and one Fc, according to the format BCMA Fab-Fc-CD3 Fab-BCMA Fab, and wherein:
a) the anti-CD3 Fab fragment comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1,
b) each anti-BCMA Fab fragment comprises a light chain and a heavy chain, wherein the light chain comprises a variable domain VL and a constant domain CL, and the heavy chain comprises a variable domain VH and a constant domain CH1, and wherein:
   (i) the variable domain VH comprises heavy chain CDRs 1-3 of SEQ ID NOS: 21, 22 and 17, respectively and the variable domain VL comprises light chain CDRs 1-3 of SEQ ID NOS: 27, 28 and 20, respectively;
   (ii) the variable domain VH comprises heavy chain CDRs 1-3 of SEQ ID NOS: 21, 22, and 17, respectively, and the variable domain VL comprises light chain CDRs 1-3 of SEQ ID NOS: 25, 26 and 20, respectively; or
   (iii) the variable domain VH comprises heavy chain CDRs 1-3 of SEQ ID NOS: 15, 16, and 17, respectively, and the variable domain VL comprises light chain CDRs 1-3 of SEQ ID NOS: 18, 19 and 20, respectively;
c) the CH1 domain of each anti-BCMA Fab fragment comprises the modifications A141W, L145E, K147T and Q175E or conservative substitutions thereof (numbered according to EU numbering) and the corresponding CL domain of each anti-BCMA Fab fragment comprises the modifications F116A, Q124R, L135V, T178R or conservative substitutions thereof (numbered according to Kabat numbering),
d) the Fc comprises a first Fc chain and a second Fc chain, the first Fc chain comprising first constant domains CH2 and CH3, and the second Fc chain comprising second constant domains CH2 and CH3, wherein the first Fc chain is bound at the N-terminus of the Fc to the C-terminus of one anti-BCMA Fab fragment, and the second Fc chain is bound at the N-terminus of the Fc to the C-terminus of the anti-CD3 Fab fragment, and wherein:
   (i) the first CH3 domain comprises the modifications T350V, L351Y, F405A and Y407V and the second CH3 domain comprises the modifications T350V, T366L, K392L and T394W (numbered according to EU numbering), and
   (ii) both Fc chains comprise the modifications L234A, L235A and P329G, and optionally the modifications D356E and L358M (numbered according to EU numbering).

In a further aspect of the invention, there is provided a trivalent bispecific antibody that binds to BCMA and to CD3, wherein the trivalent bispecific antibody comprises the following SEQ ID NOs:
i. Mab101: 58, 59, 48, and 2×60.
ii. Mab102: 61, 62, 48, and 2×63.
iii. Mab103: 64, 65, 48, and 2×66.

Each molecule Mab101, Mab102 and Mab103 is in a 2+1 bispecific format as shown in FIG. 2A but with alternative amino acid substitutions in CL-CH1 to reduce light chain mispairing/side products: A141W, L145E, K147T, Q175E (“WETE”) and F116A, Q124R, L135V, T178R (“ARVR”) rather than the “RK/EE” substitutions illustrated.

In a further aspect, there is provided a pharmaceutical composition comprising the multispecific (e.g. bispecific) antibody of the invention and a pharmaceutically acceptable excipient. In a related aspect, there is provided a pharmaceutical composition comprising the trivalent bispecific antibody of the invention and a pharmaceutically acceptable excipient.

In a further aspect, the multispecific (e.g. bispecific) antibody of the invention, the trivalent bispecific antibody of the invention or the pharmaceutical composition of the invention is for use as a medicament.

In a related aspect, there is provided a method of treating a subject, the method comprising administering to a subject (e.g. a human) in need of such treatment the multispecific (e.g. bispecific) antibody of the invention, the trivalent bispecific antibody of the invention or the pharmaceutical composition of the invention.

In preferred embodiments, the multispecific (e.g. bispecific) antibody of the invention, the trivalent bispecific antibody of the invention or the pharmaceutical composition of the invention is for use as a medicament for the treatment of a plasma cell disorder. In some embodiments, the plasma cell disorder is a cancer. In preferred embodiments, the cancer is multiple myeloma or plasma cell leukemia.

Pharmaceutical Compositions

The multispecific (e.g. bispecific) antibodies of the invention can be administered to the subject as a pharmaceutical composition. Accordingly, the present invention also provides a pharmaceutical composition comprising the multispecific (e.g. bispecific) antibodies of the invention and a pharmaceutically acceptable excipient.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

The pharmaceutical compositions disclosed herein are for use in, but not limited to, diagnosing, detecting, or monitoring a disorder, in preventing, treating, managing, or ameliorating a disorder or one or more symptoms thereof, and/or in research. The pharmaceutical compositions disclosed herein may be suitable for veterinary uses or pharmaceutical uses in humans.

Examples of suitable excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable excipients include: (1) Dulbecco's phosphate buffered saline, pH.about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

A person skilled in the art would understand that the appropriate choice of excipient or excipients for use with multispecific (e.g. bispecific) antibodies of the invention would depend on the desired properties of the pharmaceutical composition.

The pharmaceutical compositions or the multispecific (e.g. bispecific) antibodies of the invention can be administered to a subject by any appropriate systemic or local route of administration. For example, administration may be oral, buccal, sublingual, ophthalmic, intranasal, intratracheal, pulmonary, topical, transdermal, urogenital, rectal, subcutaneous, intravenous, intra-arterial, intraperitoneal, intramuscular, intracranial, intrathecal, epidural, intraventricular or intratumoral. In some embodiments, the pharmaceutical compositions or the multispecific (e.g. bispecific) antibody is administered intravenously or subcutaneously. In preferred embodiments, the pharmaceutical compositions or the multispecific (e.g. bispecific) antibody is administered intravenously.

Pharmaceutical compositions of the invention can be formulated for administration by any appropriate means, for example by epidermal or transdermal patches, ointments, lotions, creams, or gels; by nebulizers, vaporisers, or inhalers; by injection or infusion; or in the form of capsules, tablets, liquid solutions or suspensions in water or non-aqueous media, drops, suppositories, enemas, sprays, or powders. The most suitable route for administration in any given case will depend on the physical and mental condition of the patient, the nature and severity of the disease, and the desired properties of the formulation.

Monotherapies and Combination Therapies

In some embodiments, the treatment comprises the administration of the multispecific (e.g. bispecific) antibody of the invention to the subject as a monotherapy.

In some embodiments, the treatment comprises the administration of the multispecific (e.g. bispecific) antibody of the invention to the subject as a combination therapy, wherein the combination therapy comprises the administration of the multispecific (e.g. bispecific) antibody of the invention and one or more additional therapeutic agents. The term “combination therapy” is meant to encompass administration of the selected therapeutic agents to a single patient, and is intended to include treatments in which the agents are administered by the same or different route of administration or at the same or different time.

In some embodiments, the one or more additional therapeutic agents are selected from the group consisting of an antifolate (e.g. methotrexate), an inhibitor of purine synthesis (e.g. azathioprine, mycophenolate and/or mycophenolate mofetil), a C5a inhibitor (e.g. avacopan), an anti-CD19 antibody, an anti-CD20 antibody (e.g. rituximab), a steroid, a Bruton's tyrosine kinase (BTK) inhibitor and/or a BAFF/APRIL antagonist (e.g. an anti-BAFF antibody).

The present inventors have identified that there is minimal need for steroids in remission induction and/or maintenance of remission. Thus, in some embodiments, the one or more additional therapeutic agents is not a steroid e.g. a glucocorticoid.

In some embodiments, the one or more additional therapeutic agents are selected from the group consisting of thalidomide and an immunotherapeutic derivative thereof, an anti-CD38 antibody, an anti-PD-1 antibody, an anti-PD-L1 antibody, a gamma secretase inhibitor (GSI), an anti-BCMA antibody drug conjugate and anti-BCMA CAR T-cell therapy.

The term “anti-CD38 antibody” as used herein relates to an antibody specifically binding to human CD38. In an embodiment of the invention the anti-CD38 antibody is daratumumab (US20150246123). In an embodiment of the invention the anti-CD38 antibody is isatuximab (SAR650984, U.S. Pat. No. 8,877,899). In an embodiment of the invention the anti-CD38 antibody is MOR202 (WO 2012041800). In an embodiment of the invention the anti-CD38 antibody is Ab79 (U.S. Pat. No. 8,362,211). In an embodiment of the invention the anti-CD38 antibody is Ab19 (U.S. Pat. No. 8,362,211). The dosage of such anti-CD38 antibody is performed according to the state of the art and described in the respective prescribing informations. E.g. Daratumumab dosage is usually 16 mg/kg (www.ema.europa.eu).

The term “thalidomide compound” or “thalidomide and an immunotherapeutic derivative” as used herein relates to 2-(2,6-dioxopiperidin-3-yl)-2,3-dihydro-1H-isoindole-1,3-dione and immunotherapeutic derivatives thereof. In an embodiment of the invention the thalidomide compound is selected from the group consisting of, but not limited to, thalidomide (CAS Registry Number 50-35-1), lenalidomide (CAS Registry Number 191732-72-6), pomalidomide (CAS Registry Number 19171-19-8), CC122 (CAS Registry Number 1398053-45-6) and CC-220 (CAS Registry Number 1323403-33-3) and the respective salts (preferably HCl salts 1:1). The chemical formula of CC-122 is 2,6-piperidinedione, 3-(5-amino-2-methyl-4-oxo-3(4H-quinazolinyl), hydrochloride (1:1) and of CC-220 it is 2,6-piperidinedione, 3-[1,3-dihydro-4-[[4-(4-morpholinylmethyl)phenyl]methoxy]-1-oxo-2H-isoindol-2-yl]-, (3S)-, hydrochloride (1:1). Methods of preparing CC-220 are described, e.g., in US 20110196150, the entirety of which is incorporated herein by reference.

The dosage of thalidomide compounds is performed according to the state of the art and described in the respective prescribing informations. E.g. Revlimid® (lenalidomide) dosage is usually 25 mg once daily orally on days 1-21 of repeated 28-day cycles (www.revlimid.com) and POMALYST® (pomalidomide) dosage for the treatment of Multiple Myeloma is usually 4 mg per day taken orally on days 1-21 of repeated 28-day cycles (www.celgene.com). In one embodiment, 3-(5-amino-2-methyl-4-oxo-4H-quinazolin-3-yl)-piperidine-2,6-dione is administered in an amount of about 5 to about 50 mg per day.

In one embodiment, CC-122 and CC-220 are administered in an amount of about 5 to about 25 mg per day. In another embodiment, CC-122 and CC-220 are administered in an amount of about 5, 10, 15, 25, 30 or 50 mg per day. In another embodiment, 10 or 25 mg of CC-122 and CC-220 are administered per day. In one embodiment, CC-122 and CC-220 are administered twice per day.

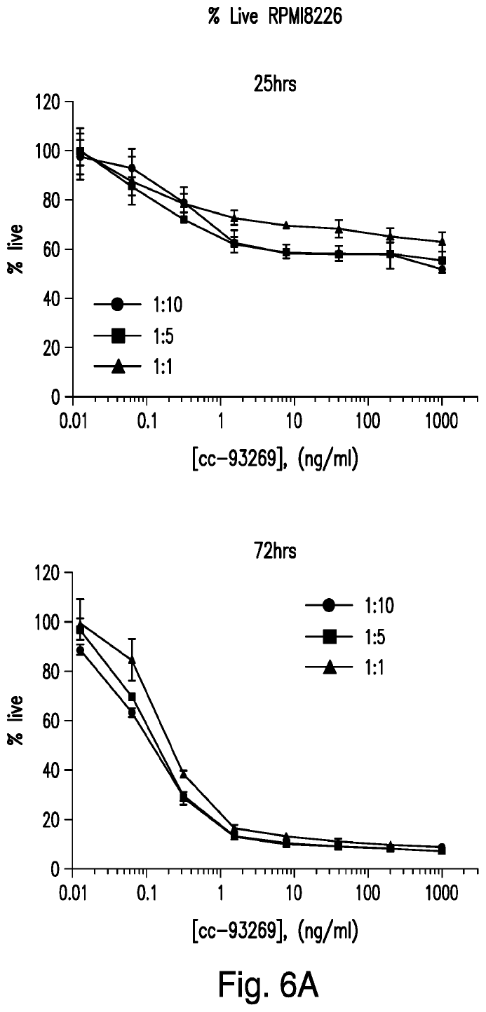

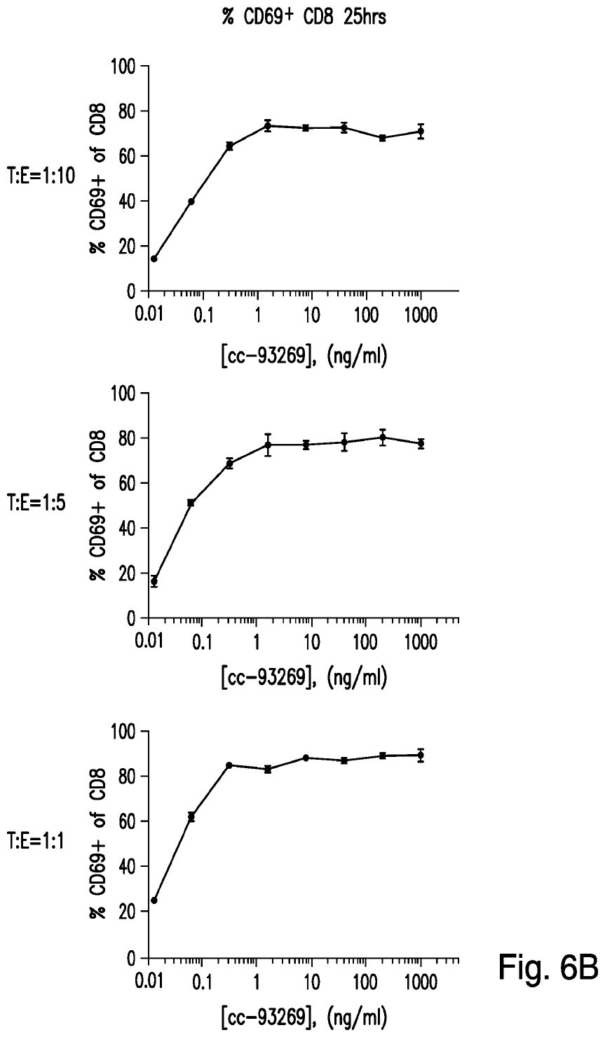

The term “anti-PD-1 antibody” as used herein relates to an antibody specifically binding to human PD-1. Such antibodies are e.g. described in WO2015026634 (MK-3475, pembrolizumab), U.S. Pat. Nos. 7,521,051, 8,008,449, and 8,354,509. Pembrolizumab (Keytruda®, MK-3475) is also described in WO 2009/114335, Poole, R. M. Drugs (2014) 74: 1973; Seiwert, T., et al., J. Clin. Oncol. 32, 5s (suppl; abstr 6011). In an embodiment of the invention the PD-1 antibody is MK-3475 (WHO Drug Information, Vol. 27, No. 2, pages 161-162 (2013)) and which comprises the heavy and light chain amino acid sequences shown in FIG. 6 of WO 2015026634 The amino acid sequence of pembrolizumab is described in WO2008156712 (light chain CDRs SEQ ID NOS:15, 16 and 17 and heavy chain CDRs SEQ ID NOS: 18, 19 and 20). In an embodiment of the invention the PD-1 antibody is nivolumab (BMS-936558, MDX 1106; WHO Drug Information, Vol. 27, No. 1, pages 68-69 (2013), WO2006/121168 amino acid sequences shown in WO 2015026634). In an embodiment of the invention the PD-1 antibody is; pidilizumab (CT-011, also known as hBAT or hBAT-1; amino acid sequence see WO2003/099196; WO 2009/101611, Fried I. et al.; Neuro Oncol (2014) 16 (suppl 5): v111-v112). In an embodiment of the invention the PD-1 antibody is MEDI-0680 (AMP-514, WO2010/027423, WO2010/027827, WO2010/027828, Hamid O. et al.; J Clin Oncol 33, 2015 (suppl; abstr TPS3087). In an embodiment of the invention the PD-1 antibody is PDR001 (Naing A. et al.; J Clin Oncol 34, 2016 (suppl; abstr 3060). In an embodiment of the invention the PD-1 antibody is REGN2810 (Papadopoulos K P et al.; J Clin Oncol 34, 2016 (suppl; abstr 3024). In an embodiment of the invention the PD-1 antibody is lambrolizumab (WO2008/156712). In an embodiment of the invention the PD-1 antibody is h409A1 1, h409A16 or h409A17, which are described in WO2008/156712. The dosage of such anti-PD-1 antibody is performed according to the state of the art and described in the respective prescribing informations. E.g. Keytruda® is administered usually in a concentration of 2 mg/kg body weight every three weeks (http://ec.europa.eu/health/documents).

The term “anti-PD-L1 antibody” as used herein relates to an antibody specifically binding to human PD-L1. Such antibodies are e.g. described in WO2015026634, WO2013/019906, WO2010/077634 and U.S. Pat. No. 8,383,796. In an embodiment of the invention the PD-L1 antibody is MPDL3280A (atezolizumab, YW243.55.S70, WO2010/077634, McDermott D F. Et al., JCO Mar. 10, 2016 vol. 34 no. 8 833-842). In an embodiment of the invention the PD-L1 antibody is MDX-1105 (BMS-936559, WO2007/005874, Patrick A. Ott P A et al., DOI: 10.1158/1078-0432, Clinical Cancer Research-13-0143). In an embodiment of the invention the PD-L1 antibody is MEDI4736 (durvalumab, WO 2016/040238 Gilbert J. et al., Journal for ImmunoTherapy of Cancer 20153(Suppl 2):P152). In an embodiment of the invention the PD-L1 antibody is MSB001071 8C (avelumab, Disis M L. et al., Journal of Clinical Oncology, Vol 33, No 15_suppl (May 20 Supplement), 2015: 5509). In an embodiment of the invention the PD-L1 antibody is the anti-PD-L1 antibody comprising a VH sequence of SEQ ID NO: 16 and a VL sequence of SEQ ID NO: 17 as described in WO2016007235. The dosage of such anti-PD-L1 antibody is performed according to the state of the art and described in the respective prescribing informations. E.g. atezolizumab is administered usually in a concentration of 1200 mg as an intravenous infusion over 60 minutes every 3 weeks (www.accessdata.fda.gov).

The term “gamma secretase” as used herein refers to any protein or protein complex that exhibits gamma secretase activities including binding to a substrate having a gamma secretase cleavage sequence, and catalyzing the cleavage of the gamma secretase cleavage sequence, at a gamma secretase cleavage site, to produce substrate cleavage products. In one embodiment, gamma secretase is a protein complex comprising one or more of the following subunits: presenilin, nicastrin, gamma-secretase subunit APH-1, and gamma-secretase subunit PEN-2.

The term “gamma secretase inhibitor” or “GSI” as used herein refers to any molecule capable of inhibiting or reducing expression and/or function of gamma secretase. In certain embodiment, the GSI reduces expression and/or function of a subunit of gamma secretase (e.g., presenilin, nicastrin, APH-1, or PEN-2). Any form of a “gamma secretase inhibitor” such as a salt, a co-crystal, a crystalline form, a pro-drug, etc., is included within this term. In some embodiments, the GSI is selected from an antibody or antigen-binding fragment, a small molecule, a protein or peptide and a nucleic acid.

Adverse Events

In some embodiments, the patient develops, or is at risk of developing, an adverse event associated with the administration of the multispecific (e.g. bispecific) antibody. The adverse event may be cytokine-driven toxicities (e.g. cytokine release syndrome (CRS)), infusion-related reactions (IRRs), infection, macrophage activation syndrome (MAS), neurologic toxicities, severe tumor lysis syndrome (TLS), neutropenia, thrombocytopenia, elevated liver enzymes, and/or central nervous system (CNS) toxicities. In particular embodiments, the adverse event is CRS.

In the event that the patient develops, or is at risk of developing, an adverse event associated with the administration of the multispecific (e.g. bispecific) antibody, the treatment may further comprise the administration of an agent capable of treating, preventing, delaying, reducing or attenuating the development or risk of development of the adverse event. The agent may be administered to the patient prior to the initiation of the treatment with the multispecific (e.g. bispecific) antibody (e.g. as a prophylaxis in order to prevent or reduce the risk of an adverse event developing) or during treatment with the multispecific (e.g. bispecific) antibody (e.g. in response to the development of an adverse event).

In some embodiments, the agent comprises a steroid, such as a corticosteroid. As used herein, “corticosteroid” means any naturally occurring or synthetic steroid hormone that can be derived from cholesterol and is characterized by a hydrogenated cyclopentanoperhydrophenanthrene ring system. Naturally occurring corticosteroids are generally produced by the adrenal cortex. Synthetic corticosteroids may be halogenated. Functional groups required for activity include a double bond at A4, a C3 ketone, and a C20 ketone. Corticosteroids may have glucocorticoid and/or mineralocorticoid activity. Examples of exemplary corticosteroids include prednisolone, methylprednisolone, prednisone, triamcinolone, betamethasone, budesonide, and dexamethasone.

In some embodiments, the agent comprises an antagonist of a cytokine receptor or cytokine selected from among GM-CSF, IL-10, IL-10R, IL-6, IL-6 receptor (IL-6R), IFNγ, IFNGR, IL-2, IL-2R/CD25, MCP-1, CCR2, CCR4, MIPIβ, CCR5, TNFalpha, TNFR1, IL-1, and IL-1Ralpha/IL-1beta, wherein the antagonist is selected from an antibody or antigen-binding fragment, a small molecule, a protein or peptide and a nucleic acid. The antagonist may be an anti-IL-6 antibody and/or an anti-IL6R antibody. For example, the antagonist may be selected from tocilizumab, siltuximab, clazakizumab, sarilumab, olokizumab, elsilimomab, ALD518/BMS-945429, sirukumab (CNTO 136), CPSI-2634, ARGX-109, lenzilumab, FE301 and FM101. In some embodiments, the antagonist is tocilizumab and/or siltuximab.

In some embodiments, the agent comprises a molecule that decreases the regulatory T cell (Treg) population. Agents that decrease the number of (e.g., deplete) Treg cells are known in the art and include, e.g., CD25 depletion, cyclophosphamide administration, anti-CTLA4 antibody and modulating Glucocorticoid-induced TNLR family related gene (GITR) function. GITR is a member of the TNLR superfamily that is upregulated on activated T cells, which enhances the immune system. In some embodiments, the treatment comprises the administration of cyclophosphamide.

As noted above, the present inventors have observed no significant or minimal cytokine release following treatment with the bispecific antibodies of the invention. Accordingly, in some embodiments in which the adverse event is a cytokine-driven toxicity (e.g. CRS), the treatment does not further comprise the administration of an agent capable of treating, preventing, delaying, reducing or attenuating the development or risk of development of the adverse event, such as an antagonist of a cytokine receptor or cytokine.

Disclaimer

The above embodiments are to be understood as illustrative examples. Further embodiments are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

In the context of the present invention other examples and variations of the antibodies and methods described herein will be apparent to a person of skill in the art. Other examples and variations are within the scope of the invention, as set out in the appended claims.

All documents cited herein are each entirely incorporated by reference herein, including all data, tables, figures, and text presented in the cited documents.

TABLE 6A
Antibody sequences

| SEQ ID NO: | Name(s) | Amino acid sequences |
|---|---|---|
| 1 | CD3 CDR1H | TYAMN |
| 2 | CD3 CDR2H | RIRSKYNNYATYYADSVKG |
| 3 | CD3 CDR3H | HGNFGNSYVSWFAY |
| 4 | CD3 CDR1L | GSSTGAVTTSNYAN |
| 5 | CD3 CDR2L | GTNKRAP |
| 6 | CD3 CDR3L | ALWYSNLWV |
| 7 | CD3 VH | EVQLLESGGGLVQPGGSLRLSCAASGFTFSTYAMNW |
| | | VRQAPGKGLEWVSRIRSKYNNYATYYADSVKGRFTI |
| | | SRDDSKNTLYLQMNSLRAEDTAVYYCVRHGNFGNS |
| | | YVSWFAYWGQGTLVTVSS |
| 8 | CD3 VL | QAVVTQEPSLTVSPGGTVTLTCGSSTGAVTTSNYAN |
| | | WVQEKPGQAFRGLIGGTNKRAPGTPARFSGSLLGGK |
| | | AALTLSGAQPEDEAEYYCALWYSNLWVFGGGTKLT |
| | | VL |
| 9 | 83A10 VH | EVQLLESGGGLVQPGGSLRLSCAASGFTFSSYAMSW |
| | | VRQAPGKGLEWVSAISGSGGSTYYADSVKGRFTISRD |
| | | NSKNTLYLQMNSLRAEDTAVYYCAKVLGWFDYWG |
| | | QGTLVTVSS |
| 10 | Mab21 VH | EVQLLESGGGLVQPGGSLRLSCAASGFTFSDNAMGW |
| | Mab22 VH | VRQAPGKGLEWVSAISGPGSSTYYADSVKGRFTISRD |
| | Mab42 VH | NSKNTLYLQMNSLRAEDTAVYYCAKVLGWFDYWG |
| | | QGTLVTVSS |
| 11 | 83A10 VL | EIVLTQSPGTLSLSPGERATLSCRASQSVSSSYLAWYQ |
| | | QKPGQAPRLLIYGASSRATGIPDRFSGSGSGTDFTLTIS |
| | | RLEPEDFAVYYCQQYGYPPDFTFGQGTKVEIK |
| 12 | Mab21 VL | EIVLTQSPGTLSLSPGERATLSCRASQSVSEYYLAWY |
| | Mab27 VL | QQKPGQAPRLLIEHASTRATGIPDRFSGSGSGTDFTLT |
| | Mab33 VL | ISRLEPEDFAVYYCQQYGYPPDFTFGQGTKVEIK |
| | Mab39 VL | |
| 13 | Mab22 VL | EIVLTQSPGTLSLSPGERATLSCRASQSVSSYYLAWY |
| | | QQKPGQAPRLLISGAGSRATGIPDRFSGSGSGTDFTLT |
| | | ISRLEPEDFAVYYCQQYGYPPDFTFGQGTKVEIK |
| 14 | Mab42 VL | EIVLTQSPGTLSLSPGERATLSCRASQSVSDEYLSWYQ |
| | | QKPGQAPRLLIHSASTRATGIPDRFSGSGSGTDFTLAIS |
| | | RLEPEDFAVYYCQQYGYPPDFTFGQGTKVEIK |
| 15 | 83A10 CDR1H | SYAMS |
| 16 | 83A10 CDR2H | AISGSGGSTYYADSVKG |
| 17 | 83A10 CDR3H | VLGWFDY |
| | Mab21 CDR3H | |
| | Mab22 CDR3H | |
| | Mab42 CDR3H | |
| | Mab27 CDR3H | |
| | Mab33 CDR3H | |
| | Mab39 CDR3H | |
| 18 | 83A10 CDR1L | RASQSVSSSYLA |
| 19 | 83A10 CDR2L | GASSRAT |
| 20 | 83A10 CDR3L | QQYGYPPDFT |
| | Mab21 CDR3L | |
| | Mab22 CDR3L | |
| | Mab42 CDR3L | |
| 21 | Mab21 CDR1H | DNAMG |
| | Mab22 CDR1H | |
| | Mab42 CDR1H | |
| 22 | Mab21 CDR2H | AISGPGSSTYYADSVKG |
| | Mab22 CDR2H | |
| | Mab42 CDR2H | |
| 23 | Mab21 CDR1L | RASQSVSEYYLAW |
| 24 | Mab21 CDR2L | EHASTRAT |
| 25 | Mab22 CDR1L | RASQSVSSYYLA |
| 26 | Mab22 CDR2L | GAGSRAT |
| 27 | Mab42 CDR1L | RASQSVSDEYLS |
| 28 | Mab42 CDR2L | SASTRAT |
| 29 | Mab27 CDR1H | SAPMG |
| 30 | Mab27 CDR2H | AISYIGHTYYADSVKG |
| 31 | Mab27 CDR1L | RASQSVSEYYLA |
| | Mab33 CDR1L | |
| | Mab39 CDR1L | |
| 32 | Mab27 CDR2L | HASTRAT |
| | Mab33 CDR2L | |
| | Mab39 CDR2L | |
| 33 | Mab27 CDR3L | QQYGYPPDFT |
| | Mab33 CDR3L | |
| | Mab39 CDR3L | |
| 34 | Mab33 CDR1H | TNAMG |
| 35 | Mab33 CDR2H | AINRFGGSTYYADSVKG |
| 36 | Mab39 CDR1H | QNAMG |
| 37 | Mab39 CDR2H | AISPTGFSTYYADSVKG |
| 38 | Mab27 VH | EVQLLESGGGLVQPGGSLRLSCAASGFTFSSAPMGW |
| | | VRQAPGKGLEWVSAISYIGHTYYADSVKGRFTISRDN |
| | | SKNTLYLQMNSLRAEDTAVYYCAKVLGWFDYWGQ |
| | | GTLVTVSS |
| 39 | Mab33 VH | EVQLLESGGGLVQPGGSLRLSCAASGFTFYTNAMGW |
| | | VRQAPGKGLEWVSAINRFGGSTYYADSVKGRFTISR |
| | | DNSKNTLYLQMNSLRAEDTAVYYCAKVLGWFDYW |
| | | GQGTLVTVSS |
| 40 | Mab39 VH | EVQLLESGGGLVQPGGSLRLSCAASGFTFTQNAMGW |
| | | VRQAPGKGLEWVSAISPTGFSTYYADSVKGRFTISRD |
| | | NSKNTLYLQMNSLRAEDTAVYYCAKVLGWFDYWG |
| | | QGTLVTVSS |
| 41 | 83A10 BCMA CH1 | ASTKGPSVFPLAPSSKSTSGGTAALGCLVEDYFPEPVT |
| | Mab21 BCMA CH1 | VSWNSGALTSGVHTFPAVLQSSGLYSLSSVVTVPSSS |
| | Mab22 BCMA CH1 | LGTQTYICNVNHKPSNTKVDEKVEPKSC |
| | Mab42 BCMA CH1 | |
| 42 | 83A10 BCMA CL | RTVAAPSVFIFPPSDRKLKSGTASVVCLLNNFYPREA |
| | Mab21 BCMA CL | KVQWKVDNALQSGNSQESVTEQDSKDSTYSLSSTLT |
| | Mab22 BCMA CL | LSKADYEKHKVYACEVTHQGLSSPVTKSFNRGEC |
| | Mab42 BCMA CL | |
| 43 | CD3 CH1 | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSC |
| 44 | CD3 CL | ASVAAPSVFIFPPSDEQLKSGTASVVCLLNNFYPREAK |
| | | VQWKVDNALQSGNSQESVTEQDSKDSTYSLSSTLTL |
| | | SKADYEKHKVYACEVTHQGLSSPVTKSFNRGEC |
| 45 | 83A10 knob HC | EVQLLESGGGLVQPGGSLRLSCAASGFTFSSYAMSW |
| | | VRQAPGKGLEWVSAISGSGGSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDGGGGGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYTLPPCRDE |
| | | LTKNQVSLWCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 46 | 83A10 hole HC | EVQLLESGGGLVQPGGSLRLSCAASGFTFSSYAMSW |
| | | VRQAPGKGLEWVSAISGSGGSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVCTLPPSRDELTKNQVSL |
| | | SCAVKGFYPSDIAVEWESNGQPENNYKTTPPVLDSD |
| | | GSFFLVSKLTVDKSRWQQGNVFSCSVMHEALHNHYT |
| | | QKSLSLSPGK |
| 47 | 83A10 LC | EIVLTQSPGTLSLSPGERATLSCRASQSVSSSYLAWYQ |
| | | QKPGQAPRLLIYGASSRATGIPDRFSGSGSGTDFTLTIS |
| | | RLEPEDFAVYYCQQYGYPPDFTFGQGTKVEIKRTVA |
| | | APSVFIFPPSDRKLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| 48 | CD3 LC | EVQLLESGGGLVQPGGSLRLSCAASGFTFSTYAMNW |
| | | VRQAPGKGLEWVSRIRSKYNNYATYYADSVKGRFTI |
| | | SRDDSKNTLYLQMNSLRAEDTAVYYCVRHGNFGNS |
| | | YVSWFAYWGQGTLVTVSSASVAAPSVFIFPPSDEQLK |
| | | SGTASVVCLLNNFYPREAKVQWKVDNALQSGNSQES |
| | | VTEQDSKDSTYSLSSTLTLSKADYEKHKVYACEVTH |
| | | QGLSSPVTKSFNRGEC |
| 49 | Mab21 knob HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYTLPPCRDE |
| | | LTKNQVSLWCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 50 | Mab21 hole HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVCTLPPSRDELTKNQVSL |
| | | SCAVKGFYPSDIAVEWESNGQPENNYKTTPPVLDSD |
| | | GSFFLVSKLTVDKSRWQQGNVFSCSVMHEALHNHYT |
| | | QKSLSLSPGK |
| 51 | Mab21 LC | EIVLTQSPGTLSLSPGERATLSCRASQSVSEYYLAWY |
| | | QQKPGQAPRLLIEHASTRATGIPDRFSGSGSGTDFTLT |
| | | ISRLEPEDFAVYYCQQYGYPPDFTFGQGTKVEIKRTV |
| | | AAPSVFIFPPSDRKLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 52 | Mab22 knob HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYTLPPCRDE |
| | | LTKNQVSLWCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 53 | Mab22 hole HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVCTLPPSRDELTKNQVSL |
| | | SCAVKGFYPSDIAVEWESNGQPENNYKTTPPVLDSD |
| | | GSFFLVSKLTVDKSRWQQGNVFSCSVMHEALHNHYT |
| | | QKSLSLSPGK |
| 54 | Mab22 LC | EIVLTQSPGTLSLSPGERATLSCRASQSVSSYYLAWY |
| | | QQKPGQAPRLLISGAGSRATGIPDRFSGSGSGTDFTLT |
| | | ISRLEPEDFAVYYCQQYGYPPDFTFGQGTKVEIKRTV |
| | | AAPSVFIFPPSDRKLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 55 | Mab42 knob HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYTLPPCRDE |
| | | LTKNQVSLWCLVKGFYPSDIAVEWESNGQPENNYKT |
| | | TPPVLDSDGSFFLYSKLTVDKSRWQQGNVFSCSVMH |
| | | EALHNHYTQKSLSLSPGK |
| 56 | Mab42 hole HC | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAALGCL |
| | | VEDYFPEPVTVSWNSGALTSGVHTFPAVLQSSGLYSL |
| | | SSVVTVPSSSLGTQTYICNVNHKPSNTKVDEKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVCTLPPSRDELTKNQVSL |
| | | SCAVKGFYPSDIAVEWESNGQPENNYKTTPPVLDSD |
| | | GSFFLVSKLTVDKSRWQQGNVFSCSVMHEALHNHYT |
| | | QKSLSLSPGK |
| 57 | Mab42 LC | EIVLTQSPGTLSLSPGERATLSCRASQSVSDEYLSWYQ |
| | | QKPGQAPRLLIHSASTRATGIPDRFSGSGSGTDFTLAIS |
| | | RLEPEDFAVYYCQQYGYPPDFTFGQGTKVEIKRTVA |
| | | APSVFIFPPSDRKLKSGTASVVCLLNNFYPREAKVQW |
| | | KVDNALQSGNSQESVTEQDSKDSTYSLSSTLTLSKAD |
| | | YEKHKVYACEVTHQGLSSPVTKSFNRGEC |
| 58 | Mab101 HC-1 | EVQLLESGGGLVQPGGSLRLSCAASGFTFSSYAMSW |
| | | VRQAPGKGLEWVSAISGSGGSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVYVYPPSREEMTKNQVS |
| | | LTCLVKGFYPSDIAVEWESNGQPENNYKTTPPVLDSD |
| | | GSFALVSKLTVDKSRWQQGNVFSCSVMHEALHNHY |
| | | TQKSLSLSPG |
| 59 | Mab101 HC-2 | EVQLLESGGGLVQPGGSLRLSCAASGFTFSSYAMSW |
| | | VRQAPGKGLEWVSAISGSGGSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYVLPPSREE |
| | | MTKNQVSLLCLVKGFYPSDIAVEWESNGQPENNYLT |
| | | WPPVLDSDGSFFLYSKLTVDKSRWQQGNVFSCSVMH |
| | | EALHNHYTQKSLSLSPG |
| 60 | Mab101 LC | EIVLTQSPGTLSLSPGERATLSCRASQSVSSSYLAWYQ |
| | | QKPGQAPRLLIYGASSRATGIPDRFSGSGSGTDFTLTIS |
| | | RLEPEDFAVYYCQQYGYPPDFTFGQGTKVEIKRTVA |
| | | APSVAIFPPSDERLKSGTASVVCVLNNFYPREAKVQW |
| | | KVDNALQSGNSQESVTEQDSKDSTYSLSSRLTLSKAD |
| | | YEKHKVYACEVTHQGLSSPVTKSFNRGEC |
| 61 | Mab102 HC-1 | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVYVYPPSREEMTKNQVS |
| | | LTCLVKGFYPSDIAVEWESNGQPENNYKTTPPVLDSD |
| | | GSFALVSKLTVDKSRWQQGNVFSCSVMHEALHNHY |
| | | TQKSLSLSPG |
| 62 | Mab102 HC-2 | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYVLPPSREE |
| | | MTKNQVSLLCLVKGFYPSDIAVEWESNGQPENNYLT |
| | | WPPVLDSDGSFFLYSKLTVDKSRWQQGNVFSCSVMH |
| | | EALHNHYTQKSLSLSPG |
| 63 | Mab102 LC | EIVLTQSPGTLSLSPGERATLSCRASQSVSSYYLAWY |
| | | QQKPGQAPRLLISGAGSRATGIPDRFSGSGSGTDFTLT |
| | | ISRLEPEDFAVYYCQQYGYPPDFTFGQGTKVEIKRTV |
| | | AAPSVAIFPPSDERLKSGTASVVCVLNNFYPREAKVQ |
| | | WKVDNALQSGNSQESVTEQDSKDSTYSLSSRLTLSK |
| | | ADYEKHKVYACEVTHQGLSSPVTKSFNRGEC |
| 64 | Mab103 HC-1 | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDKTHTCPPCPAPEAAGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LGAPIEKTISKAKGQPREPQVYVYPPSREEMTKNQVS |
| | | LTCLVKGFYPSDIAVEWESNGQPENNYKTTPPVLDSD |
| | | GSFALVSKLTVDKSRWQQGNVFSCSVMHEALHNHY |
| | | TQKSLSLSPG |
| 65 | Mab103 HC-2 | EVQLLESGGGLVQPGGSLRLSCAASGFTFSDNAMGW |
| | | VRQAPGKGLEWVSAISGPGSSTYYADSVKGRFTISRD |
| | | NSKNTLYLQMNSLRAEDTAVYYCAKVLGWFDYWG |
| | | QGTLVTVSSASTKGPSVFPLAPSSKSTSGGTAWLGCE |
| | | VTDYFPEPVTVSWNSGALTSGVHTFPAVLESSGLYSL |
| | | SSVVTVPSSSLGTQTYICNVNHKPSNTKVDKKVEPKS |
| | | CDGGGGSGGGGSQAVVTQEPSLTVSPGGTVTLTCGS |
| | | STGAVTTSNYANWVQEKPGQAFRGLIGGTNKRAPGT |
| | | PARFSGSLLGGKAALTLSGAQPEDEAEYYCALWYSN |
| | | LWVFGGGTKLTVLSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPEAAGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALGAPIEKTISKAKGQPREPQVYVLPPSREE |
| | | MTKNQVSLLCLVKGFYPSDIAVEWESNGQPENNYLT |
| | | WPPVLDSDGSFFLYSKLTVDKSRWQQGNVFSCSVMH |
| | | EALHNHYTQKSLSLSPG |
| 66 | Mab103 LC | EIVLTQSPGTLSLSPGERATLSCRASQSVSDEYLSWYQ |
| | | QKPGQAPRLLIHSASTRATGIPDRFSGSGSGTDFTLAIS |
| | | RLEPEDFAVYYCQQYGYPPDFTFGQGTKVEIKRTVA |
| | | APSVAIFPPSDERLKSGTASVVCVLNNFYPREAKVQW |
| | | KVDNALQSGNSQESVTEQDSKDSTYSLSSRLTLSKAD |
| | | YEKHKVYACEVTHQGLSSPVTKSFNRGEC |
| 67 | 83A10 CDR1L | RASQSVSSSYLAW |
| | (alternative to SEQ | |
| | ID NO: 18) | |
| 68 | 83A10 CDR2L | YGASSRAT |
| | (alternative to SEQ | |
| | ID NO: 19) | |
| 69 | Mab22 CDR1L | RASQSVSSYYLAW |
| | (alternative to SEQ | |
| | ID NO: 25) | |
| 70 | Mab22 CDR2L | SGAGSRAT |
| | (alternative to SEQ | |
| | ID NO: 26) | |
| 71 | Mab42 CDR1L | RASQSVSDEYLSW |
| | (alternative to SEQ | |
| | ID NO: 27) | |
| 72 | Mab42 CDR2L | HSASTRAT |
| | (alternative to SEQ | |
| | ID NO: 28) | |

Remark: SEQ ID NO: 20 and SEQ ID NO: 33 are identical

TABLE 6B
Antibody sequences (short list)

| | SEQ ID NO: | | | | | | | |
|---|---|---|---|---|---|---|---|---|
| CD3 | VH | VL | CDR1H | CDR2H | CDR3H | CDR1L | CDR2L | CDR3L |
| antibody | 7 | 8 | 1 | 2 | 3 | 4 | 5 | 6 |
| BCMA | VH | VL | CDR1H | CDR2H | CDR3H | CDR1L | CDR2L | CDR3L |
| antibody | | | | | | | | |
| 83A10 | 9 | 11 | 15 | 16 | 17 | 18 | 19 | 20 |
| Mab21 | 10 | 12 | 21 | 22 | 17 | 23 | 24 | 20 |
| Mab22 | 10 | 13 | 21 | 22 | 17 | 25 | 26 | 20 |
| Mab42 | 10 | 14 | 21 | 22 | 17 | 27 | 28 | 20 |
| Mab27 | 38 | 12 | 29 | 30 | 17 | 31 | 32 | 33 |
| Mab33 | 39 | 12 | 34 | 35 | 17 | 31 | 32 | 33 |
| Mab39 | 40 | 12 | 36 | 37 | 17 | 31 | 32 | 33 |

TABLE 7A
Additional constructs

| | SEQ ID NO: | | | |
|---|---|---|---|---|
| Fragment/Construct | 83A10 | Mab21 | Mab22 | Mab42 |
| BCMA CH1 | 41 | 41 | 41 | 41 |
| BCMA CL | 42 | 42 | 42 | 42 |
| CD3 CH1 | 43 | 43 | 43 | 43 |
| CD3 CL | 44 | 44 | 44 | 44 |

TABLE 7B
Additional constructs

| | SEQ ID NO: | | | |
|---|---|---|---|---|
| | 83 A10- | 21- | 22- | 42- |
| Construct | TCBcv | TCBcv | TCBcv | TCBcv |
| BCMA VH CH1cv × | 45 | 49 | 52 | 55 |
| CD3 VL_CH1 Fc knob | | | | |
| LALA PG (knob HC) | | | | |
| BCMAcv HC hole LALA PG | 46 | 50 | 53 | 56 |
| (hole HC) | | | | |
| BCMAcv hum IgG1 LC | 47 | 51 | 54 | 57 |
| (BCMAcv LC) | | | | |
| CD3 VH_CL (CD3 LC) | 48 | 48 | 48 | 48 |

TABLE 7C
Additional constructs

| | SEQ ID NO: | | |
|---|---|---|---|
| Construct | Mab101 | Mab102 | Mab103 |
| BCMA VH-CH1zw Fc_zwA (HC-1) | 58 | 61 | 64 |
| BCMA VH-CH1zw × CD3 VL-CH1 | 59 | 62 | 65 |
| Fc_zwB (HC-2) | | | |
| BCMAzw hum IgG1 LC (BCMA LC) | 60 | 63 | 66 |
| CD3 VH_CL (CD3 LC) | 48 | 48 | 48 |

### EXAMPLES

### Example 1: BCMA Surface Expression on Plasmablasts and Plasma Cells and Soluble BCMA Levels in Samples from Normal Healthy Volunteers (NHV) and AAV Patients

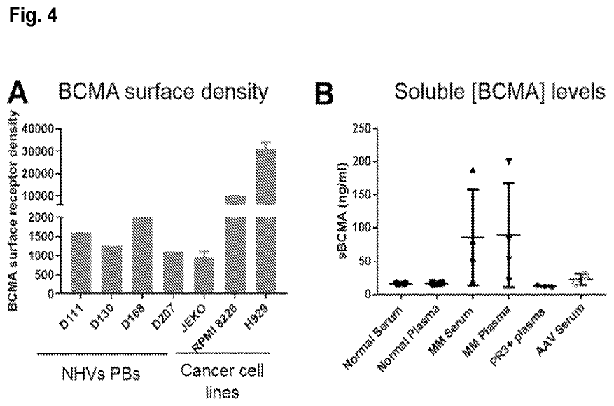

Peripheral blood mononuclear cells (PBMC) were isolated from whole blood collected from four normal healthy volunteers (NHV) using Ficoll gradient. BMCA expression was assessed on plasmablasts (PB) by flow cytometry. Plasmablasts were identified as CD19(+) CD20(−) CD27(+) CD38(+). Anti-BCMA antibody coated fluorescent beads were used to generate standard curves to compare mean fluorescent intensity to BCMA surface receptor density. BCMA-expressing cancer cell lines (JEKO, RPMI-8226 and H929) were profiled for comparison (FIG. 4A). BCMA surface expression is much lower on plasmablasts derived from NHVs than on the multiple myeloma cell lines RPMI-8226 and H929 for all tested NHVs.

Soluble BCMA levels were assessed by ELISA in serum or plasma samples from NHV (‘Normal’), Multiple Myeloma (‘MM’) or ANCA-Associated Vasculitis (‘AAV’) patients (FIG. 4B). Soluble BCMA levels are much lower in NHV samples than in MM samples, reflecting the lower levels of BCMA cell surface expression on the plasmablasts in NHV as compared to MM patients. Soluble BCMA levels in both serum and plasma (PR3+) samples from AAV is comparable to NHV samples, and much lower than in MM samples. In view of this correlation, BCMA surface expression on plasmablasts and plasma cells in AAV is anticipated to be comparable to that in NHV.

### Example 2: Generation of T Cell Bispecific Antibodies

Anti-BCMA anti-CD3 bispecific antibodies were generated having the format 1^{st} BCMA Fab-Fc-CD3 Fab-2^{nd} BCMA Fab (referred to herein as “2+1” format). Methods of making Anti-BCMA anti-CD3 bispecific antibodies can be found in WO2017/021450, which is incorporated herein by reference.

HD1 denotes that:
the CH3 domain of the Fc comprises one chain with the amino acid substitution T366W and one chain with the amino acid substitutions T366S, L368A, and Y407V (as set forth in Table 2); and
both of the BCMA Fabs comprise the amino acid substitutions K147E and K213E in the CH1 domain and the amino acid substitutions E123R and Q124K in the CL domain (as set forth in Table 4); HD2 denotes that:
the CH2-CH3 domains of the Fc comprises one chain with the amino acid substitutions T350V, L351Y, F405A, Y407V and one chain with the amino acid substitutions T350V, T366L, K392L, T394W (as set forth in Table 3);
both BCMA Fabs comprise the amino acid substitutions A141W, L145E, K147T, Q175E in the CH1 domain and the amino acid substitutions F116A, Q124R, L135V, T178R in the CL domain (as set forth in Table 5)
(heavy chain constant region amino acid positions numbered according to EU numbering; light chain constant region amino acid positions numbered according to Kabat).

In addition to the above modifications noted under HD1 and HD2, the Fc of the bispecific antibodies in the present Examples contains the amino acid substitutions P329G, L234A and L235A (positions numbered according to EU numbering).

The bispecific antibodies in the present Examples also comprise “CrossMAb” technology in which the VH and VL of the CD3 Fab were exchanged.

A summary of the antibodies generated in the present Examples is provided in Table 8.

TABLE 8
Anti-BCMA anti-CD3 bispecific antibodies generated in the
present Examples.

| Bispecific | |
|---|---|
| antibody name | Structure |
| CC-93269 | 2 + 1 (BCMA SEQ ID NO: 10 + 14) |
| | (CD3 SEQ ID NO: 7 + 8)HD1 |
| Mab101 | 2 + 1 (BCMA SEQ ID NO: 9 + 11) |
| | (CD3 SEQ ID NO: 7 + 8)HD2 |
| Mab102 | 2 + 1 (BCMA SEQ ID NO: 1 0+ 13) |
| | (CD3 SEQ ID NO: 7 + 8)HD2 |

### Example 3: Dose-Dependent BCMA T Cell Engager Killing of BCMA-Expressing Cells Occurs with T-Cell Activation

JEKO Cells

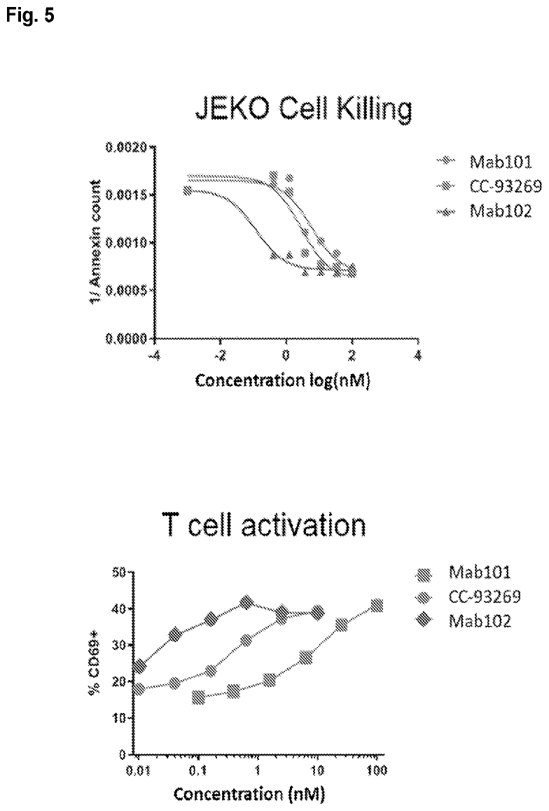

JEKO cells were cultured with CD3+ T cells (rested overnight) at a 1:2 target:effector (T:E) ratio and various concentrations of anti-BCMA anti-CD3 bispecific antibodies (BCMA T cell engagers). T-cell mediated killing of JEKO cells was assessed by annexin V expression measured over 24 hours, with images recorded every two hours. Annexin V+ cell counts were determined using the Incucyte ZOOM software. Data and EC50 values calculated at the 20 hour time point is shown in FIG. 5A and Table 9.

TABLE 9
EC50 for killing of JEKO cells with BCMA T cell engagers

| | Mab101 | CC-93269 | Mab102 |
|---|---|---|---|
| EC50 for JEKO cell killing | 5.724 | 2.791 | 0.1196 |
| (nM) | | | |

This data shows that the BCMA T cell engagers (BCMA TCE) are capable of killing cells expressing BCMA at levels comparable to that on plasmablasts from normal healthy volunteers.

T cell activation was analyzed by flow cytometry at the 24 hour time point. Cells were washed and then stained for T cell lineage markers (CD3, CD4 and CD8) and activation markers (CD69, CD25, and CD154). Flow cytometry samples were acquired on a BD LSRFortessa and analyzed using Treestar FlowJo X software. Cell imaging performed on Incucyte Live Cell Analysis Imaging System. Data plotted and EC50 values calculated using Graphpad Prism 7 software.

FIG. 5B shows T cell activation as represented by CD69 expression on CD8+ T cells.

These data show an elevation in the frequency of activated T cells at the 50% effective concentration (EC50) of BCMA T cell engager for killing of JEKO cells.

MM Cells

The Multiple Myeloma cell line RPMI-8226 was co-cultured with NHV PBMCs at different target:effector (T:E) ratios and various concentrations of CC-93269. T-cell mediated killing of RPMI-8226 cells was assessed by annexin V expression measured over 96 hours, with images recorded every hour. Annexin V+ cell counts were determined using the Incucyte ZOOM software; the 25 hour and 72 hour time points are shown in FIG. 6A.

T-cell activation was analysed by analyzed by flow cytometry at the 25 hour and 72 hour time points. After 25 hours, cells were washed and then stained for CD8 T cell lineage and CD69 expression as a marker of T-cell activation (FIG. 6B).

These data show that significant T-cell activation occurs at doses of CC-93269 equal to or less than the doses for MM cell killing.

### Example 4: Dose-Dependent BCMA T Cell Engager Killing of Plasmablasts from Healthy Volunteers Occurs with Minimal T-Cell Activation

Peripheral blood mononuclear cells (PBMC) were isolated from whole blood collected from healthy volunteers using Ficoll gradient resuspended in RPMI+10% HI FBS. PBMCs were treated with various concentrations of BCMA TCE or control 2+1 anti-HEL anti-CD3 antibody. Following 24 hour incubation, plasmablast killing, T cell activation and cytokine production were assessed.

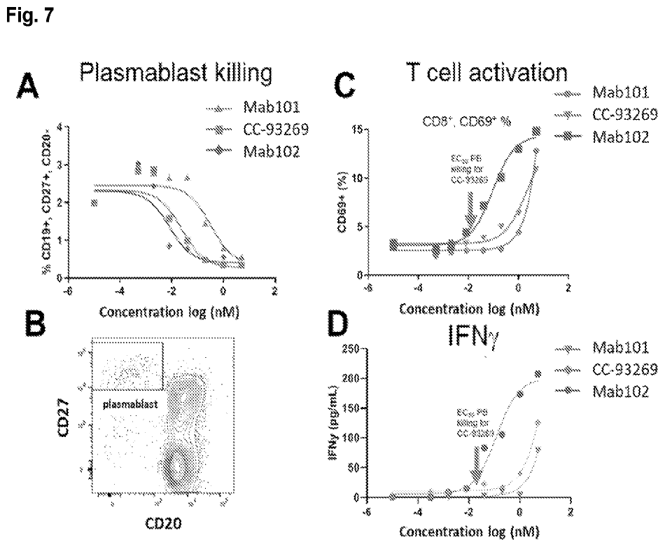

Plasmablast killing was assessed by FACS whereby plasmablasts are identified as CD19(+) CD20(−) CD27(+) cells and given as percent of total CD19(+) cells normalized to the untreated control. CD19(+) CD20(−) CD27(+) cells were confirmed to have the additional known markers of plasmablasts: BCMA(+) SLAMF7(+) IgD(−) CD38(+) CD138(−). FIG. 7A is a representative dose-response curve and FIG. 7B is a representative FACS plot gated on CD3(−) CD19(+) cells. The 50% effective concentration (EC50) of CC-93269 for plasmablast killing in healthy volunteers (n=12) is 0.005 nM. Plasmablast killing occurs at concentrations of BCMA TCE lower than that needed to kill BCMA-expressing cancer cell lines (e.g. JEKO cells).

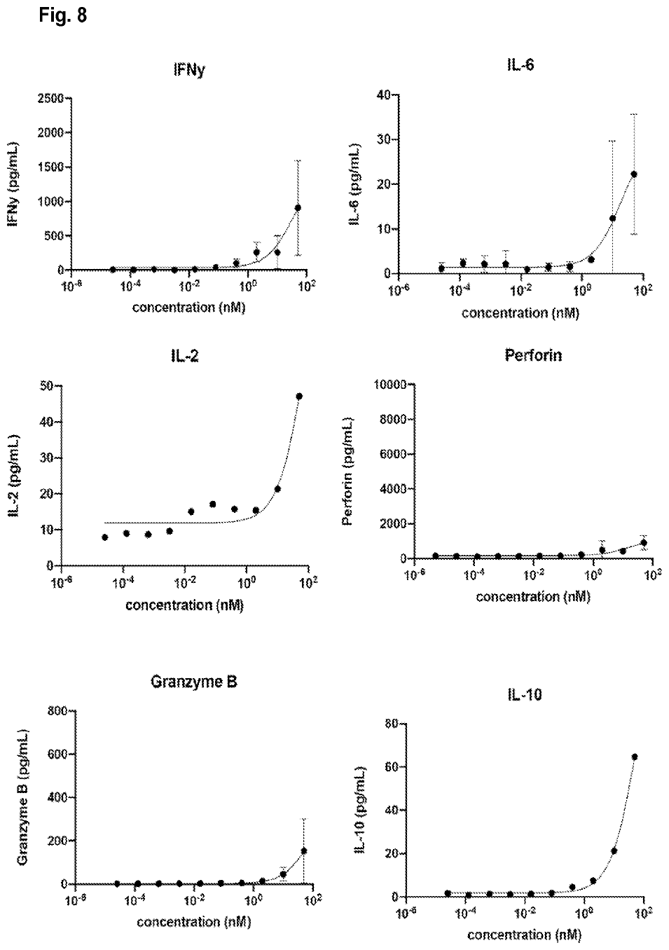

For T cell activation, cells were washed and then stained for T cell lineage (CD3, CD4 and CD8) and activation markers (CD69, CD25, and CD154). Data represented in FIG. 7C is CD69 expression on CD8(+) T cells. Culture supernatants were analyzed for cytokine production (IFNγ, IL-6, IL-2, IL-10, granzyme B and perforin) using the MSD Pro-inflammatory I assay (FIG. 7D, FIG. 8). The data in FIG. 8 are for CC-93269.

Together, these data show minimal elevation in the frequency of activated T cells (i.e. less than 20% above the baseline) and minimal cytokine production (i.e. less than 20 pg/mL above the baseline) at the 50% effective concentration (EC50) of BCMA TCE for killing of plasmablasts from PBMC of healthy volunteers.

Table 10 summarizes data for CC-93269, and illustrates minimal elevation in the frequency of activated T cells at the 90% effective concentration (EC90) for depletion of plasmablasts. CC-93269 shows deep depletion of plasmablasts (>90%) in PBMC from healthy volunteers in vitro, in the absence of significant T cell activation.

TABLE 10
Plasmablast killing and T cell activation in PBMC from healthy
volunteers with CC-93269

| | | | CD69+ CD8+ T cells at: | |
|---|---|---|---|---|
| | Plasmablast depletion | | EC₉₀ PB | EC₉₉ PB |
| | EC₉₀ (nM) | EC₉₉ (nM) | depletion | depletion |
| CC-93269 | 0.054 | 0.594 | Baseline | Modest |
| (n = 11) | | | | (< 5% CD8+ cells) |

### Example 5: Effect on Other B Cell Populations in PBMCs from Healthy Volunteers after Culture with CC-93269

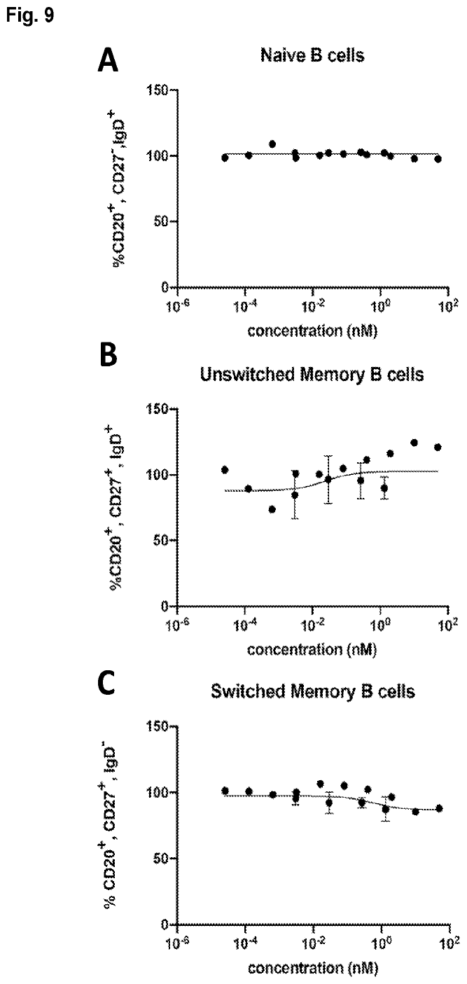

CC-93269-treated PBMC samples from Example 4 were stained for B cell lineage markers (CD20, CD27, and IgD). Memory B cells were identified as cells displaying the markers CD19 (+) CD20 (+) CD27 (+), and then further confirmed with the markers IgD (−) CD38 (−) BCMA (+/−). Data is represented in FIG. 9A-C which is given as a percent of total CD19(+) CD20(+) cells. This data shows that CC-93269 does not significantly deplete naïve, unswitched or switched memory B cell populations in PBMCs from healthy volunteers in vitro at the 90% effective concentration (EC90) for plasmablast killing.

### Example 6: Dose-Dependent BCMA TCE Mediated Killing of Plasmablasts in Manipulated Bone Marrow with Minimal T-Cell Activation

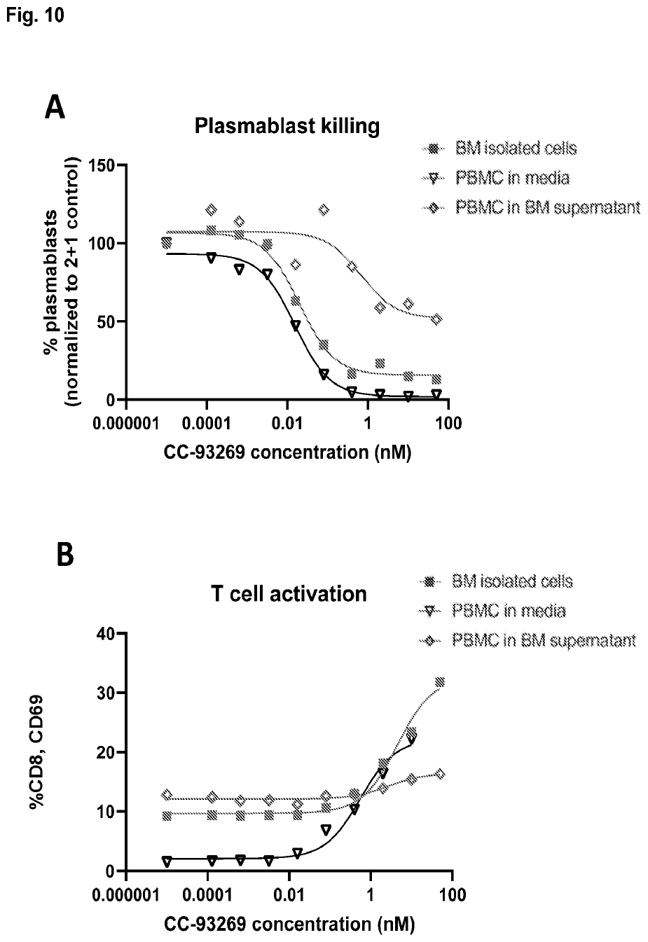

Bone marrow (BM) mononuclear cells were isolated from the bone marrow of healthy volunteers using Ficoll gradient and then treated with various concentrations of BCMA TCE or control 2+1 anti-HEL anti-CD3 antibody. Following 24 hours incubation, plasmablast killing (FIG. 10A, Table 11) and T cell activation (FIG. 10B) were assessed by flow cytometry, as in Example 4. PBMC isolated from healthy volunteers were suspended either in media or in bone marrow (BM) supernatant and then treated with BCMA TCE or control 2+1 anti-HEL anti-CD3 antibody for 24 hours for comparison.

TABLE 11
Plasmablast killing in bone marrow mononuclear cells with
BCMA TCE

| | Mab101 | CC-93269 | Mab102 |
|---|---|---|---|
| EC50 for BM plasmablast | 0.2662 | 0.02012 | 0.002807 |
| killing (nM) | | | |

These data show that BCMA TCEs induce killing of plasmablasts from bone marrow at similar concentrations to plasmablasts from PBMC suspended in media, and with minimal T-cell activation. This data is significant as long-lived plasmablasts and plasma cells are preferentially found in the bone marrow.

### Example 7: Dose-Dependent BCMA TCE Mediated Killing of Plasmablasts from AAV with Minimal T-Cell Activation

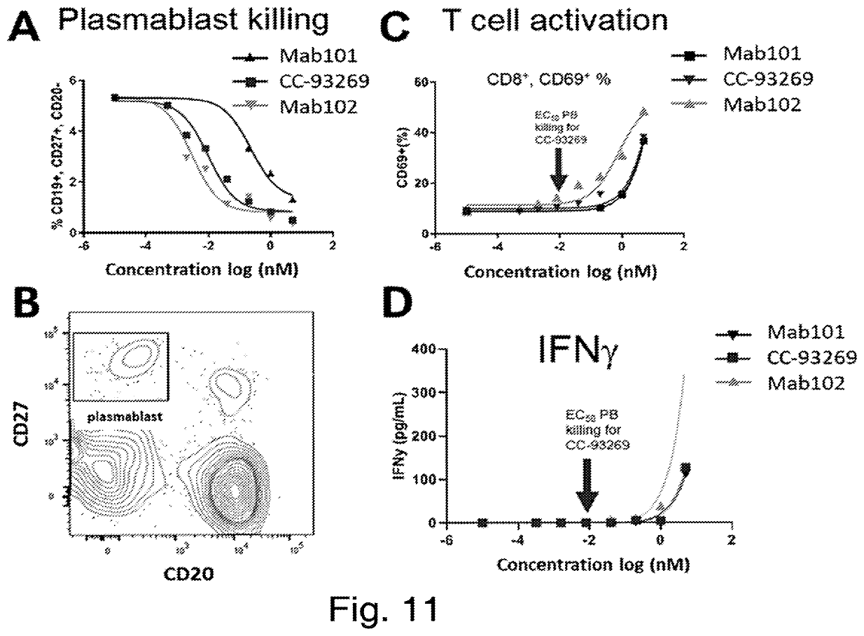

Peripheral blood mononuclear cells (PBMC) were isolated from whole blood collected from AAV patients, including AAV relapsed or refractory to rituximab, methotrexate and folic acid, using Ficoll gradient and were then treated with various concentrations of BCMA TCE (CC-93269, Mab101 or Mab102) or control 2+1 anti-HEL anti-CD3 antibody. Following 24 hour incubation, plasmablast killing (FIG. 11A-11B), T cell activation (FIG. 11C, 12A-12C) and cytokine production (FIG. 11D) were assessed.

Plasmablast killing was assessed by FACS whereby plasmablasts are identified as CD19(+) CD20(−) CD27(+) cells, and are given as percent of total CD19(+) cells normalized to the untreated control (FIG. 11A). CD19(+) CD20(−) CD27(+) cells were confirmed to have the additional known markers of plasmablasts: BCMA(+) SLAMF7(+) IgD(−) CD38(+) CD138(−). FIG. 11B is a representative FACS plot gated on CD3(−) CD19(+) cells. The 50% effective concentration (EC50) of CC-93269 for plasmablast killing in AAV is 0.007 nM. AAV plasmablast killing occurs at concentrations of the BCMA TCE lower than that needed to kill the JEKO cancer cell lines despite the similar levels of BCMA expression.

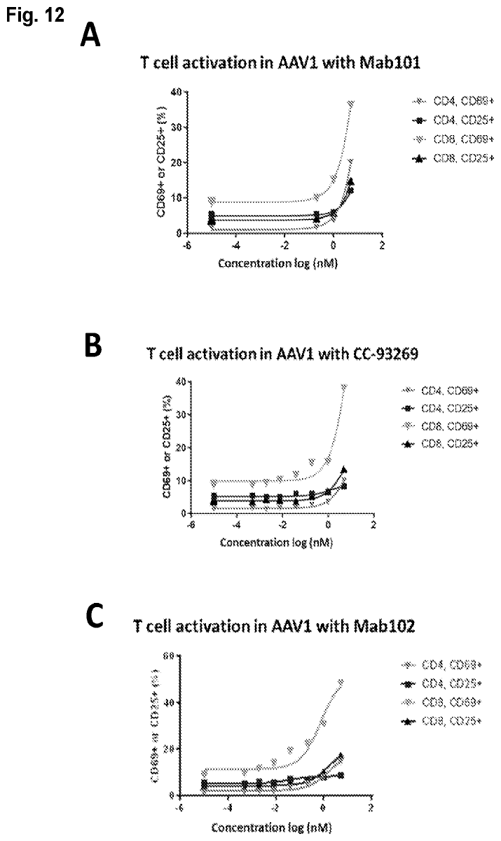

For T cell activation, cells were washed and then stained for T cell lineage (CD3, CD4 and CD8) and activation markers (CD69, CD25, and CD154). Data represented in FIG. 11C is CD69 expression on CD8(+) T cells. Data represented in FIG. 12A-C is CD69 or CD25 expression levels on CD4(+) T cells or CD8(+) T cells.

Culture supernatants were analyzed for cytokine production (IFNγ, IL-6, TNFα, IL-1β, granzyme A, granzyme B and perforin) using the MSD Pro-inflammatory I assay. The data for IFNγ are shown in FIG. 11D.

Together, these data show no considerable elevation in the frequency of activated T cells (i.e. less than 20% above the baseline), nor in cytokine production (i.e. less than 20 pg/mL above the baseline), at AAV plasmablast killing-competent doses of BCMA TCEs.

Table 12 summarizes the data from Examples 4 and 7, highlighting a window of BCMA TCE concentration for plasmablast (PB) killing without T cell activation. In this window, adverse events linked to the activation of T cells or excessive cytokine production, such as cytokine release syndrome (CRS), are less likely to occur.

TABLE 12
The therapeutic window for BCMA TCEs to achieve plasmablast
(PB) killing without T cell activation

| | | | EC₅₀ T cell | | Window |
|---|---|---|---|---|---|
| | | | activation | | between |
| | | EC₅₀ | (nM) | | killing |
| | | PB | CD8, | IFNg | and |
| TCE | | killing | CD69+ | secretion | T cell |
| treatment | Donor | (nM) | (nM) | (nM) | activation |
| Mab101 | Healthy | 0.257 +/− | >1 | >1 | ≥3 |
| (Kd | volunteers | 0.087 | | | |
| BCMA = | (n = 2) | | | | |
| 1.1 nM) | AAV-1 | 0.236 | >1 | >1 | ≥4 |
| | (n = 1) | | | | |
| CC-93269 | Healthy | 0.029 +/− | ≥0.68 +/− | ≥0.92 +/− | 16-22 |
| (Kd | volunteers | 0.029 | 0.04 | 0.12 | |
| BCMA = | (n = 6) | | | | |
| 0.14 nM) | AAV-1 | 0.010 | >1 | >1 | >100 |
| | (n = 1) | | | | |
| Mab102 | Healthy | 0.007 +/− | 0.63 +/− | 0.91 +/− | 90- |
| (Kd | volunteers | 0.006 | 0.61 | 1.1 | >100 |
| BCMA = | (n = 3) | | | | |
| 0.06 nM) | AAV-1 | 0.003 | 0.77 | >1 | >100 |
| | (n = 1) | | | | |

### Example 8: Dose-Dependent CC-93269 Mediated Killing of Plasmablasts from AAV Patient Treated with rituximab

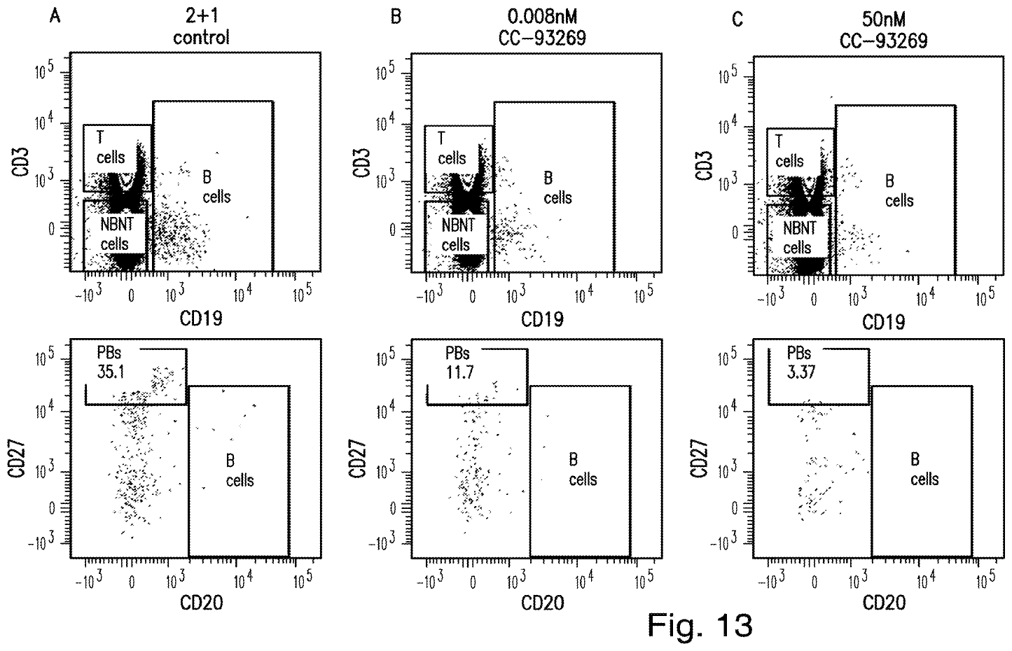

Peripheral blood mononuclear cells (PBMC) were isolated from whole blood of an AAV patient, AAV-5, who had last received rituximab 5 months prior, by Ficoll gradient and resuspended in RPMI+10% HI FBS. PBMCs were treated with increasing concentrations of CC-93269 (FIG. 13B-13C) or control 2+1 anti-HEL anti-CD3 antibody (FIG. 13A) for 24 hours, then assessed by flow cytometry. Cells were gated for viability and singlets to reach the live cell FACS plots (FIG. 13). Plasmablasts are defined as CD19+CD27+CD20−. Plasmablasts were also confirmed to be CD38+, BCMA+, and CD138− (data not shown). From the CD19+CD20+ gate, naïve B cells are defined as CD27−IgD+, unswitched memory B cells as CD27+ IgD+ and switched memory B cells as CD27+ and IgD−. Representative dot plots from a normal healthy volunteer are shown. A lack of CD20 (+) B cells but a high CD20(−) CD27(+) plasmablast count was observed in the control (FIG. 13A), showing that rituximab results in B cell depletion without plasmablast depletion. CC-93269 induced selective depletion of plasmablasts with minimal B cell depletion at a sub-nanomolar concentration (FIG. 13B). CC-93269 rapidly depletes plasmablasts from PBMCs from AAV patients, even when the patients have been treated with immunosuppressants e.g. rituximab.

### Example 9: BCMA-TCE in Absence of Target does not Lead to Elevated Frequency of Activated T Cells in AAV Patient PBMCs

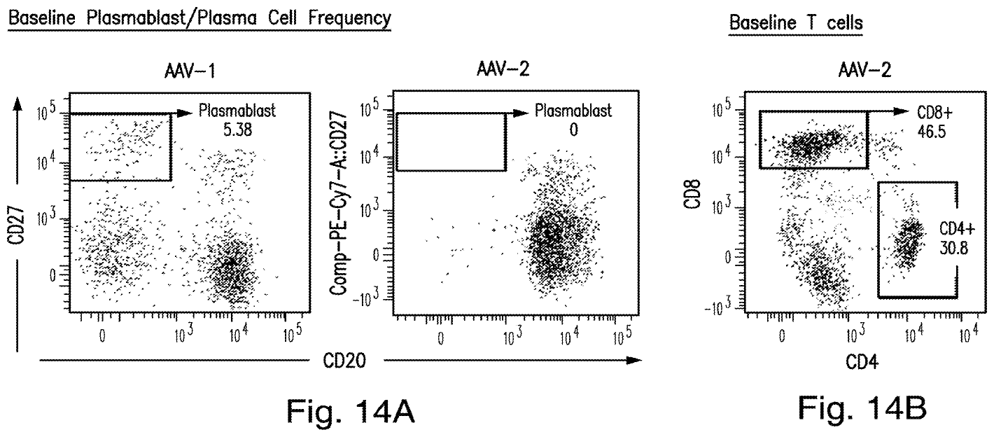

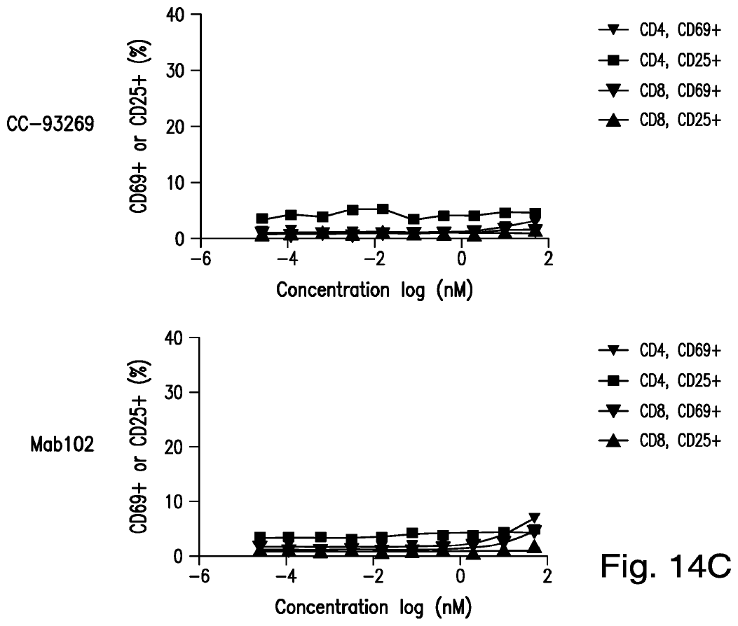

PBMCs were isolated from an AAV patient, AAV-2, who had previously been treated with rituximab, mycophenolate, dexamethasone and methylprednisolone. FIG. 14A illustrates FACS plots showing a lack of CD19(+) CD20(−) CD27(+) plasmablast and plasma cell targets in AAV-2 subject at baseline compared to AAV-1 subject. Plots are gated on CD3(−) CD19(+) cells. FIG. 14B illustrates a FACS plot showing an adequate presence of CD4(+) and CD8(+) T cells in AAV-2 subject. Plot is gated on CD3(+) cells.

PBMCs from AAV-2 were treated with various concentrations of BCMA TCE and following 24 hour incubation, T cell activation was assessed by flow cytometry as in Example 7. FIG. 14C illustrates the frequency of CD69(+) or CD25(+) on CD4(+) or CD8(+) T cells. These data show BCMA-TCE does not lead to T cell activation when target plasmablasts/plasma cells are absent.

### Example 10: T-Cell Activation is Lower when BCMA-Expressing Cancer Cells are Killed at an Effector: Target Ratio Similar to AAV

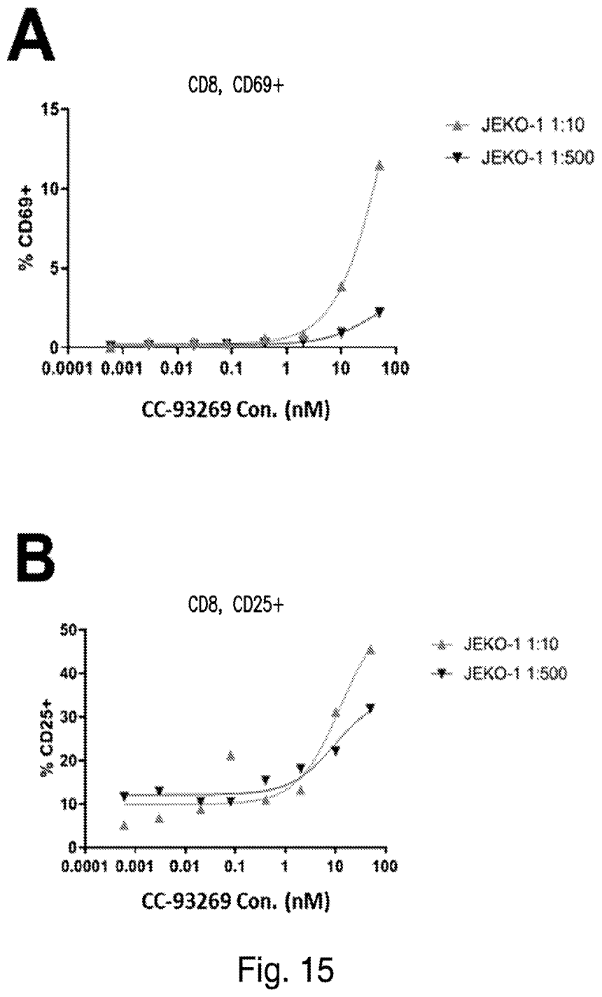

JEKO-1 cells (2500 cells/well) were cultured with PBMC at target:effector (T:E) ratios of 1:10 or 1:500 to mimic T:E ratios (BCMA+ cells: T cells) observed in Multiple Myeloma (MM) or AAV, respectively. Following 24 hour incubation with CC-93269 or control 2+1 anti-HEL anti-CD3 antibody, cells were washed and then CD69 (FIG. 15A) and CD25 (FIG. 15B) expression on CD8(+) T cells were assessed.

These data indicate that greater T:E ratios (BCMA+ cells: T cells) result in a greater degree of T cell activation. Notably, the frequency of activated T cells is lower when the T:E ratio is comparable to the ratio of BCMA-expressing plasmablasts and T cells found in healthy volunteers and AAV patients, than when the T:E ratio mimics MM patients.

### Example 11: BCMA-TCE Abrogates the Ability of IgG-Producing Plasmablasts and Plasma Cells to be Regenerated Despite Appropriate Plasmablast/Plasma Cell Growth Factor Stimulus

Peripheral blood mononuclear cells (PBMC) were isolated from whole blood collected from healthy volunteers using Ficoll gradient and treated with various concentrations of BCMA TCE (CC-93269, Mab101 or Mab102) or control 2+1 anti-HEL anti-CD3 antibody. Patient D214 was treated with BCMA TCE at the 90% effective concentration (EC90) for depletion of plasmablasts.

Following 24-hour incubation, PBMC were cultured with growth factors IL-2 (20 U/ml) BAFF (200 ng/ml) and IL-21 (100 ng/ml) for 4-7 days to induce plasmablast/plasma cell differentiation from BCMA negative precursors. Some cultures were also stimulated with CpG (ODN200610 μg/mL) for this period.

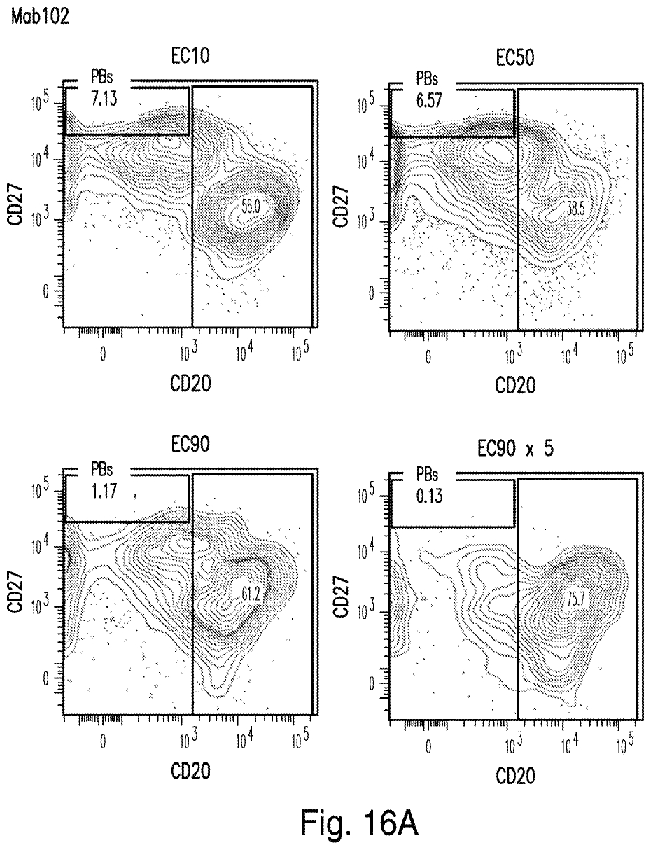

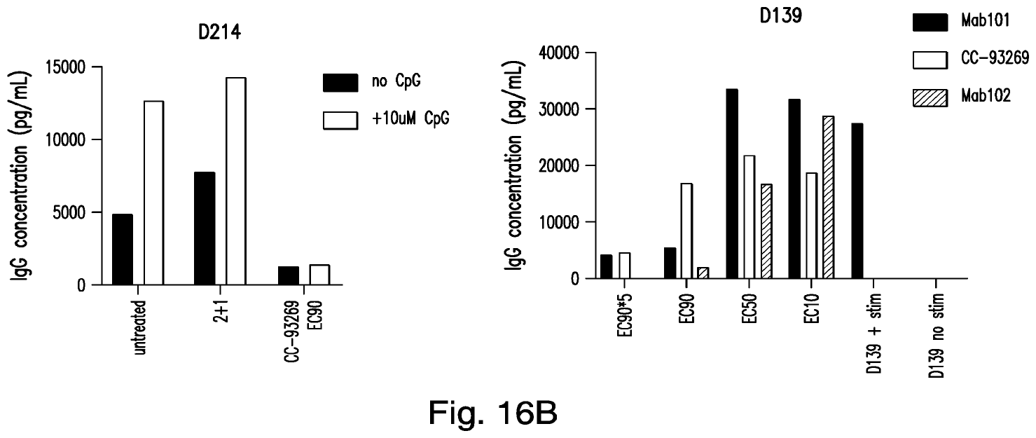

Following culture, plasmablasts and plasma cells (CD19(+) CD20(−) CD27(+)), or CD20⁺ B cells, were measured by flow cytometry (FIG. 16A). BCMA-TCEs suppress the recovery of plasmablasts and plasma cells after depletion despite appropriate growth factors for their re-generation, particularly at the 90% effective concentration (EC90) for plasmablast killing.

Culture supernatants were collected to measure total IgG secretion by ELISA (FIG. 16B). BCMA-TCE suppresses the production of IgG antibodies despite stimulation with CpG particularly at the EC90 concentration for plasmablast killing. This suggests that IgG autoantibody production can be suppressed by depleting plasmablasts in vivo by 90%.

### Example 12: Dose-Dependent BCMA TCE Mediated Killing of Plasmablasts from Rheumatoid Arthritis with Minimal T-Cell Activation

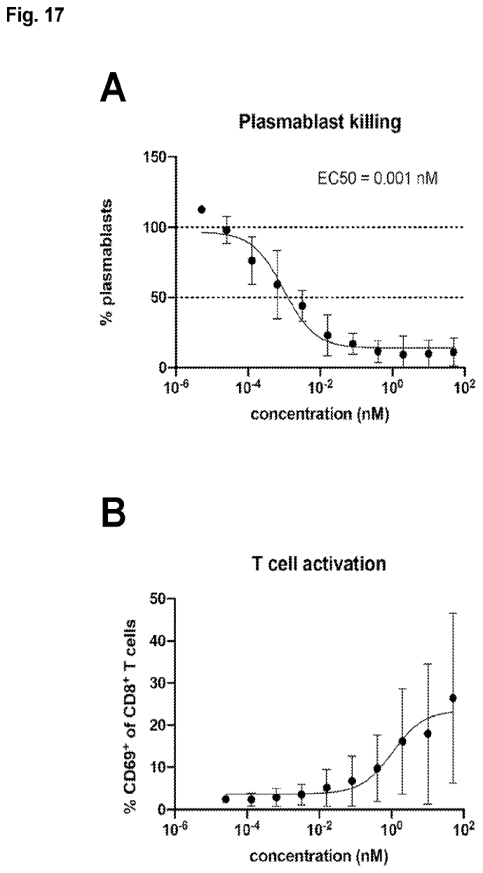

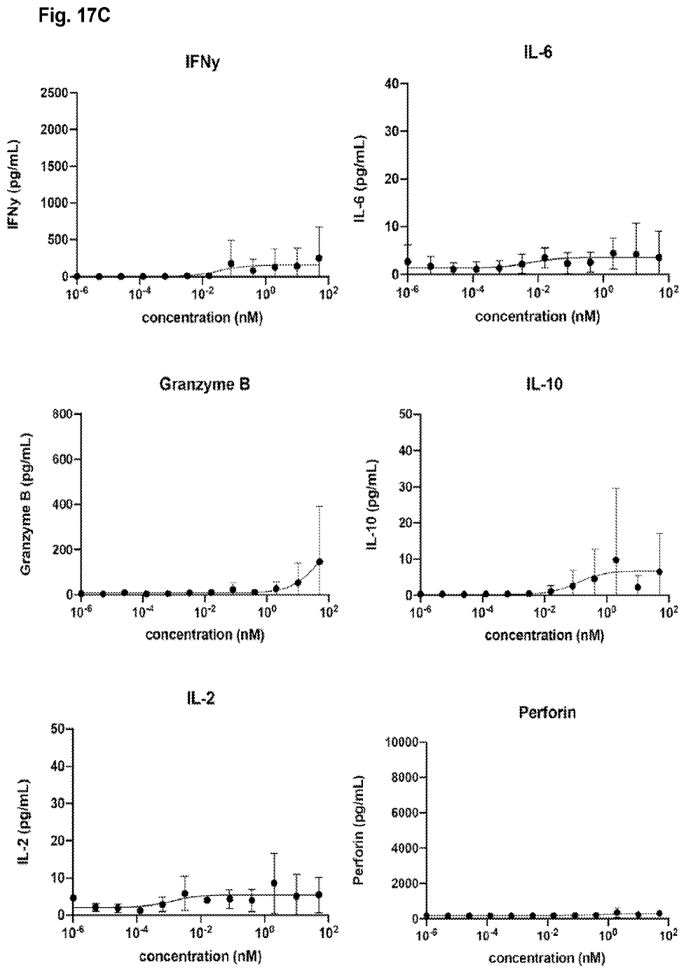

PBMCs were isolated from Rheumatoid Arthritis (RA) patients and treated with various concentrations of CC-93269. Following 24 hour incubation, plasmablast killing (FIG. 17A), T cell activation (FIG. 17B), and cytokine secretion (FIG. 17C) were assessed by flow cytometry as in Example 4.

The 50% effective concentration (EC50) of CC-93269 for plasmablast killing in RA is 0.001 nM. Therefore, RA plasmablast killing occurs at concentrations of BCMA TCE lower than required for T cell activation or cytokine secretion.

### Example 13: Effect on Other B Cell Populations in PBMCs from RA Patients after Culture with CC-93269

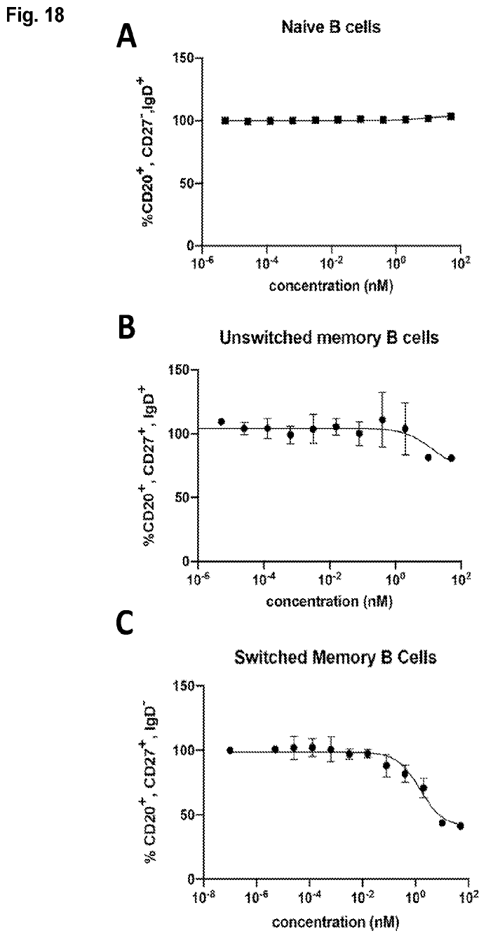

CC-93269-treated PBMC samples from Example 12 were stained for B cell lineage markers (CD20, CD27, and IgD). Data represented in FIG. 18A-C is given as a percent of total CD19(+)CD20(+) cells. The data shows that CC-93269 does not significantly deplete naïve, unswitched or switched memory B cell populations in PBMCs from RA patients in vitro at EC90 concentrations for plasmablast killing.

### Example 14: Dose-Dependent BCMA TCE Mediated Killing of Plasmablasts from Systemic Lupus Erythematosus Patients Occurs with Minimal T-Cell Activation

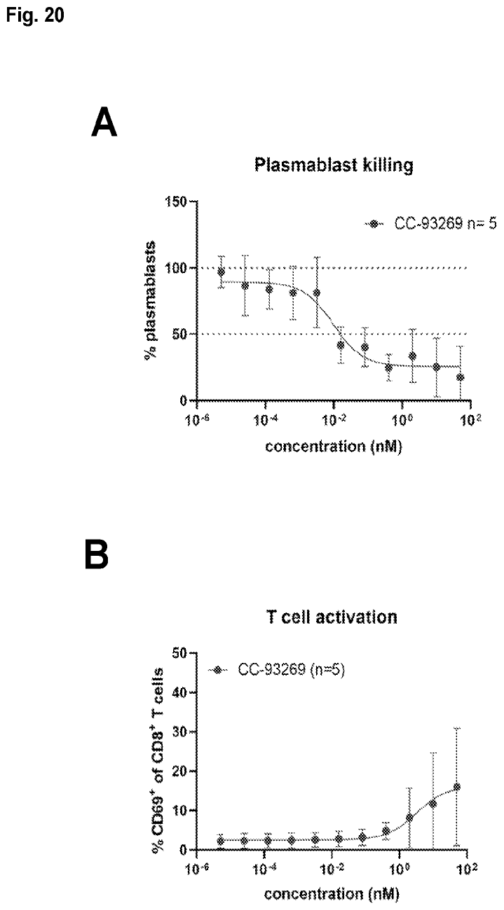

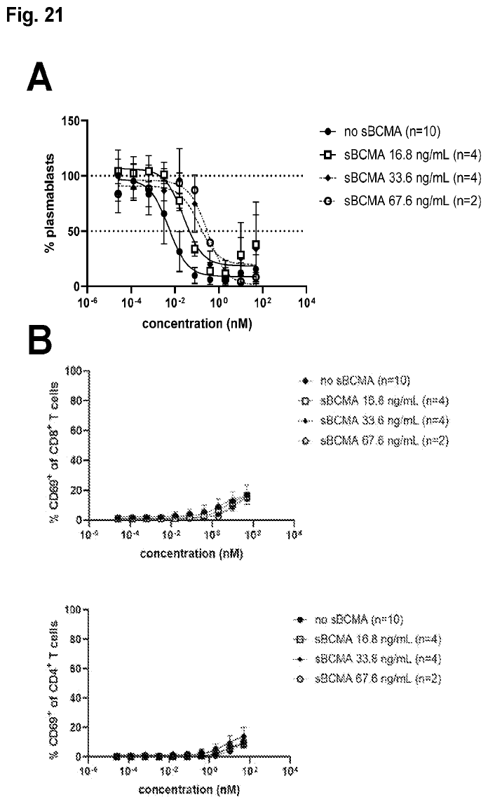

PBMCs were isolated from systemic lupus erythematosus (SLE) patients and treated with various concentrations of CC-93269 or control 2+1 antibody. Following 24 hour incubation, plasmablast killing (FIG. 20A) and T cell activation (FIG. 20B) were assessed as in Example 4.

The 50% effective concentration (EC50) of CC-93269 for plasmablast killing in SLE is 0.01 nM (n=5). Therefore, SLE plasmablast killing occurs at concentrations of BCMA TCE lower than required for T cell activation.

### Example 15: Selective Depletion of Plasmablasts by CC-93269 in Cynomolgus Macaque

PBMCs were isolated from whole blood of cynomolgus macaque. Plasmablast and CD20(+) B cells were treated with various concentrations of CC-93269 or control 2+1 anti-HEL anti-CD3 antibody for 24 hours.

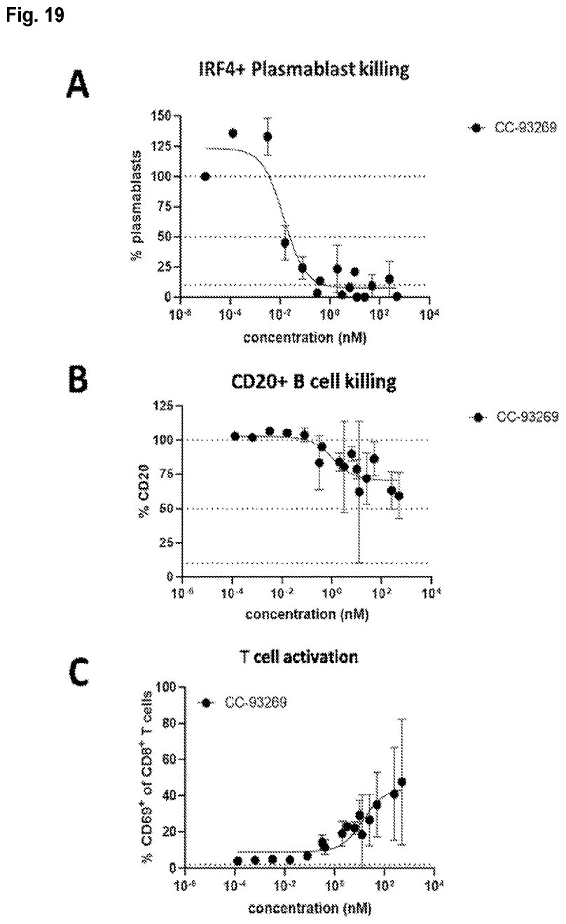

Plasmablast killing was assessed by FACS whereby plasmablasts are identified as CD19(+)IRF4(+), and given as a percent of total CD19(+) cells (FIG. 19A) CD20(+) B cell killing was also assessed by FACS whereby CD19(+)CD20(+) cells are given as a percent of total CD19(+) cells (FIG. 19B). IRF4+ plasmablast killing occurs at a lower concentration of CC-93269 than that needed to kill CD20(+) B cells. Thus, CC-93269 is capable of selectively depleting IRF4+ plasmablasts, without broad CD20 (+) B cell depletion.

T cell activation was assessed by FACS whereby CD69(+)CD8(+) T cells are given as a percent of total CD8(+) T cells (FIG. 19C).

Together, these data show selective killing of plasmablasts by CC-93269 and minimal elevation in the frequency of activated T cells at concentrations of CC-93269 for IRF4+ plasmablast depletion without CD20 (+) B cell depletion in an accepted pharmacokinetic-pharmacodynamic (PK/PD) model. They can be used to predict the pharmacological and toxicological effects of BCMA-TCE in vivo.

### Example 16: Effect of Exogenous Soluble BCMA on CC-93269-Mediated Killing of Plasmablasts from Healthy Volunteers

PBMCs were isolated from normal healthy volunteers and various concentrations of exogenous soluble BCMA (sBCMA) was added. The maximum concentration of 67.6 ng/mL sBCMA was chosen as it represents twice the upper limit of sBCMA levels in autoimmune patients (data not shown). Soluble BCMA levels in serum or plasma from donor patients having an autoimmune disorder was assessed by bead-based immunoassay by Ampersand Biosciences (Lake Clear, NY).

The samples were then treated with increasing concentrations (0-50 nM) of CC-93269 or control 2+1 antibody for 24 hours before plasmablasts killing (FIG. 21A) and T cell activation (FIG. 21B) were assessed.

Plasmablast killing was assessed by FACS whereby plasmablasts are identified as CD19(+) CD20(−) CD27(+) cells and given as percent of total CD19(+) cells normalized to the untreated control (FIG. 21A). Plasmablast killing in the presence of sBCMA occurs at concentrations of CC-93269 lower than that needed to kill BCMA-expressing cancer cell lines (e.g. JEKO cells), even in the concentrations of sBCMA that would be present in autoimmune patients, and higher.

For T cell activation, cells were washed and then stained for T cell lineage (CD4 and CD8) and activation markers (CD69, CD25). Data represented in FIG. 21B illustrates the frequency of CD69(+) on CD4(+) T cells or on CD8(+) T cells.

Table 13 summarizes data for CC-93269, and illustrates minimal elevation (i.e. less than 20% above the baseline) in the frequency of activated T cells at increasing sBCMA levels at the 90% effective concentration (EC90) of BCMA TCE for in vitro depletion of plasmablasts in PBMC from healthy volunteers.

TABLE 13
Effect of exogenous soluble BCMA on plasmablast killing and T
cell activation in PBMC from healthy volunteers with CC-93269

| Soluble | | | CD69+ CD8+ T cells at: | |
|---|---|---|---|---|
| BCMA | Plasmablast depletion | | EC₅₀ PB | EC₉₀ PB |
| (ng/ml) | EC₅₀ (nM) | EC₉₀ (nM) | depletion | depletion |
| 0 | 0.005 | 0.047 | < 20% | < 20% |
| 16.8 | 0.025 | 0.225 | < 20% | < 20% |
| 33.6 | 0.130 | 1.17 | < 20% | < 20% |
| 67.6 | 0.32 | 2.88 | < 20% | < 20% |

### Example 17: Minimal CC-93269 Mediated T-Cell Activation and Cytokine Secretion in Whole Blood Samples from Healthy Volunteers and AAV Patients

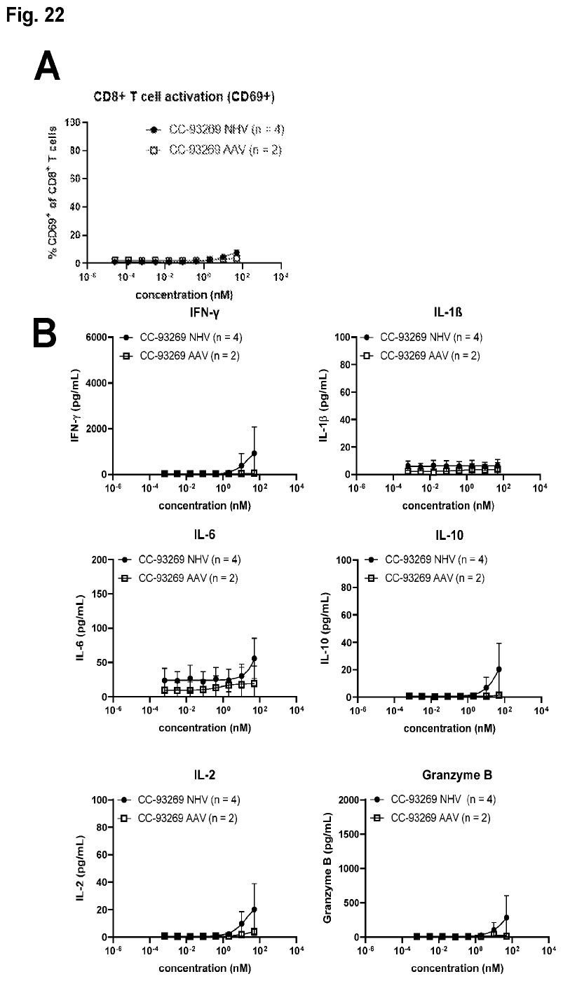

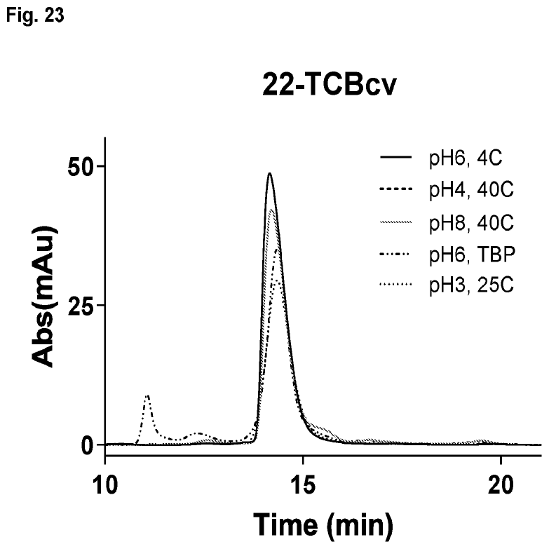

Whole blood samples from normal healthy volunteers (n=4) and AAV patients (n=2) were treated with various concentrations of CC-93269 or control 2+1 antibody. Following 24 hour incubation, T cell activation was assessed as in Example 4. Data represented in FIG. 22A is CD69 expression on CD8(+) T cells in whole blood samples from normal healthy volunteers (NHV) and AAV patients.

Culture supernatants were analyzed for cytokine production (IFNγ, IL-1β, IL-6, IL-2, IL-10, and granzyme B) using the MSD Pro-inflammatory I assay (FIG. 22B).

Together, these data show minimal elevation (i.e. less than 20% above the baseline) in the frequency of activated T cells and minimal cytokine production (i.e. less than 20 pg/mL above the baseline) when whole blood samples from normal healthy volunteers are treated with CC-93269.

### Example 18: Antibodies with Improved Stability

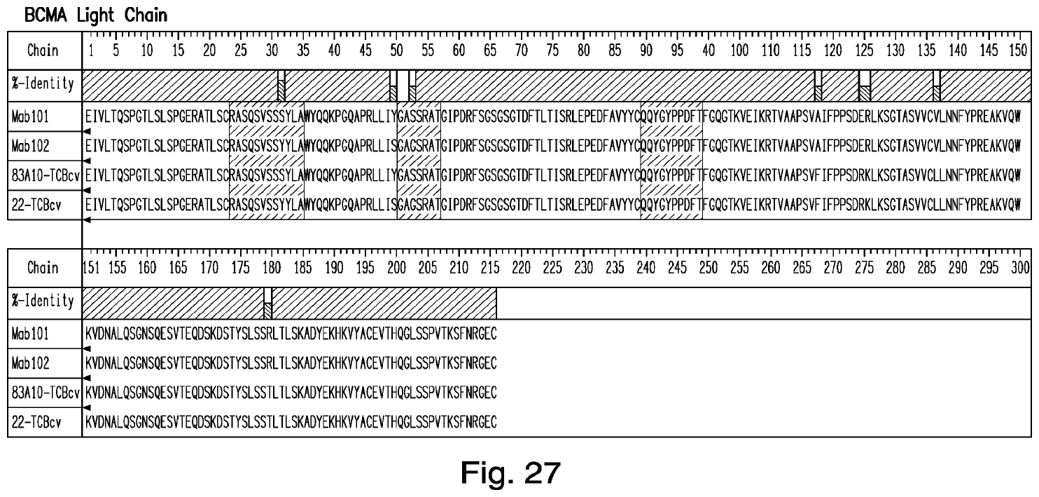

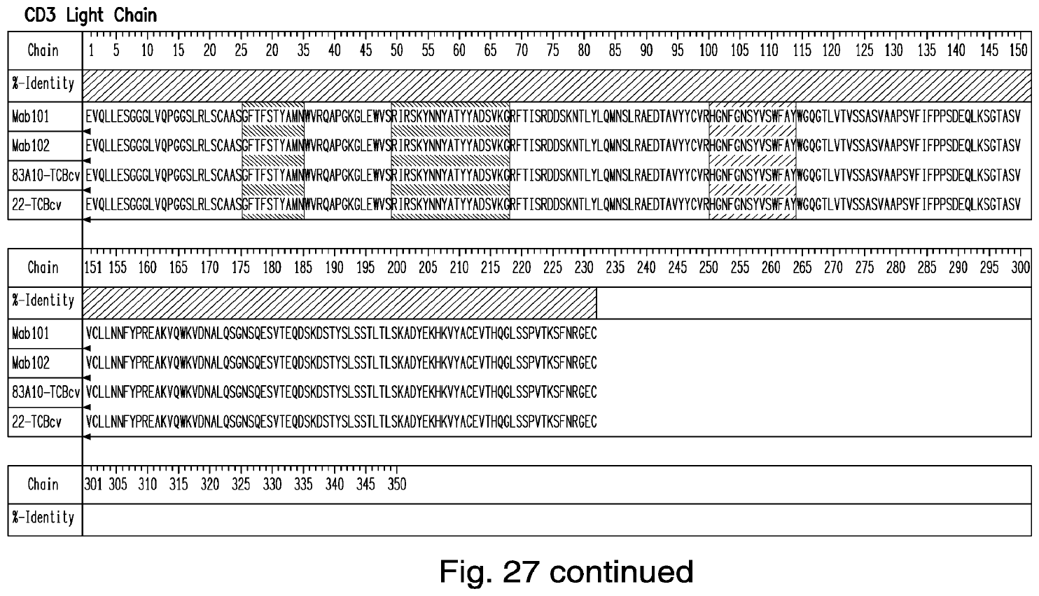

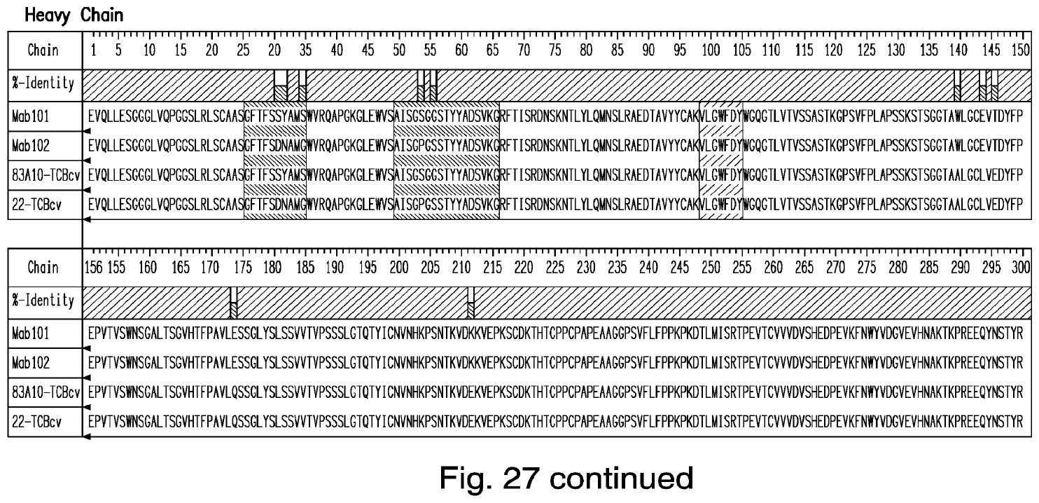

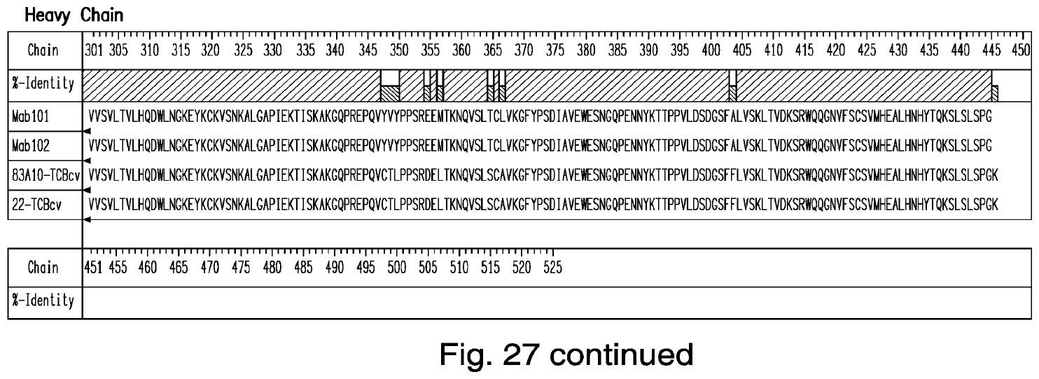

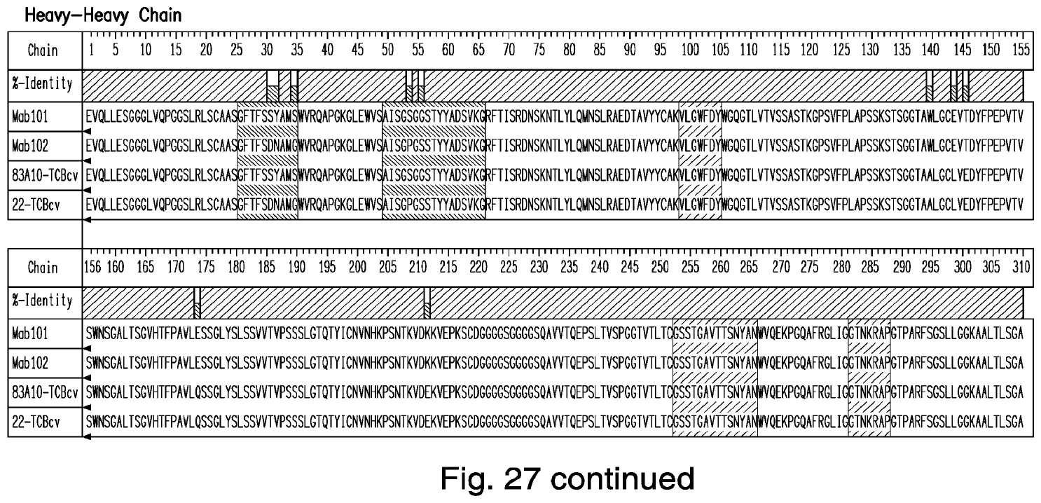

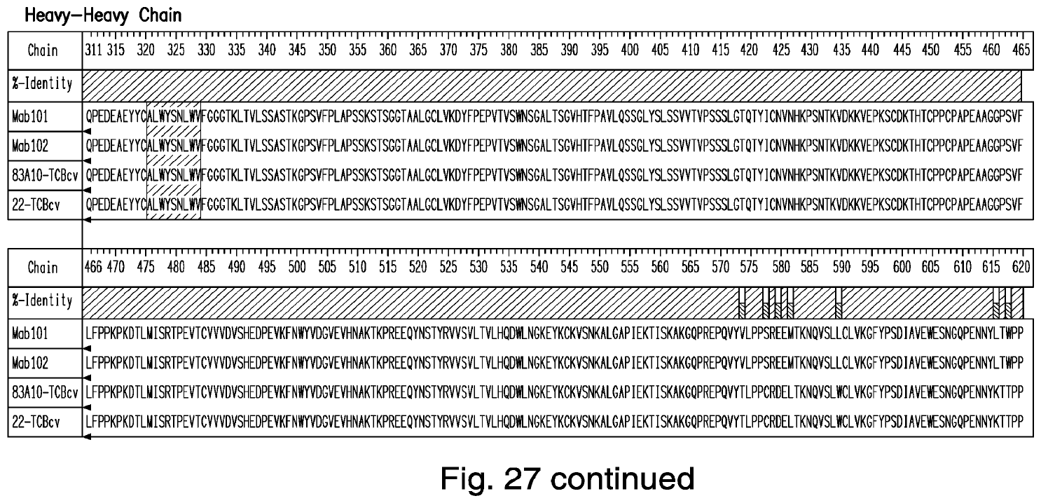

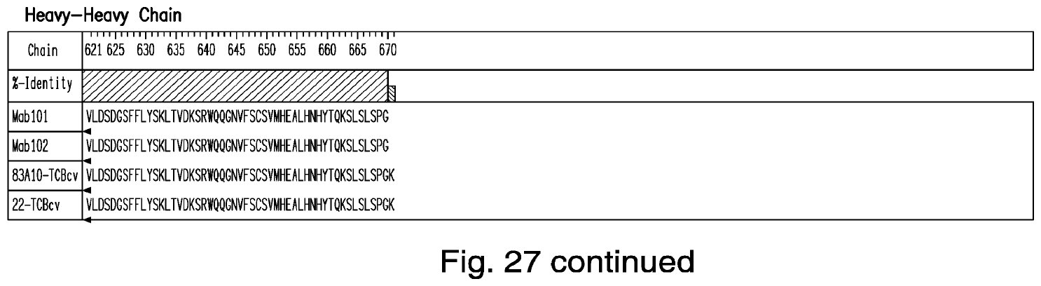

The physicochemical properties of four BCMA×CD3 molecules: Mab101, Mab102, 83A10-TCBcv and 22-TCBcv were evaluated. Mab101 and 83A10-TCBcv comprise a BCMA binding domain comprising the CDRs of antibody 83A10 and Mab102 and 22-TCBcv comprise a BCMA binding domain comprising the CDRs of antibody Mab22. All four variants share the same CD3 binding domain. Sequence alignments of the four BCMA×CD3 molecules are shown in FIG. 27. Each molecule is in a 2+1 bispecific format as shown in FIG. 2A but with alternative amino acid substitutions in CL-CH1 to reduce light chain mispairing/side products: A141W, L145E, K147T, Q175E (“WETE”) and F, 116A, Q124R, L135V, T178R (“ARVR”) rather than the “RK/EE” substitutions illustrated.

The terms “HD1” and “HD1 platform” are used in these Examples to refer to a bispecific antibody comprising “knob-into-hole” mutations. Specifically, the terms “HD1 format” and “HD1 platform” as used herein refer to a bispecific antibody in the format BCMA Fab-Fc-CD3 Fab-BCMA Fab, wherein:
(i) the anti-CD3 Fab fragment comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1;
(ii) the anti-BCMA Fab comprises amino acid substitutions 123R and 124K in the CL domain and amino acid substitutions 147E and 213E in the corresponding CH1 domain;
(iii) the first CH3 domain of the Fc comprises the modification T366W, and the second CH3 domain of the Fc comprises the modifications T366S, L368A, and Y407V.

The terms “HD2 format” and “HD2 platform” are used in these Examples to refer to a bispecific antibody comprising the heterodimerization mutations of the present invention. Specifically, the terms “HD2 format” and “HD2 platform” as used herein refer to a bispecific antibody in the format BCMA Fab-Fc-CD3 Fab-BCMA Fab, wherein:
(i) the anti-CD3 Fab fragment comprises a light chain and a heavy chain, wherein the light chain is a crossover light chain that comprises a variable domain VH and a constant domain CL, and wherein the heavy chain is a crossover heavy chain that comprises a variable domain VL and a constant domain CH1;
(ii) the anti-BCMA Fab comprises amino acid substitutions A141W, L145E, K147T, Q175E in the CH1 domain and amino acid substitutions F116A, Q124R, L135V, T178R in the corresponding CL domain;
(iii) the first CH3 domain of the Fc comprises the modifications T350V, L351Y, F405A and Y407V and the second CH3 domain of the Fc comprises the modifications T350V, T366L, K392L and T394W (numbered according to EU numbering).

As indicated in Table 14, bispecific antibodies Mab101 and Mab102 contain the HD2 mutations of the present invention, while bispecific antibodies 83A10-TCBcv and 22-TCBcv contain “knob-into-hole” (HD1) mutations. “Knob-into-hole” modifications are described in detail with several examples in e.g. WO 96/027011, Ridgway, J. B., et al., Protein Eng. 9 (1996) 617-621, Merchant, A. M. et al., Nat. Biotechnol. 16 (1998) 677-68, and WO 98/050431. These modifications consist of a first CH3 domain comprising the modification T366W (“knob modification”), and a second CH3 domain comprising the modifications T366S, L368A, and Y407V (“hole modifications”) (numbered according to EU numbering of Kabat).

TABLE 14
Antibodies

| | Heterodimerization | BCMA | CD3 |
|---|---|---|---|
| Antibody | platform | binder | binder |
| 83A10-TCBcv | HD1 | 83A10 | CH2527 |
| 22-TCBcv | HD1 | Mab22 | CH2527 |
| Mab101 | HD2 | 83A10 | CH2527 |
| Mab102 | HD2 | Mab22 | CH2527 |

The inventors have shown that the binding capability of the bispecific antibodies in the format of the present invention (i.e. Mab101 and Mab102) as determined by Surface Plasmon Resonance, is less affected by chemical stress (i.e. low pH exposure, high pH exposure and tert-butyl peroxide exposure) than the binding capability of the bispecific antibodies comprising the corresponding BCMA binding domains in the HD1 format (i.e. 83A10-TCBcv and 22-TCBcv), thereby demonstrating that the HD2 format contributes to an overall increase in stability.

The inventors have further shown that the use of the HD2 format compensates for the reduction in physical stability resulting from the CDRs of Mab22. Specifically, measurements of protein concentration by size exclusion chromatography (SEC) show a clear reduction in protein concentration for the HD1 bispecific 22-TCBcv, following both agitation and low pH exposure, that was not observed for the HD2 platform equivalent, Mab102. The use of the HD2 platform in the equivalent Mab102 molecule therefore reduces the negative impact of the Mab22 CDRs on antibody stability.

Furthermore, an overall stability score for each molecule was calculated based on a combined analysis of the data generated by multiple physical and chemical stability assays (see Table 18). In this analysis, both HD2 platform molecules, Mab101 and Mab102, scored higher than the respective HD1 platform equivalents, 83A10-TCBcv and 22-TCBcv suggesting both bispecific molecules to be more stable in the HD2 format.

### Example 18.1: Chemical Stability

The chemical stability assessment consisted of a low pH hold (at pH 4) to accelerate aspartic acid isomerization and fragmentation reactions and a high pH hold (at pH 8) to accelerate asparagine deamidation, oxidation reactions and thioether formation. Tert-butyl peroxide (TBP) was also added to a pH 6 platform buffer to promote oxidation of solvent exposed methionine residues.

The primary methods used to assess chemical stability were surface plasmon resonance (SPR), using the sensorgram comparison method described herein, and size exclusion chromatography (SEC) for instances when chemical modifications may impact physical stability or lead to low molecular weight (LMW) clipping. Peptide mapping was also employed for selected samples depending on the SPR binding results.

### Example 18.1.1 Analysis of Changes in BCMA and CD3 Binding by Surface Plasmon Resonance

SPR experiments were performed using a Biacore T200 system (GE Healthcare, Uppsala, Sweden) with analysis and sample compartment temperatures set at 25° C. and 7° C. respectively.

Anti-Human IgG (from anti-human IgG Capture Kit) was amine coupled to the surface of a CM5 chip according to the manufacturer's instructions. Molecule bispecific antibodies diluted to 1 μg/mL in running buffer were captured on the chip surface in a 60 s injection. Following the single-cycle kinetics procedure, antigen was next injected five times (30 s per injection) at incrementally increasing concentrations at a flow rate of 30 μL/min. Antigen concentrations ranged from 0.08 to 50 nM for BCMA and between 12.7 to 1000 nM for CD3. A 300 s dissociation time was added after the last antigen injection. Following each experiment, all flow cells were regenerated using a 30 s injection of 3M MgCl₂.

Prior to analysis, data were double referenced by first subtracting data from a reference flow cell and then subtracting a blank cycle where buffer was injected instead of antigen. The sensorgram comparison analysis was implemented using a Biacore T200 (software version 3.0). All data were normalized with respect to the response where 100% reflects the maximum binding obtained during injection and 0% reflects the baseline. Average and ±3 standard deviation (SD) sensorgrams were calculated from ten or more independent sensorgrams of each molecule's non-stressed material (standard). For quantitation of binding similarities of the stressed samples against the corresponding non-stressed standard, sample sensorgrams were co-evaluated with the sensorgrams of the standard. The degree of similarity was calculated based on how many sample data points fell inside or outside the ±3 standard deviation limits according to Equation 2, where SSQ is the sum of squares (Karlsson, R, Pol, E., and Frostell, A. (2016); Analytical Biochemistry 502, 53-63). All materials for the SPR analysis are given in Table 15.

TABLE 15
SPR materials

| Material | Detail | Source |
|---|---|---|
| Sensor Chip | Series S CM5 | GE |
| | | Healthcare |
| Running | HBS-EP + buffer (10 mM Hepes, 0.15 | GE |
| Buffer | M NaCl, 3 mM | Healthcare |
| | ethylenediaminetetraacetic acid [EDTA], | |
| | and 0.05% [v/v] surfactant P20, pH 7.4) | |
| Capture | Amine Coupling | GE |
| Method | | Healthcare |
| Capture | Anti-human lgG (Fc) | GE |
| solution | | Healthcare |
| Antibody | TPP-2204, 2205, 2261, and 2253 at | Celgene |
| sample | 1 ug/mL | |
| Antigen 1 | Human BCMA, TPP208 | Celgene |
| | (0.08, 0.4, 2, 10, 50 nM) | |
| Antigen 2 | Human CD3ED, TPP401 | Celgene |
| | (12.3, 37, 111, 333, 1000 nM) | |

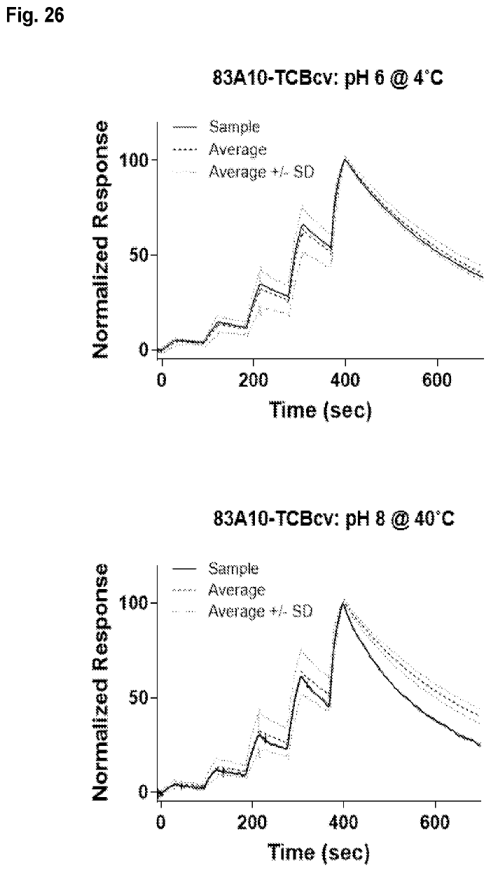

Representative SPR sensorgrams comparing variant 83A10-TCBcv stored for 2 weeks at 2-8° C. (pH 6) and at 40° C. (pH 8) are shown in FIG. 26A and FIG. 26B respectively.

$$Similarity score = \% points inside limits + \% points outside limits ⨯ \frac{SSQ limit distance to average}{SSQ limit distance to average} \qquad Equation 2$$

As shown in Table 18, 22-TCBcv (comprising the HD1 platform mutations) showed the greatest overall reduction CD3 and BCMA binding across all stress conditions except tert-butyl peroxide exposure, and had the lowest overall chemical stability score. Conversely, Mab101 (comprising the HD2 platform mutations of the present invention) showed the least change in binding across all stress conditions.

Both bispecific antibodies comprising the HD2 mutations, (i.e., Mab101 and Mab102), had a greater overall chemically stability score than the corresponding HD1 molecules comprising the same CDR regions, suggesting the HD2 platform contributes to an increase in the chemical stability of the antibody.

The SPR binding data thus suggests that the binding capability of the bispecific antibodies comprising the HD2 mutations (i.e. Mab101 and Mab102) is less affected by chemical stress (i.e. low pH hold, high pH hold and tert-butyl peroxide exposure) than the binding capability of the bispecific antibodies comprising the corresponding BCMA binding domains with the HD1 mutations, (i.e. 83A10-TCBcv and 22-TCBcv). Accordingly, these data indicate that the HD2 mutations of the present invention improve the stability of bispecific antibodies over the HD1 mutations when used in connection with CD3×BCMA bispecific antibodies comprising the CDRs of 83A10 or Mab22.

### Example 18.1.2 Reduced Peptide Mapping

To further distinguish the chemical stabilities of the bispecific antibodies, reduced peptide mapping was performed on all four bispecific antibodies. Molecules were stored for two weeks at 40° C. buffered at pH 8 and at pH 6 in the presence of TBP. As a control, pH 6 samples stored for two weeks at 2-8° C. were also analysed.

All samples were buffer exchanged into 50 mM Acetate, pH 5.0 buffer using a 10-kDa MWCO filter. Next, samples were digested according to the manufacturer suggested AccuMAP protocol (Promego, Madison, WI). Briefly, the samples were denatured by GdHCl, reduced by TCEP, and alkylated by iodoacetamide. A one-hour pre-digest was performed by LysC, followed by dilution of the GdHCl to less than 1 M and addition of methionine to a concentration of 15 mM. A Trypsin/LysC mixture was then added for the final 3-hour digest at 37° C. All steps were performed at pH 5. Digestion was quenched by adding TFA to a final composition of 2%.

Approximately 30 pg of digested sample was injected onto a Waters CSH C18 column (2.1×150 mm, 130 Å pore radius, 1.7 μm bead diameter) in-line with a Orbitrap Velos Pro mass spectrometer (Thermo Fisher Scientific, Waltham, MA). LC Solvent A was 0.1% formic acid (FA) in water, and Solvent B was 0.1% FA in ACN. The separation gradient began at 1% B, increased linearly to 27% B over 110 minutes, and then followed with a five minute linear gradient from 30% to 40% B. The column temperature was set to 70° C. Mass spectra were acquired in a Top-10 data-dependent acquisition. MS1 was performed in the orbitrap with resolving power set to 60,000 at 400 m/z. CID MS/MS was analyzed in the ion trap under rapid scan settings. Dynamic exclusion was set to 15 seconds and a 10 ppm mass window. Raw data was analyzed by Protein Metrics Byonic and Byologic software packages. Modification ratios were calculated as follows using XIC intensity: modified pep intensity/(modified pep intensity+unmodified pep intensity)×100%.

Tables 10 and 11 show the modifications on the residues within or nearby the BCMA and CD3 CDRs, respectively. Consistent with the SPR data, 22-TCBcv showed the largest propensity toward chemical modification in the BCMA and CD3 CDR regions, particularly for methionine and tryptophan oxidation. In contrast, the only modification which was observed at a greater level in the Mab101 molecule was M34 in the BCMA HC and HHC following TBP treatment (Table 16).

TABLE 16
Modifications near BCMA CDRs

| | | | | Modification Ratio (%) | | | | | | | | | | | |
|---|---|---|---|---|---|---|---|---|---|---|---|---|---|---|---|
| | Modifications near BCMA CDRs | | | Mab101 | | | Mab102 | | | 83A10-TCBcv | | | 22-TCBcv | | |
| Chain | Residue(s) | Mod(s) | Distance | pH6 | pH8 | TBP | pH6 | pH8 | TBP | pH6 | pH8 | TBP | pH6 | pH8 | TBP |
| HC/HHC | M34 | Ox | 0 | 0.9 | 1.0 | 10.4 | 1.1 | 0.9 | 6.0 | 0.9 | 1.1 | 6.5 | 1.0 | 1.0 | 4.7 |
| HC/HHC | W36 | Diox | +1 | 0.2 | 0.3 | 0.3 | 0.2 | 0.1 | 0.2 | 0.4 | 0.3 | 0.3 | 0.1 | 0.2 | 0.2 |
| HC/HHC | M34 + W36 | Ox + Diox | 0, +1 | 0.4 | 0.6 | 1.6 | 0.3 | 0.2 | 0.6 | 0.1 | 0.0 | 0.4 | 0.2 | 0.2 | 0.7 |
| HC/HHC | W47 | Diox | −3 | 0.4 | 0.5 | 0.9 | 1.5 | 1.1 | 3.0 | 0.7 | 0.6 | 1.1 | 1.1 | 1.4 | 2.1 |
| HC/HHC | (W102, W106) | Ox | 0, +1 | 0.3 | 0.3 | 1.0 | 0.4 | 0.2 | 3.5 | 0.2 | 0.3 | 1.8 | 0.2 | 0.3 | 5.3 |
| LC1 | W36 | Ox | +1 | 0.1 | 0.1 | 0.1 | 0.2 | 0.1 | 1.9 | 0.1 | 0.1 | 0.5 | 0.2 | 0.1 | 3.4 |
| LC1 | W36 | Diox | +1 | 0.8 | 0.9 | 1.4 | 1.1 | 0.8 | 1.9 | 1.4 | 1.1 | 1.2 | 1.1 | 1.2 | 1.7 |

0 Only one of the residues is modified
+ Both residues exist in modified form together

TABLE 17
Modifications near CD3 CDRs

| | | | | Modification Ratio (%) | | | | | | | | | | | |
|---|---|---|---|---|---|---|---|---|---|---|---|---|---|---|---|
| | Modifications Near CDR3 CDRs | | | Mab101 | | | Mab102 | | | 83A10-TCBcv | | | 22-TCBcv | | |
| Chain | Residue(s) | Mod(s) | Distance | pH6 | pH8 | TBP | pH6 | pH8 | TBP | pH6 | pH8 | TBP | pH6 | pH8 | TBP |
| HHC | (W323, W327) | Diox | (0, 0) | 2.7 | 2.5 | 10.5 | 6.9 | 6.1 | 25.0 | 3.0 | 2.3 | 24.3 | 2.1 | 2.6 | 26.2 |
| LC2b | M34 | Ox | 0 | 0.5 | 1.2 | 22.8 | 0.9 | 1.0 | 27.5 | 1.0 | 1.9 | 31.3 | 0.9 | 1.1 | 40.7 |
| LC2b | W36 | Diox | +1 | — | — | — | 0.4 | 0.6 | 5.1 | — | — | — | 0.5 | 0.0 | 13.2 |
| LC2b | M34 + W36 | Ox + Diox | 0, +1 | 0.5 | 0.8 | 2.9 | 1.2 | 1.1 | 5.5 | 0.9 | 1.2 | 7.2 | 1.1 | 1.3 | 12.7 |
| LC2b | M34 + W36 | Ox + Triox | 0, +1 | 0.0 | 0.0 | 2.8 | 0.2 | 0.2 | 4.5 | 0.1 | 0.0 | 4.6 | 0.0 | 0.0 | 7.1 |
| LC2b | W47 | Ox | −3 | 0.2 | 0.3 | 10.0 | 0.4 | 0.2 | 18.9 | 0.2 | 0.4 | 19.8 | 0.2 | 0.2 | 23.8 |
| LC2b | W47 | Diox | −3 | 3.2 | 3.5 | 15.6 | 7.3 | 5.5 | 26.8 | 5.3 | 4.5 | 27.4 | 5.6 | 5.3 | 31.9 |
| LC2b | (N56, N57) | Deamid | (0,0) | 0.0 | 1.2 | 0.0 | 0.0 | 1.7 | 0.6 | 0.0 | 1.6 | 0.5 | 0.0 | 1.2 | 0.8 |

0 Only one of the residues is modified
+ Both residues exist in modified form together
− Not identified in this sample

### Example 18.2: Physical Stability

The physical stability assessment consisted of measuring thermal stability by differential scanning calorimetery (DSC) and colloidal stability by polyethylene glycol (PEG) precipitation in the platform pH 6 buffer. Physical stability was also assessed following agitation and freeze thaw (F/T) stresses in the pH 6 platform buffer. Finally, a brief low pH hold at room temperature was used to mimic viral inactivation. This processing step that often results in non-native aggregation for less conformationally stable protein biologics.

### Example 18.2.1 Assessment of the Thermal Stability by Differential Scanning Calorimetry

Differential scanning calorimetry was performed on a TA Instruments NanoDSC (New Castle, DE) using the NanoAnalyze software. The BCMA×CD3 samples were diluted to 1 mg/mL in 100 mM histidine pH 6.0 buffer (Table 19). Prior to analysis the samples and their corresponding buffers without protein were degassed for 30 minutes. Samples and buffers were heated from 10 to 100° C. at a rate of 1° C.·min⁻¹. Following acquisition, the corresponding buffers were subtracted from each of the samples and the data was normalized to convert to kcal·mol⁻¹·° C.⁻¹. Results are reported as the temperature (° C.) at the onset of the first unfolding transition.

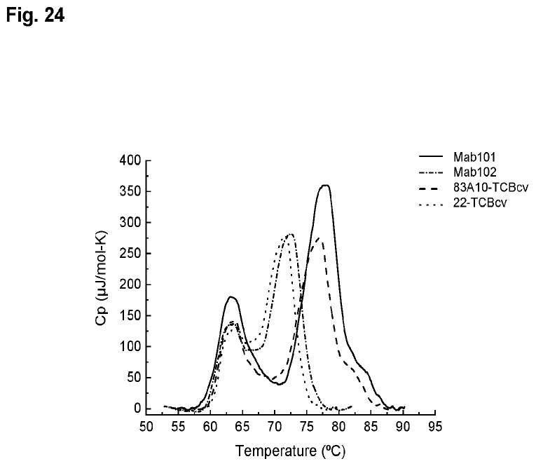

As shown in FIG. 24, all four molecules have similar thermal unfolding onset temperatures (˜60° C.), however the unfolding transition of the most endothermic domains have Tm^{app} values roughly 5° C. greater in the Mab101 and 83A10-TCBcv molecules (comprising the BCMA CDRs of 83A10).

### Example 18.2.2 Colloidal Stability Assessment by PEG Precipitation

A colloidal stability assessment of the four BCMA×CD3 molecules was carried out by PEG 6000 precipitation. PEG precipitation experiments were performed by preparing 160 μL solutions consisting of 1.0 g/mL BCMA×CD3 molecule buffered at pH 6.0 by 100 mM histidine in incrementally increasing concentrations of PEG-6000 from a 40% (w/v) stock solution (Table 19). The solutions were placed at 4° C. overnight and then centrifuged for 60 minutes at 25,000 RCF. The amount of protein remaining in the supernatant was then measured by absorbance at 280 nm using an Agilent Cary UV-8454 (Agilent, Palo Alto, CA). Data were fit to the empirical four parameter sigmoidal equation (Equation 1) to determine and report the percentage (w/v) of PEG-6000 needed to precipitate half of the starting amount of protein (Cm). The parameters b, m and r represent the curves base, maximum value and rate, respectively.

$$y = b + [ \frac{m}{( 1 + exp ( \frac{C m - x}{r} ) )} ] \qquad Equation 1$$

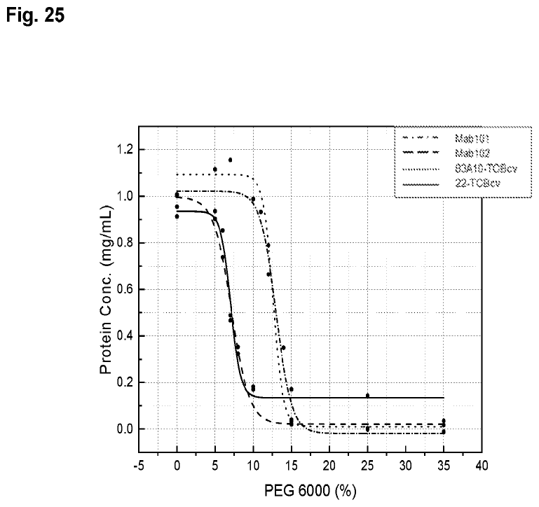

As shown in FIG. 25, Mab102 and 22-TCBcv (comprising the BCMA CDRs of Mab22) had the lowest colloidal stability in the platform pH 6 histidine buffers as evaluated by PEG precipitation. Mab101 and 83A10-TCBcv (comprising the BCMA CDRs of 83A10) required nearly twice as much PEG to induce native state precipitation by excluded volume effects.

### Example 18.2.3 Size Exclusion Chromatography Assessment of Physical Stability Following Agitation and Freeze-Thaw

Physical stability was also assessed by Size Exclusion Chromatography following agitation and freeze thaw (F/T) stresses in the pH 6 platform buffer.

Freeze-Thaw

400 μL of each of the 1 mg/mL BCMA×CD3 molecules (Table 19) were dispensed into 0.5 mL free standing Fisherbrand screw cap cryo tube (part #02-707-357) and stored in a single tier sample box with tube dividers and frozen at −80° C. A total of five freeze-thaw (F/T) cycles were performed where each cycle consisted of freezing samples for at least one hour prior to thaw at room temperature and gently mixing prior to the next F/T cycle. Samples were analysed by SEC following the fifth F/T cycle.

Agitation

400 μL of each of the 1 mg/mL BCMA×CD3 molecules (Table 19) were transferred to 1.5 mL standard Eppendorf tubes (part #022364111) and placed on a VWR microplate shaker with temp control. The shaker was set to 1500 rpm at 25° C. for 24 hours. Post agitation the samples were analyzed by SEC.

Size Exclusion Chromatography

Size Exclusion Chromatography (SEC) was carried out on an Agilent 1260 HPLC system (Agilent, Palo Alto, California) equipped with a single TSKgel G3000SWxl 7.8×300 mm, 5 μm column. The mobile phase consisted of 100 mM KH2PO4, 250 mM NaCl pH 6.8. Twenty microliters of each sample was injected onto the column at 25° C. and eluted isocratically at a flow rate of 0.5 mL/min for 30 minutes. Elution was monitored by UV detection at 280 nm and protein concentration was calculated using the total area of the integrated curve, flow rate, injection volume, flow cell path length and the extinction coefficients of each the BCMA×CD3 molecules.

Two values are reported for SEC analysis; the decrease in % monomer and the concentration relative to the pH 6 2 wk sample held at 2-8° C. This methodology decreases the time and material requirements needed by eliminating “time zero” samples as well as minimizing potential chromatography variabilities (such as column performance) by analyzing all samples within the same chromatography sequence.

To account for potential dilution variability (as well as integration and injection variability) ten independent 1:10 dilutions from the same 10 mg/mL standard mAb solution were also prepared and analyzed them in the same SEC sequence as the BCMA×CD3 stability samples. The decrease in the percentage of monomer and concentration is reported in relation to two times the standard deviation (2σ) of the average percent monomer (2σM) and concentration (2σC) of these dilution standards, respectively.

The results from the SEC assessment are provided in Table 15 and the differences in the percent main peak monomer and concentration between the pH 6 2 k 2-8° C. (control) sample and each of the stressed samples are shown in Table 18.

Consistent with the SPR data, the SEC results indicate that there was a clear reduction in protein concentration for HD1 platform molecule 22-TCBcv following agitation after the pH 3 hold that was not observed for the other molecules. Thus, the use of the HD2 platform in the equivalent Mab101 molecule minimizes the negative impact of the Mab22 CDRs on stability. A representative chromatogram of the 22-TCBcv variant is shown in FIG. 26.

Scoring

Molecules were scored according to acceptance criteria for each of the physical and chemical stability indicating method responses (light grey shaded region of Table 18) and assigned a score of 0, 1 or 2 based on how the experimental results (dark grey shaded regions) fit within these criteria. The decrease in percent monomer and concentrations measured by SEC were scored relative to two times the standard deviations of the average percent monomer (2σM) and concentration (2σC) determined from ten independent 1:10 dilutions of a standard 10 mg/mL mAb solution; for the current study 2σM and 2σC were 0.33 and 0.14, respectively. The sums of the physical and chemical stability scores were divided by their respective number of responses (8 for physical and 16 for the chemical assessment) so that both physical and chemical stability scores range from 0-2. The total score for each variant is then the sum of the physical and chemical stability scores and therefore ranges from 0-4 (Table 18). All responses in Table 18 are weighted equally.

Table 18 shows that in the physical stability assessment, there was a clear reduction in protein concentration for 22-TCBcv molecule following agitation and after a pH 3 hold, that was not observed for the other molecules.

22-TCBcv also underperformed the other molecules in the chemical stability assessment, displaying greater losses in percent monomer following oxidation by TBP, and greater losses in CD3 and BCMA binding after low and high pH holds. As in the physical stability portion of the assessment, Mab101 showed the least change in concentration, percent monomer and binding following all chemical stresses. Both HD2-platform molecules, i.e. Mab101 and Mab102 scored higher than the respective HD1 platform equivalents (Table 18).

Therefore, these data clearly demonstrate that the HD2-platform format resulted in a more stable molecule than the HD1 platform format for both the 83A10 and Mab22 bispecific antibodies.

TABLE 18

| Physical and | | | | | | | | | | | |
|---|---|---|---|---|---|---|---|---|---|---|---|
| Chemical | | | | | | | | | | | |
| Stability Scoring | | | | | | | | | score | | score |
| of | scoring | | | | score | | score | 83A10- | 83A10- | 22- | 22- |
| BCMAxCD3 | 0 | 1 | 2 | Mab101 | Mab101 | Mab102 | Mab102 | TCBcv | TCBcv | TCBcv | TCBcv |
| Physical Stability Method | | | | | | | | | | | |
| pH 6 DSC onset Tm1 (° C.) | <50 | 50-55 | >55 | 59.30 | 2 | 59.20 | 2 | 60.00 | 2 | 60.10 | 2 |
| pH 6 PEG-6000 Cm (% w/v) | <10 | 10-20 | >20 | 12.20 | 1 | 8.60 | 0 | 14.40 | 1 | 4.10 | 0 |
| pH 6-80° C. F/T decrease in | >5 | >2σM − | ≤2σM | 0.37 | 1 | −2.40 | 2 | 1.42 | 1 | −1.33 | 2 |
| % main¹ | | 5 | | | | | | | | | |
| pH 6-80° C. F/T decrease in | >2σC | — | ≤2σC | −0.07 | 2 | 0.02 | 2 | −0.01 | 2 | 0.01 | 2 |
| mg/ml | | | | | | | | | | | |
| pH 6 RT agitation decrease | >10 | 5-10 | <5 | 0.02 | 2 | −1.53 | 2 | 1.20 | 2 | −1.98 | 2 |
| in | | | | | | | | | | | |
| % main | | | | | | | | | | | |
| pH 6 RT agitation decrease | >2σC | — | ≤2σC | 0.03 | 2 | -0.09 | 2 | 0.00 | 2 | 0.29 | 0 |
| in mg/mL | | | | | | | | | | | |
| pH 3, 3 hr. decrease in % | >10 | 5-10 | <5 | 3.40 | 2 | 5.32 | 1 | 3.09 | 2 | 1.47 | 2 |
| main | | | | | | | | | | | |
| pH 3, 3 hr. decrease in | >2σC | — | ≤2σC | 0.04 | 2 | 0.07 | 2 | −0.01 | 2 | 0.23 | 0 |
| mg/mL | | | | | | | | | | | |
| Physical Stability Scores | | | | | 1.75 | | 1.63 | | 1.75 | | 1.25 |
| Chemical Stability Method² | | | | | | | | | | | |
| pH 6 decrease in % main | >10 | 5-10 | <5 | −0.22 | 2 | −1.57 | 2 | 0.11 | 2 | 0.96 | 2 |
| pH 6 decrease in mg/ml | >2σC | — | ≤2σC | 0.02 | 2 | 0.05 | 2 | 0.03 | 2 | 0.04 | 2 |
| pH 6 SPR % similarity- | <90 | 90-95 | >95 | 99.99 | 2 | 99.44 | 2 | 99.96 | 2 | 99.85 | 2 |
| BCMA | | | | | | | | | | | |
| pH 6 SPR % similarity-CD3 | <90 | 90-95 | >95 | 99.96 | 2 | 99.95 | 2 | 99.90 | 2 | 99.50 | 2 |
| pH 6-TBP decrease in % | >10 | 5-10 | <5 | 9.12 | 1 | 11.62 | 0 | 12.40 | 0 | 21.03 | 0 |
| main | | | | | | | | | | | |
| pH 6-TBP decrease in | >2σC | — | ≤2σC | 0.02 | 2 | 0.08 | 2 | 0.06 | 2 | 0.06 | 2 |
| mg/ml | | | | | | | | | | | |
| pH 6-TBP SPR % similarity- | <80 | 80-90 | >90 | 82.50 | 1 | 54.00 | 0 | 72.00 | 0 | 71.93 | 0 |
| BCMA | | | | | | | | | | | |
| pH 6-TBP SPR % similarity- | <80 | 80-90 | >90 | 75.30 | 0 | 72.30 | 0 | 59.70 | 0 | 58.50 | 0 |
| CD3 | | | | | | | | | | | |
| pH 4 decrease in % main | >10 | 5-10 | <5 | 9.12 | 1 | 8.73 | 1 | 7.97 | 1 | 9.38 | 1 |
| pH 4 decrease in mg/mL | >2σC | — | ≤2σC | −0.06 | 2 | 0.07 | 2 | 0.04 | 2 | −0.07 | 2 |
| pH 4 SPR % similarity- | <80 | 80-90 | >90 | 99.99 | 2 | 99.98 | 2 | 99.42 | 2 | 84.76 | 1 |
| BCMA | | | | | | | | | | | |
| pH 4 SPR % similarity-CD3 | <80 | 80-90 | >90 | 99.93 | 2 | 99.98 | 2 | 100 | 2 | 99.60 | 2 |
| pH 8 decrease in % main | >10 | 5-10 | <5 | 7.83 | 1 | 9.08 | 1 | 8.88 | 1 | 9.00 | 1 |
| pH 8 decrease in mg/ml | >2σC | — | ≤2σC | −0.04 | 2 | 0.13 | 2 | 0.12 | 2 | 0.04 | 2 |
| pH 8 SPR % similarity- | <80 | 80-90 | >90 | 99.92 | 2 | 95.70 | 2 | 90.80 | 2 | 95.81 | 2 |
| BCMA | | | | | | | | | | | |
| pH 8 SPR % similarity-CD3 | <80 | 80-90 | >90 | 71.50 | 0 | 70.11 | 0 | 57.48 | 0 | 59.50 | 0 |
| Chemical Stability Scores | | | | | 1.50 | | 1.38 | | 1.38 | | 1.31 |
| Total Developability | | | | | | | | | | | |
| Scores | | | | | 3.25 | | 3.00 | | 3.13 | | 2.56 |

¹Note that the acceptance criteria for the loss in SEC % main peak monomer for F/T is more stringent than for all other stress conditions.
²The storage temperature for all chemical stability methods was 40° C.

TABLE 19
Formulation spreadsheet describing how each candidate was formulated.

| Variant #1 | Sample ID: | Mab101 | | | concentration after buffer exchange | | | | 13 |
|---|---|---|---|---|---|---|---|---|---|
| Formulations for chemical stability, F/T, DSC and agitation | | | | | (mg/mL) | | | | |
| studies | | | | | PEG precipitation screen | | | | |
| | | | volume | | | | volume | | |
| | | | of | | | volume | 40 % | | |
| | | | 100 | | % | of | (w/v) | volume | |
| | volume of | TBP | mM | final | PEG- | variant | PEG | 100 mM | final |
| | variant #1 | stock | buffers | volume | 6000 | #1 | stock | His pH 6 | volume |
| Formulation | (mL) | (μL) | (mL) | (mL) | (w/v) | (mL) | (mL) | (mL) | (mL) |
| pH 6 | 0.177 | — | 2.123 | 2.300 | 0 | 12 | 0 | 148 | 160 |
| pH 6 TBP | — | 3.000 | — | 0.303 | 5 | 12 | 20 | 128 | 160 |
| pH 4 | 0.023 | — | 0.277 | 0.300 | 10 | 12 | 40 | 108 | 160 |
| pH 8 | 0.023 | — | 0.277 | 0.300 | 15 | 12 | 60 | 88 | 160 |
| pH 3 | 0.023 | — | 0.277 | 0.300 | 20 | 12 | 80 | 68 | 160 |
| The 2.3 mL of pH 6.0 formulation should be split into 5 × 0.3 mL | | | | | 25 | 12 | 100 | 48 | 160 |
| aliquots and 2 × 0.4 mL aliquots. 3.0 μL of 0.5M TBP should be | | | | | 30 | 12 | 120 | 28 | 160 |
| spiked into one of the 0.3 mL aliquots. The 0.4 mL aliquots will | | | | | 35 | 12 | 140 | 8 | 160 |
| be used for F/T and agitation studies. | | | | | | | | | |
| Variant #2 | Sample ID: | Mab102 | | | concentration after buffer exchange | | | | 11.9 |
| Formulations for chemical stability, F/T, DSC and agitation | | | | | (mg/mL) | | | | |
| studies | | | | | PEG precipitation screen | | | | |
| | | | volume | | | | volume | | |
| | | | of | | | volume | 40 % | | |
| | | | 100 | | % | of | (w/v) | volume | |
| | volume of | TBP | mM | final | PEG- | variant | PEG | 100 mM | final |
| | variant #2 | stock | buffers | volume | 6000 | #2 | stock | His pH 6 | volume |
| Formulation | (mL) | (μL) | (mL) | (mL) | (w/v) | (mL) | (mL) | (mL) | (mL) |
| pH 6 | 0.193 | — | 2.107 | 2.300 | 0 | 13 | 0 | 147 | 160 |
| pH 6 TBP | — | 3.000 | — | 0.303 | 5 | 13 | 20 | 127 | 160 |
| pH 4 | 0.025 | — | 0.275 | 0.300 | 10 | 13 | 40 | 107 | 160 |
| pH 8 | 0.025 | — | 0.275 | 0.300 | 15 | 13 | 60 | 87 | 160 |
| pH 3 | 0.025 | — | 0.275 | 0.300 | 20 | 13 | 80 | 67 | 160 |
| The 2.3 mL of pH 6.0 formulation should be split into 5 × 0.3 mL | | | | | 25 | 13 | 100 | 47 | 160 |
| aliquots and 2 × 0.4 mL aliquots. 3.0 μL of 0.5M TBP should be | | | | | 30 | 13 | 120 | 27 | 160 |
| spiked into one of the 0.3 mL aliquots. The 0.4 mL aliquots will | | | | | 35 | 13 | 140 | 7 | 160 |
| be used for F/T and agitation studies. | | | | | | | | | |
| Variant #3 | Sample ID: | 83A10-TCBCV | | | concentration after buffer exchange | | | | 12.8 |
| Formulations for chemical stability, F/T, DSC and agitation | | | | | (mg/mL) | | | | |
| studies | | | | | PEG precipitation screen | | | | |
| | | | volume | | | | volume | | |
| | | | of | | | volume | 40 % | | |
| | | | 100 | | % | of | (w/v) | volume | |
| | volume of | TBP | mM | final | PEG- | variant | PEG | 100 mM | final |
| | variant #3 | stock | buffers | volume | 6000 | #3 | stock | His pH 6 | volume |
| Formulation | (mL) | (μL) | (mL) | (mL) | (w/v) | (mL) | (mL) | (mL) | (mL) |
| pH 6 | 0.180 | — | 2.120 | 2.300 | 0 | 13 | 0 | 148 | 160 |
| pH 6 TBP | — | 3.000 | — | 0.303 | 5 | 13 | 20 | 128 | 160 |
| pH 4 | 0.023 | — | 0.277 | 0.300 | 10 | 13 | 40 | 108 | 160 |
| pH 8 | 0.023 | — | 0.277 | 0.300 | 15 | 13 | 60 | 88 | 160 |
| pH 3 | 0.023 | — | 0.277 | 0.300 | 20 | 13 | 80 | 68 | 160 |
| The 2.3 mL of pH 6.0 formulation should be split into 5 × 0.3 mL | | | | | 25 | 13 | 100 | 48 | 160 |
| aliquots and 2 × 0.4 mL aliquots. 3.0 μL of 0.5M TBP should be | | | | | 30 | 13 | 120 | 28 | 160 |
| spiked into one of the 0.3 mL aliquots. The 0.4 mL aliquots will | | | | | 35 | 13 | 140 | 8 | 160 |
| be used for F/T and agitation studies. | | | | | | | | | |
| Variant #4 | Sample ID: | 22-TCBCV | | | concentration after buffer exchange | | | | 5.4 |
| Formulations for chemical stability, F/T, DSC and agitation | | | | | (mg/mL) | | | | |
| studies | | | | | PEG precipitation screen | | | | |
| | | | volume | | | | volume | | |
| | | | of | | | volume | 40 % | | |
| | | | 100 | | % | of | (w/v) | volume | |
| | volume of | TBP | mM | final | PEG- | variant | PEG | 100 mM | final |
| | variant #4 | stock | buffers | volume | 6000 | #4 | stock | His pH 6 | volume |
| Formulation | (mL) | (μL) | (mL) | (mL) | (w/v) | (mL) | (mL) | (mL) | (mL) |
| pH 6 | 0.426 | — | 1.874 | 2.300 | 0 | 30 | 0 | 130 | 160 |
| pH 6 TBP | — | 3.000 | — | 0.303 | 5 | 30 | 20 | 110 | 160 |
| pH 4 | 0.056 | — | 0.244 | 0.300 | 10 | 30 | 40 | 90 | 160 |
| pH 8 | 0.056 | — | 0.244 | 0.300 | 15 | 30 | 60 | 70 | 160 |
| pH 3 | 0.056 | — | 0.244 | 0.300 | 20 | 30 | 80 | 50 | 160 |

| | | | | | |
|---|---|---|---|---|---|
| The 2.3 mL of pH 6.0 formulation should be split into 5 × 0.3 mL | 25 | 30 | 100 | 30 | 160 |
| aliquots and 2 × 0.4 mL aliquots. 3.0 μL of 0.5M TBP should be | 30 | 30 | 120 | 10 | 160 |
| spiked into one of the 0.3 mL aliquots. The 0.4 mL aliquots will | 35 | — | — | — | — |
| be used for F/T and agitation studies. | | | | | |

TABLE 20
SEC results for all stress conditions of the developability assessment

| | | | | | | | | | | | |
|---|---|---|---|---|---|---|---|---|---|---|---|
| | SEC | SEC | SEC | SEC | | | | | | | |
| | HMW | main | LMW | total | | | | | | | |
| | Area | Area | Area | Area | SEC | SEC | SEC | SEC | | | |
| Stress | (mAu* | (mAu* | (mAu* | (mAu* | % | % | % | (mg/ | | | |
| Condition | min) | min) | min) | min) | HMW | main | LMW | mL) | parameter | value | units |

Sample ID: Mab101

| | | | | | | | | | | | |
|---|---|---|---|---|---|---|---|---|---|---|---|
| pH 6 2-8° C. | 16.5 | 2056.8 | 95.7 | 2169 | 0.761 | 94.827 | 4.412 | 0.984 | extinc. | 1.530 | ml*mg⁻¹ |
| | | | | | | | | | coeff. | | *cm⁻¹ |
| pH 6 40° C. | 12.9 | 2010.8 | 91.8 | 2115.5 | 0.610 | 95.051 | 4.339 | 0.960 | pathlength | 0.6 | cm |
| | | | | | | | | | flow rate | 0.5 | mL/min |
| pH 6-TBP 40° C. | 149 | 1826.6 | 155.7 | 2131.3 | 6.991 | 85.704 | 7.305 | 0.967 | inject. Vol. | 0.020 | mL |
| pH 4 40° C. | 61.2 | 1967.4 | 266.8 | 2295.4 | 2.666 | 85.711 | 11.623 | 1.042 | | | |
| pH 8 40° C. | 107.3 | 1972.2 | 187.5 | 2267 | 4.733 | 86.996 | 8.271 | 1.029 | | | |
| pH 3 3 hr. 25° C. | 74.5 | 1910.6 | 104.6 | 2089.7 | 3.565 | 91.429 | 5.006 | 0.948 | | | |
| pH 6.0-80 | 25 | 2197.7 | 103.9 | 2326.6 | 1.075 | 94.460 | 4.466 | 1.056 | | | |
| F/T 5x | | | | | | | | | | | |
| pH 6.0 RT | 14.4 | 1986.3 | 94.4 | 2095.1 | 0.687 | 94.807 | 4.506 | 0.951 | | | |
| agitation | | | | | | | | | | | |

Sample ID: Mab102

| | | | | | | | | | | | |
|---|---|---|---|---|---|---|---|---|---|---|---|
| pH 6 2-8° C. | 133.8 | 1995.3 | 101.8 | 2230.9 | 5.998 | 89.439 | 4.563 | 1.000 | extinc. | 1.550 | ml*mg⁻¹ |
| | | | | | | | | | coeff. | | *cm⁻¹ |
| pH 6 40° C. | 123 | 1923.2 | 67 | 2113.2 | 5.821 | 91.009 | 3.171 | 0.947 | pathlength | 0.6 | cm |
| | | | | | | | | | flow rate | 0.5 | mL/min |
| pH 6-TBP 40° C. | 289.5 | 1592.1 | 164.3 | 2045.9 | 14.150 | 77.819 | 8.031 | 0.917 | inject. Vol. | 0.020 | mL |
| pH 4 40° C. | 142.2 | 1677.8 | 258.8 | 2078.8 | 6.840 | 80.710 | 12.449 | 0.931 | | | |
| pH 8 40° C. | 139.8 | 1555.6 | 240.3 | 1935.7 | 7.222 | 80.364 | 12.414 | 0.867 | | | |
| pH 3 3 hr. 25° C. | 216.7 | 1739.6 | 111.8 | 2068.1 | 10.478 | 84.116 | 5.406 | 0.927 | | | |
| pH 6.0-80 | 130.7 | 2010.3 | 48 | 2189 | 5.971 | 91.836 | 2.193 | 0.981 | | | |
| F/T 5x | | | | | | | | | | | |
| pH 6.0 RT | 148.6 | 2204 | 70.2 | 2422.8 | 6.133 | 90.969 | 2.897 | 1.085 | | | |
| agitation | | | | | | | | | | | |

Sample ID: 83A10-TCBcv

| | | | | | | | | | | | |
|---|---|---|---|---|---|---|---|---|---|---|---|
| pH 6 2-8° C. | 0 | 2126.5 | 52.7 | 2179.2 | 0.000 | 97.582 | 2.418 | 1.029 | extinc. | 1.470 | ml*mg⁻¹ |
| | | | | | | | | | coeff. | | *cm⁻¹ |
| pH 6 40° C. | 0 | 2054.6 | 53.4 | 2108 | 0.000 | 97.467 | 2.533 | 0.996 | pathlength | 0.6 | cm |
| | | | | | | | | | flow rate | 0.5 | mL/min |
| pH 6-TBP 40° C. | 177 | 1756.9 | 128.6 | 2062.5 | 8.582 | 85.183 | 6.235 | 0.974 | inject. Vol. | 0.020 | mL |
| pH 4 40° C. | 0 | 1875.2 | 217.3 | 2092.5 | 0.000 | 89.615 | 10.385 | 0.989 | | | |
| pH 8 40° C. | 54.8 | 1715.4 | 163.6 | 1933.8 | 2.834 | 88.706 | 8.460 | 0.914 | | | |
| pH 3 3 hr. 25° C. | 47.35 | 2069.45 | 73.3 | 2190.1 | 2.162 | 94.491 | 3.347 | 1.035 | | | |
| pH 6.0-80 | 31.3 | 2118.9 | 53.3 | 2203.5 | 1.420 | 96.161 | 2.419 | 1.041 | | | |
| F/T 5x | | | | | | | | | | | |
| pH 6.0 RT | 21.3 | 2103.3 | 57.6 | 2182.2 | 0.976 | 96.384 | 2.640 | 1.031 | | | |
| agitation | | | | | | | | | | | |

Sample ID: 22-TCBcv

| | | | | | | | | | | | |
|---|---|---|---|---|---|---|---|---|---|---|---|
| pH 6 2-8° C. | 18.1 | 1907.6 | 47.2 | 1972.9 | 0.917 | 96.690 | 2.392 | 0.938 | extinc. | 1.460 | ml*mg⁻¹ |
| | | | | | | | | | coeff. | | *cm⁻¹ |
| pH 6 40° C. | 14.6 | 1811 | 66.2 | 1891.8 | 0.772 | 95.729 | 3.499 | 0.900 | pathlength | 0.6 | cm |
| | | | | | | | | | flow rate | 0.5 | mL/min |
| pH 6-TBP 40° C. | 297.1 | 1393 | 151 | 1841.1 | 16.137 | 75.661 | 8.202 | 0.876 | inject. Vol. | 0.020 | mL |
| pH 4 40° C. | 22.7 | 1844.5 | 245.3 | 2112.5 | 1.075 | 87.314 | 11.612 | 1.005 | | | |
| pH 8 40° C. | 22.5 | 1656.8 | 210 | 1889.3 | 1.191 | 87.694 | 11.115 | 0.899 | | | |
| pH 3 3 hr. 25° C. | 15.2 | 1425.4 | 56.3 | 1496.9 | 1.015 | 95.223 | 3.761 | 0.712 | | | |
| pH 6.0-80 | 21.8 | 1918.7 | 16.9 | 1957.4 | 1.114 | 98.023 | 0.863 | 0.931 | | | |
| F/T 5x | | | | | | | | | | | |
| pH 6.0 RT | 6.8 | 1346.3 | 11.4 | 1364.5 | 0.498 | 98.666 | 0.835 | 0.649 | | | |
| agitation | | | | | | | | | | | |

1. A method of treating or managing an autoimmune disorder, the method comprising administering to a patient in need thereof a multispecific antibody, wherein the multispecific antibody binds to B-cell maturation antigen (BCMA) and an antigen that promotes the activation of one or more T cells.

2. The method of claim 1, wherein the autoimmune disorder is anti-neutrophil cytoplasmic antibody (ANCA)-associated vasculitis (AAV), rheumatoid arthritis (RA), or systemic lupus erythematosus (SLE).

3. The method of claim 1, wherein the autoimmune disease is AAV.

4. The method of claim 1, wherein the antigen that promotes the activation of one or more T cells is selected from the group consisting of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, and CD226.

5. The method of claim 1, wherein the multispecific antibody is a bispecific antibody that binds to BCMA and CD3.

6. The method of claim 5, wherein the bispecific antibody is a trivalent bispecific antibody comprising two Fab fragments of an anti-BCMA antibody (BCMA Fab), one Fab fragment of an anti-CD3 antibody (CD3 Fab), and one Fc portion, and is in a format pf BCMA Fab-Fc-CD3 Fab-BCMA Fab.

7. The method of claim 1, wherein the multispecific antibody comprises an anti-BCMA antibody or an antigen-binding fragment thereof comprising:
a heavy chain complementarity-determining region 1 (CDR1H) comprising the amino acid sequence of SEQ ID NO:21, a heavy chain complementarity-determining region 2 (CDR2H) comprising the amino acid sequence of SEQ ID NO: 22, a heavy chain complementarity-determining region 3 (CDR3H) comprising the amino acid sequence of SEQ ID NO: 17, a light chain complementarity-determining region 1 (CDR1L) comprising the amino acid sequence of SEQ ID NO:27, a light chain complementarity-determining region 2 (CDR2L) comprising the amino acid sequence of SEQ ID NO:28, and a light chain complementarity-determining region 3 (CDR3L) comprising the amino acid sequence of SEQ ID NO:20.

8. The method of claim 1, wherein the multispecific antibody comprises
an anti-BCMA antibody or antigen-binding fragment thereof comprising a heavy chain variable region (VH) comprising an amino acid sequence that is at least 90% identical to, at least 95% identical to, at least 99% identical to, or identical to the amino acid sequence of SEQ ID NO: 10; and a light chain variable region (VL) comprising an amino acid sequence that is at least 90% identical to, at least 95% identical to, at least 99% identical to, or identical to the amino acid sequence of SEQ ID NO: 14.

9. The method of claim 1, wherein the multispecific antibody comprises an anti-CD3 antibody or an antigen-binding fragment thereof comprising a CDR1H comprising the amino acid sequence of SEQ ID NO: 1, a CDR2H comprising the amino acid sequence of SEQ ID NO:2, a CDR3H comprising the amino acid sequence of SEQ ID NO:3, a CDR1L comprising the amino acid sequence of SEQ ID NO: 4, CDR2L comprising the amino acid sequence of SEQ ID NO: 5, and a CDR3L comprising the amino acid sequence of SEQ ID NO:6.

10. The method according to claim 9, wherein the anti-CD3 antibody an antigen-binding fragment thereof comprises a VH comprising an amino acid sequence that is at least 90% identical to, at least 95% identical to, at least 99% identical to, or identical to the amino acid sequence of SEQ ID NO:7; and a VL comprising an amino acid sequence that is at least 90% identical to, at least 95% identical to, at least 99% identical to, or identical to the amino acid sequence of SEQ ID NO: 8.

11. The method of claim 1, wherein the multispecific antibody comprises five polypeptides comprising the amino acid sequence of
SEQ ID NO:48, SEQ ID NO:55, SEQ ID NO:56, SEQ ID NO:57, and SEQ ID NO:57 respectively.

12. The method of claim 1, wherein the autoimmune disease is selected from the group consisting of systemic lupus erythematosus, IgA nephropathy, membranous nephropathy, myasthenia gravis, neuromyelitis optica, pemphigus vulgaris, anti-PAD4-activating rheumatoid arthritis, sensitized/preformed antibodies in solid organ transplant, Guillain-Barre Syndrome, chronic inflammatory demyelinating polyneuropathy (CIDP), immune thrombocytopenic purpura, rheumatoid arthritis, and AAV.

13. The method of claim 3, wherein the AAV is selected from the group consisting of granulomatosis with poly angiitis (GPA), eosinophilic granulomatosis with polyangiitis (EGPA), microscopic polyangiitis (MPA), and renal-limited AAV.

14. (canceled)

15. (canceled)

16. (canceled)

17. (canceled)

18. The method of claim 1, wherein the autoimmune disorder is refractory or relapsed, and/or is newly diagnosed.

19. (canceled)

20. The method of claim 3, wherein the AAV is affecting one or more body parts of the patient selected from nervous system, eyes, nose, heart, kidneys, stomach, intestine, lungs, joints, muscles, and skin.

21. The method of claim 3, wherein the AAV is generalized with presence of life- or major organ-threatening manifestations, or the AAV is localized without organ-threatening manifestations.

22. The method according to claim 21, wherein the patient has diffuse alveolar hemorrhage (DAH).

23. (canceled)

24. The method of claim 1, wherein the patient is (a) in need of plasmablast reduction, (b) at risk of developing cytokine release syndrome, (c) at risk of developing infection, (d) in need of induction of remission, and/or (e) in need of maintenance of remission.

25. (canceled)

26. (canceled)

27. (canceled)

28. (canceled)

29. The method of claim 1, wherein the method results in
(a) a reduction in the number of plasmablasts in the patient by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to the number of plasmablasts in a patient receiving no treatment or a reference treatment;
(b) a lowered incidence of cytokine release syndrome in the patient by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to the incidence of cytokine release syndrome in a patient receiving a reference treatment;
(c) a lowered incidence of infection in the patient at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to the incidence of infection in a patient receiving a reference treatment;
(d) a reduced time of induction of remission in the patient by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to the time of induction of remission in a patient receiving a reference treatment; and/or
(e) an increased time of maintenance of remission in the patient by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% relative to the time of maintenance of remission in a patient receiving a reference treatment.

30. (canceled)

31. (canceled)

32. (canceled)

33. (canceled)

34. The method of claim 29, wherein the reference treatment is selected from the group consisting of steroids, cyclophosphamide, anti-CD20 monoclonal antibodies, methotrexate, azathioprine, mycophenolate, mycophenolate mofetil, avacopan, anti-TNF agents, anti-IL6R antibodies, costimulatory blockade JAK inhibitors, and/or belimumab.

35. The method of claim 1, wherein the method is used for the induction of remission or the maintenance of remission.

36. (canceled)

37. The method of claim 34, wherein the reference treatment is a steroid, cyclophosphamide, or rituximab.

38. The method of claim 34, wherein (a) the steroid is a glucocorticoid, (b) the anti-CD20 monoclonal antibody is rituximab, (c) the anti-TNF agent is infliximab, adalimumab, golimumab, or etanercept), (d) the anti-IL6R antibody is tocilizumab or sarilumab, (e) the costimulatory blockade is abatacept, or (f) JAK inhibitors is tofacitinib or baricitinib.

39. The method of claim 4, wherein the antigen that promotes the activation of one or more T cells is CD3.

40. The method of claim 1, wherein the multispecific antibody comprises an anti-BCMA antibody or an antigen-binding fragment thereof comprising a CDR1H region comprising the amino acid sequence of SEQ ID NO:21, a CDR2H region comprising the amino acid sequence of SEQ ID NO:22, a CDR3H region comprising the amino acid sequence of SEQ ID NO: 17, a CDR1L region comprising the amino acid sequence of SEQ ID NO:25, a CDR2L region comprising the amino acid sequence of SEQ ID NO:26, and a CDR3L region comprising the amino acid sequence of SEQ ID NO:20.

41. The method of claim 1, wherein the multispecific antibody comprises an anti-BCMA antibody or an antigen-binding fragment thereof comprising a CDR1H region comprising the amino acid sequence of SEQ ID NO: 15, a CDR2H region comprising the amino acid sequence of SEQ ID NO: 16, a CDR3H region comprising the amino acid sequence of SEQ ID NO: 17, a CDR1L region comprising the amino acid sequence of SEQ ID NO: 18, a CDR2L region comprising the amino acid sequence of SEQ ID NO: 19, and a CDR3L region comprising the amino acid sequence of SEQ ID NO:20.

42. The method of claim 1, wherein the multispecific antibody comprises an anti-BCMA antibody or an antigen-binding fragment thereof comprising a VH comprising an amino acid sequence that is at least 90% identical to, at least 95% identical to, at least 99% identical to, or identical to the amino acid sequence of SEQ ID NO: 10; and a VL comprising an amino acid sequence that is at least 90% identical to, at least 95% identical to, at least 99% identical to, or identical to the amino acid sequence of SEQ ID NO: 13.

43. The method of claim 1, wherein the multispecific antibody comprises an anti-BCMA antibody or an antigen-binding fragment thereof comprising a VH comprising an amino acid sequence that is at least 90% identical to, at least 95% identical to, at least 99% identical to, or identical to the amino acid sequence of SEQ ID NO: 9; and a VL comprising an amino acid sequence that is at least 90% identical to, at least 95% identical to, at least 99% identical to, or identical to the amino acid sequence of SEQ ID NO: 11.

44. The method of claim 1, wherein the multispecific antibody comprises five polypeptides comprising the amino acid sequences of SEQ ID NO:48, SEQ ID NO:58, SEQ ID NO: 59, SEQ ID NO:60, and SEQ ID NO:60 respectively.

45. The method of claim 1, wherein the multispecific antibody comprises five polypeptides comprising the amino acid sequences of SEQ ID NO:48, SEQ ID NO:61, SEQ ID NO:62, SEQ ID NO:63, and SEQ ID NO:63 respectively.
